(19) United States
(12) Patent Application Publication
(10) Pub. No.: US 2005/0170360 A1
(43) Pub. Date: Aug. 4, 2005

(54) Variant neuronal nicotinic alpha-7 receptor and methods of use

(76) Inventors: Roger L. Papke, Gainesville, FL (US); Andon Placzek, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/769,085

(22) Filed: Jan. 30, 2004

(51) Int. Cl. C12Q 1/68; G01N 33/53; G01N 33/567

(52) U.S. Cl. 435/6; 435/7.2

(57) ABSTRACT

The present invention relates to a variant of the nicotinic acetylcholine receptor (nAChR) α7 subunit having a substitution within its second transmembrane (TM2) domain. Specifically, the sixth amino acid position within the TM2 domain has the point mutation T→S, such that threonine-244 becomes serine-244. Advantageously, the α7 variant of the present invention retains the essential drug sensitivities of the wild-type α7 receptor, but does not exhibit the response-limiting form of fast desensitization. Therefore, the α7 variant is a “gain of function” mutant that is particularly useful for testing new pharmacological agents. The present invention includes the T6′S variant TM2 domain, T6′S variant α7 subunit, and T6′S variant nACh receptor polypeptides, polynucleotides encoding these polypeptides, recombinant hosts expressing these polynucleotides, and assays utilizing the T6′S variant TM2 domain, T6′S variant α7 subunit, and/or T6′S variant nACh receptor.

The subject invention was made with government support under a research project supported by National Institutes of Health Grant No. GM57481-01A2. The federal government may have certain rights in this invention.

### BACKGROUND OF INVENTION

Nicotinic acetylcholine receptors (nAChRs) have long been recognized as the primary postsynaptic effector in vertebrate neuromuscular transmission, as well as having a clearly established role in ganglionic neurotransmission. In recent years, nicotinic receptors have also been identified as being important functional molecules in central neurons (reviewed in (McGehee, D. S. and Role, L. W., Annu. Rev. Physiol., 57:521-546, 1995; Dani, J. A., Biol. Psychiatry, 49:166-174, 2001)). There are multiple types of nAChRs in the brain associated with synaptic function, signal processing or cell survival. Based on differences in function, pharmacology, and tissue distribution, it is possible to categorize the larger family of nAChRs broadly into three major subfamilies. These are the muscle-type receptors, the neuronal beta subunit-containing receptors that bind agonist with relatively high affinity in the desensitized state, and the homomeric alpha subunit receptors, typified by α7.

Concomitant with the evolution of these diverse forms of the nAChR and the functional radiation of these receptor subtypes into various tissues where each subtype performs highly specialized functions, a large amount of sequence divergence has emerged. There is less than 40% amino acid sequence identity between the alpha subunit of muscle type receptors (α1) and the α7-type receptor found in the brain. It has generally been assumed that the functional differences that exist among these receptor subtypes are emgergent properties of the collective sequence differences. Nonetheless, it is well appreciated that specific residues, conserved across multiple subtypes, can be key to features common to all those subtypes. Several such foci of important conserved sequence are in the pore-forming second transmembrane domain, and in this domain the sequence identity between α1 and α7 is increased to 60%.

Both muscle-type and homomeric α7 nAChRs are widely expressed in mammals and bind the snake toxin, α-Btx. Despite these similarities, there are numerous important functional and pharmacological differences between the muscle and α7 receptors. For example, α7 receptors have high divalent ion permeability, show inward rectifying current-voltage relationships, and have unique agonist concentration-dependent macroscopic response kinetics (Decker, E. R. and Dani, J. A. 1990, J. Neurosci., 10(10):3413-3420; Sands et al., 1993, Biophys. J., 65:2614-2621; Seguela et al., 1993, J. Neurosci., 13(2):596-604; Vernino et al., 1994, J. Neurosci, 14:5514-5524).

Previous work has indicated that amino acid residues in the TM2 domain of the beta subunit of heteromeric nAChRs are important regulators of nAChR pharmacology. Specifically, substitution of the TM2 6′ (according to the numbering scheme proposed by Miller et al. (Miller, C. 1989, Neuron, 2:1195-1205)) phenylalanine of the muscle β1 subunit for the neuronal β4 subunit serine residue increased sensitivity to nicotine. This study also showed that substitution of β1 subunit amino acid sequence at the TM2 6′ and 10′ positions of the neuronal β4 subunit reduced inhibition by the ganglionic blocker mecamylaminie (Webster et al., 1999, Br. J. Pharmacol, 127:1337-1348).

The therapeutic targeting of nicotinic receptors in the brain requires the identification of drugs which may be selective for their ability to activate or inhibit a limited range of these receptor subtypes. An important nAChR in the brain is the α7 subtype, which has high permeability to the calcium ion. This receptor subtype has been implicated to have an important role in improving memory-related behaviors and slowing neuronal degeneration, as may occur in aging or dementia. The activation of α7 receptors has also been suggested as a therapeutic approach for treating schizophrenia, Alzheimer's disease, inflammation, and other pathologies. (Salamone, F. et al., MJM, 2000, 5:90-97; Cooper, E. C. and Jan, L. Y. Proc. Natl. Acad. Sci. USA, 1999, 96:4759-4766; Sharples, C. and Wonnacott, S. Neuronal Nicotinic Receptors, October 2001, 19:1-12; Mihailescu, S. and Drucker-Colin, R. Arch. Med. Res., 2000, 31:131-144; Papke, R. L. et al., Euro. J. Pharm., 2000, 393:179-195; Newhouse, P. A. and Kelton, M. Pharm. Acta Helv., 2000, 74:91-101; Newhouse, P. A. et al., Clin. Pharm., 1997, 11:206-228; Lloyd, G. K. and Williams, M. J. Pharm. Exp. Therapies, 2000, 292:461-467; Hollady, M. W. et al., J. Med. Chem., 1997, 40:4169-4194; Benowitz, N. L. Annu. Rev. Pharm. Toxicol., 1996, 36:597-613; Freedman, R. et al., Harvard Rev. Psych., 1994, 2:179-192; and Freedman, R. et al., J. Chem. Neuroanatomy, 2000, 20:299-306).

The development of drugs for targeting the α7 receptor therapeutically has been impeded by the fact that the wild-type receptor exhibits a unique concentration-dependent fast desensitization that makes it difficult to study with large-scale drug screens. It would be advantageous to have available a variant of the wild-type α7 nAChR that does not exhibit the response-limiting form of fast desensitization. Variant α7 nAChRs have been reported previously (Galzi J L et al., Nature, 359:500-505, 1992; Bertrand D. et al., Proc. Natl. Acad. Sci. U.S.A., 90(15):6971-6975, 1993; Revah F. et al., Nature 353:846-849, 1991; Hogg R. C. et al., J. Biol. Chem., 278(29):26908-26912, July 2003, Epub May 13, 2003; Tonini R. et al., Neuropharmacology, 44(6):765-771, May 2003; Eddins D. et al., Am. J. Cell Physiol., 283(5):C1454-C1460, 2003; Lyford L. K. et al., Biochem Biophys. Acta. 10; 1559(1): 69-78, 2002; Orr-Urtreger A et al., J. Neurochem., 74(5):2154-2166, 2000; Palma E, et al., Proc. Natl. Acad. Sci. U.S.A., 96(23):13421-13426, 1999; Maggi L. et al., Mol. Psychiatry, 3(4):350-355, 1998; Bertrand S. et al., Neuroreport, 8(16):3591-3596, 1997; Palma E. et al., Proc. Natl. Acad. Sci. U.S.A., 94(4):1539-1543, 1997; Palma E. et al., Proc. Natl. Acad. Sci. USA., 93(20):11231-11235, 1996; Bertrand D. et al., Proc. Natl. Acad. Sci. U.S.A., 89(4):1261-1265, 1992; U.S. Pat. No. 6,323,000 (Briggs et al.), filed Dec. 20, 1996; and U.S. Pat. No. 6,683,157 (Briggs et al.), filed Sep. 18, 2001.

“Gain of function” mutants of the α7 receptor have previously been described; however, these mutants respond very differently to experimental drugs from wild-type receptors. The ideal “gain of function” mutant would retain the essential drug sensitivities of the wild-type native α7 receptors and, therefore, provide a much sought after model for testing new drugs.

### BRIEF SUMMARY OF THE INVENTION

The present invention relates to a variant of the nicotinic acetylcholine receptor (nAChR) α7 subunit having a substitution at the 6′ position of the second transmembrane domain (referred to as M2 or TM2). Specifically, a threonine has been substituted with serine. For example, in the case of the rat (Rattus norvegicus) α7 subunit, threonine-244 has been substituted with serine. In accordance with the numbering scheme proposed by Miller (Neuron, 1989, 2:1195-1205), the variant is referred to herein as the T6′S mutant or T6′S variant (because the point mutation T→S is in the sixth amino acid position of the TM2 domain).

Thus, the nucleotide sequence encoding the T6′S variant differs from the coding sequence of the wild-type rat α7 subunit in containing a codon (sequence of three nucleotides) encoding serine, instead of threonine, at nucleotides 799 to 781 of the coding sequence (nucleotides 733-735 excluding signal sequence).

The T6′S variant exhibits pharmacological and electrophysiological characteristics that are very similar to the wild-type α7 subunit. However, unexpectedly, the T6′S variant exhibits much slower desensitization in comparison to the wild-type α7 subunit and is, therefore, a “gain of function” mutant. In fact, the T6′S variant of the present invention substantially eliminates the fast desensitization rate observed in the wild-type α7 subunit. This distinguishing feature, combined with the variant's similarities to the wild-type α7 subunit, makes the T6′S variant particularly useful for drug screening purposes.

Accordingly, one aspect of the present invention is an isolated polynucleotide that encodes a variant of the nAChR TM2 domain of the α7 subunit, wherein the variant of the nAChR TM2 domain comprises the amino acid sequence of SEQ ID NO:2, or a functional fragment or homologue thereof, wherein the functional fragment or homologue contains the T→S substitution at the T6′S position. In one embodiment, the polynucleotide comprises the nucleic acid sequence of SEQ ID NO:18. In other embodiments, the polynucleotide comprises the nucleic acid sequence of SEQ ID NO:21, SEQ ID NO:22, SEQ ID NO:23, SEQ ID NO:24, or SEQ ID NO:25 which are degenerate variants of SEQ ID NO:18.

The present invention further includes an isolated polynucleotide that encodes a variant of the wild-type nAChR α7 subunit. In one embodiment, the polynucleotide comprises the nucleic acid sequence of SEQ ID NO:4 or nucleotides 67 to 1509 of SEQ ID NO:4. In other embodiments, the polynucleotide is a degenerate variant of SEQ ID NO:4. For example, the degenerate variant can comprise the nucleic acid sequence of SEQ ID NO:26 or nucleotides 67 to 1509 of SEQ ID NO:26; or SEQ ID NO:27 or nucleotides 67 to 1509 of SEQ ID NO:27; or SEQ ID NO:28 or nucleotide 67 to 1509 of SEQ ID NO:28; or SEQ ID NO:29 or nucleotides 67 to 1509 of SEQ ID NO:29; or SEQ ID NO:30 or nucleotides 67 to 1509 of SEQ ID NO:30.

In another aspect, the present invention includes an isolated polypeptide that is a variant of the wild-type TM2 domain of the α7 subunit (SEQ ID NO:1), wherein the variant domain comprises the amino acid sequence of SEQ ID NO:2.

The present invention further includes isolated polypeptides that are variants of the wild-type nAChR α7 subunits of rat (amino acids 23 to 502 of SEQ ID NO:5), human (amino acids 23 to 502 of SEQ ID NO: 7), mouse (amino acids 23 to 502 of SEQ ID NO: 9) monkey (amino acids 23 to 502 of SEQ ID NO: 11), cow (amino acids 23 to 502 of SEQ ID NO: 13), chicken (amino acids 23 to 502 of SEQ ID NO: 13), zebra fish (amino acids 23 to 502 of SEQ ID NO: 15) wherein the variants of the wild-type nAChR α7 subunit comprise amino acids 23 to 502 of SEQ ID NO:20, or a functional fragment or homologue thereof, wherein the functional fragment or homologue contains a TM2 domain with the T→S substitution at the T6′S position (e.g., functional fragments or homologues comprising the amino acid sequence of SEQ ID NO:2).

In other aspects, the present invention concerns an isolated nAChR (also referred to herein as the T6′S variant nAChR) comprising the T6′S variant TM2 domain (SEQ ID NO:2) or a T6′S variant α7 subunit (such as amino acids 23 to 502 of SEQ ID NO:20), and an isolated polynucleotide encoding the T6′S variant nAChR or α7 subunit. The isolated nAChR may comprise other wild-type subunits and/or variant subunits, in addition to the T6′S variant α7 subunit of the present invention.

In another aspect, the present invention concerns a recombinant vector comprising a polynucleotide of the invention. In another aspect, the present invention concerns genetically modified host cells that have been transformed or transfected with vectors comprising a polynucleotide of the invention. In another aspect, the present invention concerns methods of producing recombinant polypeptides for the treatment of neurological conditions (e.g., neurodegenerative processes), enzymatic function, affective disorders, and immunofunction, using such cells. In another aspect, the present invention concerns methods of treating pathologic conditions, such as neurological disorders, by administering a polynucleotide encoding a T6′S variant of the present invention.

In another aspect, the present invention concerns agents (e.g., antisense polynucleotides, compounds), such as antagonists, which are useful in treating conditions such as neurological conditions (e.g., neurodegenerative processes), enzymatic function, affective disorders, and immunofunction. Methods of treating patients using these antisense polynucleotides and compounds are also provided.

In another aspect, the present invention pertains to methods and reagents for detecting the T6′S variant.

In another aspect, the present invention is directed to a method of expressing a polynucleotide of the subject invention in a cell. In one embodiment, a polynucleotide encoding the T6′S variant is expressed in the cell. In another embodiment, a polynucleotide encoding an nAChR comprising the T6′S variant subunit is expressed in the cell.

In another aspect, the present invention is directed to a method of identifying agents that modulate the activity of the T6′S variant and receptors containing the variant subunit, and to a method of identifying cytoprotective or other therapeutic agents, using recombinant cells of the present invention.

### BRIEF DESCRIPTION OF DRAWINGS

FIG. 1 shows a rendering of the nicotinic acetylcholine receptor (nAChR) accompanied by a hydrophobicity plot of the α7 nAChR subunit.

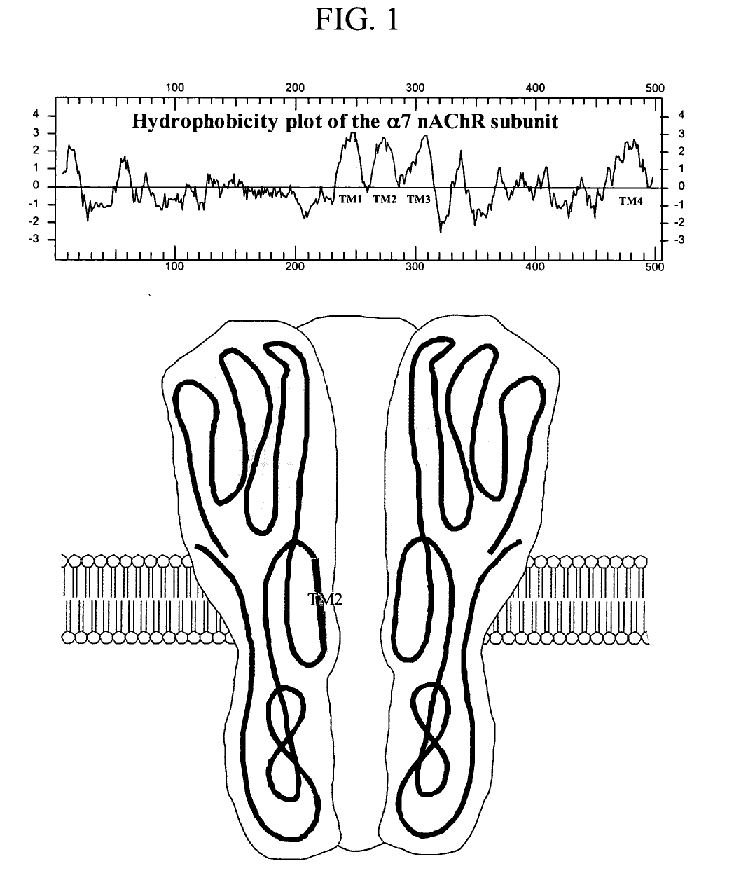

FIG. 2 shows the amino acid sequences of the wild-type α7 TM2 domain, wild-type β2 TM2 domain, wild-type β4 TM2 domain, wild-type β1 TM2 domain, and two α7 TM2 mutants having amino acid substitutions at the sixth and tenth amino acid positions of the TM2 domain. The numbering of specific residues of the second membrane spanning region is in accordance with that proposed by Miller et al. (Neuron, 2:1195-1205, 1989).

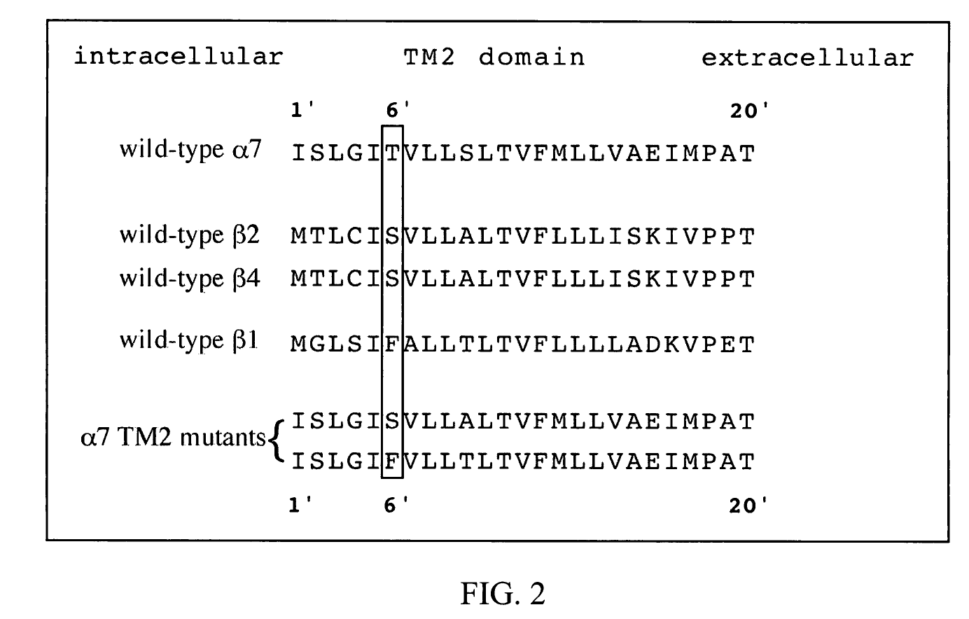

FIG. 3 shows acetylcholine (ACh) evoked responses from oocytes expressing wild-type α7 (300 μM ACh), rat α7 TM2 T6′F (3 μM ACh), rat α7 TM2 T6′S (100 μM ACh). The inset represents the same current traces normalized to one another for the purpose of comparison.

FIGS. 4A-4C show ACh evoked normalized responses from oocytes expressing wild-type α7, rat α7 TM2 T6′S, and rat α7 TM2 T6° F., respectively, at the ACh concentrations indicated.

FIGS. 5A-5C show electrophysiological chacterization of wild-type and mutant α7. FIGS. 5A and 5B demonstrate that the T6° F. mutant receptor shows a nearly total loss of permeability to barium. FIG. 5C demonstrates that the T6′S mutant exhibits a confounding biphasic current that occurs near the chloride reversal potential.

FIGS. 6A-6D show electrophysiological traces demonstrating that substitution of the muscle β1 subunit phenylalanine at the TM2 6′ position confers the predicted sensitivity to succinylcholine, whereas the T6′S mutant is similar to wild-type α7.

FIG. 7 shows the concentration-response relationships for wild-type α7 and TM2 6′ mutants showing selectivity of succinylcholine (SuCh) for the T6° F. mutant.

FIGS. 8A and 8B show ACh evoked responses from GH4Cl cells expressing the T6′S mutant and the wild-type α7 receptor, respectively.

### BRIEF DESCRIPTION OF SEQUENCES

SEQ ID NO:1 is the amino acid sequence of the TM2 domain of the wild-type α7 subunit (the threonine in the sixth amino acid position of TM2 is underlined and bold for reference), which has 100% homology among Homo sapiens (human), Mucaca mulatto (rhesus monkey), Rattus norvegicus (rat), Mus musculus (mouse), Bos taurus (domestic cow), Gallus gallus (chicken), Danio rerio (zebrafish):

ISLGITVLLSLTVFMLLVAE

SEQ ID NO:2 is the amino acid sequence of the T6′S variant of the second transmembrane (TM2) domain (the serine in the sixth amino acid position of TM2 is underlined and bold for reference):

ISLGISVLLSLTVFMLLVAE

SEQ ID NO:3 is the nucleotide coding sequence of the TM2 domain of the wild-type rat α7 subunit (the codon encoding the threonine at the sixth amino acid of TM2 is underlined and bold for reference):

atctctcttggaataactgtcttactttct

ctgactgtcttcatgctgcttgtggctgag

SEQ ID NO:4 is the nucleotide coding sequence of the wild-type rat α7 subunit, including nucleotides 1-66 (underlined), which encode a signal peptide that is not present in the mature α7 receptor subunit (the TM2 domain at nucleotides 784-843 is underlined and the codon for the sixth amino acid (6′) of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaataactgtc
ttactttctctgactgtcttcatgctgcttgtggctgagatcatgccagcaacatctgattctgtgcccttgatagcacaatacttcgccagc
accatgatcatcgtgggcctctctgtagtggtgacagtgattgtgctgagatatcaccaccatgaccctgatggtggcaaaatgcctaag
tggaccagaatcattctcctgaactggtgtgcatggtttctgcgcatgaagaggcccggagaggacaaggtgcggccagcttgtcag
cacaagcctcggcgctgcagcctggccagtgtggagctgagtgcaggtgctgggccacccaccagcaatggcaacctgctctacat
tggcttccgaggcctggagggcatgcactgtgccccaactccagactctggggtcgtatgtggccgtttggcctgctccccaacacat
gatgagcacctcatgcacggtgcacacccctctgatggggaccccgacctggccaagatcctggaggaggtccgctacatcgccaa
ccgcttccgctgccaggacgagagtgaggtgatctgcagtgaatggaagtttgcagcctgcgtggtggaccgcttgtgcctcatggcc
ttttcggtctttaccatcatctgtaccatcggcatcctcatgtcagctccaaactttgtggaggctgtgtccaaagactttgcttaa

(NCBI Accession No. NM_{—}012832; Khirog S. et al., J. Physiol. (Lond.) 540(Pt. 2):425-434, 2002; Oshikawa J. et al., Am. J. Physiol., Cell Physiol., 285(3):C567-C574, 2003; which are incorporated herein by reference in their entireties).

SEQ ID NO:5 is the amino acid sequence of the wild-type rat α7 subunit, including amino acids 1-22 (underlined), which represent a signal peptide that is not present in the mature receptor subunit (the TM2 domain at amino acids 262-281 is underlined and the 6′ threonine of TM2 is bold for reference):

MCGGRGGIWLALAAALLHVSLQGEFQRRLYKELVKNYNPLERPVANDSQPLTVYFS
LSLLQIMDVDEKNQVLTTNIWLQMSWTDHYLQWNMSEYPGVKNVRFPDGQIWKPD
ILLYNSADERFDATFHTNVLVNASGHCQYLPPGIFKSSCYIDVRWFPFDVQQCKLKF
GSWSYGGWSLDLQMQEADISSYIPNGEWDLMGIPGKRNEKFYECCKEPYPDVTYTV
TMRRRTLYYGLNLLIPCVLISALALLVFLLPADSGEKISLGITVLLSLTVFMLLVAEIM
PATSDSVPLIAQYFASTMIIVGLSVVVTVIVLRYHHHDPDGGKMPKWTRIILLNWCA
WFLRMKRPGEDKVRPACQHKPRRCSLASVELSAGAGPPTSNGNLLYIGFRGLEGMH
CAPTPDSGVVCGRLACSPTHDEHLMHGAHPSDGDPDLAKILEEVRYIANRFRCQDES
EVICSEWKFAACVVDRLCLMAFSVFTIICTIGILMSAPNFVEAVSKDFA

The α7 subunit protein has approximately 90% homology among rat, chick, and humans.

SEQ ID NO:6 is the nucleotide coding sequence of the wild-type human α7 subunit, including nucleotides 1-66 (underlined), which encode a signal peptide that is not present in the mature α7 receptor subunit (the TM2 domain at nucleotides 784-843 is underlined and the codon for the 6′ amino acid of TM2 (threonine) is bold for reference):

atgcgctgctcgccgggaggcgtctggctggcgctggccgcgtcgctcctgcacgtgtccctgcaaggcgagttccagaggaagct
ttacaaggagctggtcaagaactacaatcccttggagaggcccgtggccaatgactcgcaaccactcaccgtctacttctccctgagc
ctcctgcagatcatggacgtggatgagaagaaccaagttttaaccaccaacatttggctgcaaatgtcttggacagatcactatttacagt
ggaatgtgtcagaatatccaggggtgaagactgttcgtttcccagatggccagatttggaaaccagacattcttctctataacagtgctg
atgagcgctttgacgccacattccacactaacgtgttggtgaattcttctgggcattgccagtacctgcctccaggcatattcaagagttc
ctgctacatcgatgtacgctggtttccctttgatgtgcagcactgcaaactgaagtttgggtcctggtcttacggaggctggtccttggatc
tgcagatgcaggaggcagatatcagtggctatatccccaatggagaatgggacctagtgggaatccccggcaagaggagtgaaag
gttctatgagtgctgcaaagagccctaccccgatgtcaccttcacagtgaccatgcgccgcaggacgctctactatggcctcaacctgc
tgatcccctgtgtgctcatctccgccctcgccctgctggtgttcctgcttcctgcagattccggggagaagatttccctggggataacag
tcttactctctcttaccgtcttcatgctgctcgtggctgagatcatgcccgcaacatccgattcggtaccattgatagcccagtacttcgcc
agcaccatgatcatcgtgggcctctcggtggtggtgacggtgatcgtgctgcagtaccaccaccacgaccccgacgggggcaagat
gcccaagtggaccagagtcatccttctgaactggtgcgcgtggttcctgcgaatgaagaggcccggggaggacaaggtgcgcccg
gcctgccagcacaagcagcggcgctgcagcctggccagtgtggagatgagcgccgtggcgccgccgcccgccagcaacgggaa
cctgctgtacatcggcttccgcggcctggacggcgtgcactgtgtcccgacccccgactctggggtagtgtgtggccgcatggcctg
ctcccccacgcacgatgagcacctcctgcacggcgggcaaccccccgagggggacccggacttggccaagatcctggaggaggt
ccgctacattgccaaccgcttccgctgccaggacgaaagcgaggcggtctgcagcgagtggaagttcgccgcctgtgtggtggacc
gcctgtgcctcatggccttctcggtcttcaccatcatctgcaccatcggcatcctgatgtcggctcccaacttcgtggaggccgtgtcca
aagactttgcgtaa

(NCBI Accession No. NM000746; Chini B. et al., Genomics 19(2):379-381, 1994; Burhaus L. et al., Parkinsonism Relat. Disord., 9(5):243-246, 2003; which are incorporated by reference herein in their entireties).

SEQ ID NO:7 is the amino acid sequence of the wild-type human α7 subunit, including amino acids 1-22 (underlined), which represent a signal peptide that is not present in the mature receptor subunit (the TM2 domain at amino acids 262-281 is underlined and the codon for the 6′ amino acid of TM2 (threonine) is bold for reference for reference):

MRCSPGGVWLALAASLLHVSLQGEFQRKLYKELVKNYNPLERPVANDSQPLTVYFS
LSLLQIMDVDEKNQVLTTNIWLQMSWTDHYLQWNVSEYPGVKTVRFPDGQIWKPD
ILLYNSADERFDATFHTNVLVNSSGHCQYLPPGIFKSSCYIDVRWFPFDVQHCKLKFG
SWSYGGWSLDLQMQEADISGYIPNGEWDLVGIPGKRSERFYECCKEPYPDVTFTVT
MRRRTLYYGLNLLIPCVLISALALLVFLLPADSGEKISLGITVLLSLTVFMLLVAEIMP
ATSDSVPLIAQYFASTMIIVGLSVVVTVIVLQYHHHDPDGGKMPKWTRVILLNWCA
WFLRMKRPGEDKVRPACQHKQRRCSLASVEMSAVAPPPASNGNLLYIGFRGLDGV
HCVPTPDSGVVCGRMACSPTHDEHLLHGGQPPEGDPDLAKILEEVRYIANRFRCQDE
SEAVCSEWKFAACVVDRLCLMAFSVFTIICTIGILMSAPNFVEAVSKDFA

SEQ ID NO:8 is the nucleotide coding sequence of the wild-type mouse α7 subunit, including nucleotides 1-66 (underlined), which encode a signal peptide that is not present in the mature α7 receptor subunit (the TM2 domain at nucleotides 784-843 is underlined and the codon for the 6′ amino acid of TM2 (threonine) is bold for reference):

atgtgcggccggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gcctcctgcagatcatggatgtggatgagaagaaccaagttttaaccaccaacatttggcttcaaatgtcttggacagatcactatttgca
gtggaacatgtctgagtaccccggagtgaaaaatgttcgttttccagatggccagatttggaaaccagacattctcctctataacagtgc
agatgaacgctttgatgccacattccacaccaacgtcttggtgaatgcatctgggcattgccagtatctccctccaggcatattcaagag
ctcctgctacatcgatgtacgctggttcccttttgatgtgcagcagtgcaaactgaagtttgggtcctggtcctatggagggtggtccttg
gacctgcagatgcaagaggcagatatcagcagctatatccccaatggagaatgggatctcatgggaatccctggcaaaaggaatga
gaagttctatgaatgctgcaaagagccatacccagatgtcacctacacagtaaccatgcgccgtaggacactctactatggcctcaacc
tgctcattccttgtgtgctcatttcagccctggctttgctggtattcttgctgcctgcagactctggagagaaaatctctcttggaataactgt
cttactttctctgactgtcttcatgctgcttgtggctgagatcatgccagcaacatctgattccgtgcccttgatagcacagtacttcgccag
caccatgatcatcgtgggcctctcagtggtcgtgacagtgattgtgctgcgatatcaccaccatgaccctgatggtggcaaaatgccta
agtggaccaggatcattcttctgaattggtgtgcatggtttctgcgcatgaagaggccgggagaggacaaggtgcggccggcttgtca
gcacaagcctcggcgctgcagcctggccagtgtggagctgagcgcaggtgcggggccaccctccagcaatggcaacctgctctac
attggcttccgaggcctggagggcatgcactgtgccccaactccagactctggggtcgtgtgtggtcgtttggcctgctccccaacac
atgatgagcacctcatgcatggtacacacccctctgatggggaccccgacctggccaagatcctggaggaggtccgctacatcgcca
accgcttccgctgccaggatgagagtgaggtgatctgcagtgagtggaagtttgcggcctgtgtggtggaccgcttgtgcctcatggc
cttttcggtttttaccatcatctgcaccatcggcatcctcatgtcagccccaaactttgtggaggctgtgtccaaagactttgcttaa

(NCBI Accession No. NM_{—}007390; Orr-Urtreger A. et al., Genomics 26(2):399-402, 1995; Saragoza P. A. et al., Brain Res. Mol. Brain Res. 117(1):15-26, 2003; which are incorporated by reference herein in their entireties).

SEQ ID NO:9 is the amino acid sequence of the wild-type mouse α7 subunit, including amino acids 1-22 (underlined), which represent a signal peptide that is not present in the mature receptor subunit (the TM2 domain at amino acids 262-281 is also underlined for reference):

MCGRRGGIWLALAAALLHVSLQGEFQRRLYKELVKNYNPLERPVANDSQPLTVYFS
LSLLQIMDVDEKNQVLTTNIWLQMSWTDHYLQWNMSEYPGVKNVRFPDGQIWKPD
ILLYNSADERFDATFHTNVLVNASGHCQYLPPGIFKSSCYIDVRWFPFDVQQCKLKF
GSWSYGGWSLDLQMQEADISSYIPNGEWDLMGIPGKRNEKFYECCKEPYPDVTYTV
TMRRRTLYYGLNLLIPCVLISALALLVFLLPADSGEKISLGITVLLSLTVFMLLVAEIM
PATSDSVPLIAQYFASTMIIVGLSVVVTVIVLRYHHHDPDGGKMPKWTRIILLNWCA
WFLRMKRPGEDKVRPACQHKPRRCSLASVELSAGAGPPSSNGNLLYIGFRGLEGMH
CAPTPDSGVVCGRLACSPTHDEHLMHGTHPSDGDPDLAKILEEVRYIANRFRCQDES
EVICSEWKFAACVVDRLCLMAFSVFTIICTIGILMSAPNFVEAVSKDFA

SEQ ID NO:10 is the nucleotide coding sequence of the wild-type rhesus monkey α7 subunit, including nucleotides 1-66 (underlined), which encode a signal peptide that is not present in the mature α7 receptor subunit (the TM2 domain at nucleotides 784-843 is underlined and the codon for the 6′ amino acid of TM2 (threonine) is bold for reference):

atgcgctgctcgcagggaggcgtctggctggctctggccgcgtcgctcctgcatgtgtccctgcaaggcgagttccagaggaagcttt
acaaggagctggtcaagaactacaaccccttggagaggcccgtggccaatgactcgcaaccgctcaccgtctacttctccctgagcc
tcctgcagatcatggacgtggatgagaagaaccaagttttaaccaccaacatttggctgcaaatgtcttggacagatcactatttacagt
ggaatgtgtcagaatatccaggggtgaagactgttcgtttcccagatggccagatttggaaaccagacattcttctctataacagtgcgg
atgagcgctttgacgccacattccacaccaacgtgttggtgaattcttctgggcattgccagtacctgcctccaggcatattcaagagttc
ctgctacatcgatgtgcgctggtttccctttgatgtgcagcattgcaaactgaagtttggatcctggtcttatggaggctggtccttggatct
gcagatgcaggaggcagatatcagtggctatatccccagtggagaatgggacctagtgggaattcccggcaagaggagtgaaaagt
tctatgagtgctgcaaagagccctaccccgatgtcaccttcacagtgaccatgcgccgcaggaccctctactacggcctcaacctgct
gatcccctgtgtgctcatctctgcccttgccctgctggtgttcctgcttcctgcagattccggggagaagatttccctggggataacagtc
ttactctctctcactgtcttcatgctgctcgtggctgagatcatgcccgcaacatctgattcagtaccattgatagcccagtacttcgccag
caccatgatcatcgtgggcctctccgtggtggtgacggtgatcgtgctgcagtaccaccaccacgaccccgacgggggcaagatgc
ccaagtggaccagagtcatccttctgaactggtgcgcgtggttcctgcgcatgaagaggccgggagaggataaggtgcgcccggcc
tgccagcacaagcagcgccgctgcagcctggccagcgtggagatgagcgccgtggcgccgccgcccgccagcaacgggaacct
gctgtacatcggcttccgcggcctggacggcatgcattgcgccccgacccccgactccggggtggtgtgcggccgcatggcctgct
cccccacgcacgacgagcacctcctgcacggtgggcagccccccgagggggacccggacctggccaagatcctggaggaggtc
cgctacatcgccaaccgctttcgctgccaggacgaaagcgaggcggtctgcagtgagtggaagttcgccgcctgcgtggtggaccg
cctgtgcctcatggccttctcggtcttcaccatcatctgcaccatcggcatcctgatgtcggctcccaacttcgtggaggccgtgtccaa
agactttgcgtaa

(NCBI Accession No. AF486623).

SEQ ID NO:11 is the amino acid sequence of the wild-type rhesus monkey α7 subunit, including amino acids 1-22 (underlined), which represent a signal peptide that is not present in the mature receptor subunit (the TM2 domain at amino acids 262-281 is also underlined for reference):

MRCSQGGVWLALAASLLHVSLQGEFQRKLYKELVKNYNPLERPVANDSQPLTVYF
SLSLLQIMDVDEKNQVLTTNIWLQMSWTDHYLQWNVSEYPGVKTVRFPDGQIWKP
DILLYNSADERFDATFHTNVLVNSSGHCQYLPPGIFKSSCYIDVRWFPFDVQHCKLKF
GSWSYGGWSLDLQMQEADISGYIPSGEWDLVGIPGKRSEKFYECCKEPYPDVTFTVT
MRRRTLYYGLNLLIPCVLISALALLVFLLPADSGEKISLGITVLLSLTVFMLLVAEIMP
ATSDSVPLIAQYFASTMIIVGLSVVVTVIVLQYHHHDPDGGKMPKWTRVILLNWCA
WFLRMKRPGEDKVRPACQHKQRRCSLASVEMSAVAPPPASNGNLLYIGFRGLDGM
HCAPTPDSGVVCGRMACSPTHDEHLLHGGQPPEGDPDLAKILEEVRYIANRFRCQDE
SEAVCSEWKFAACVVDRLCLMAFSVFTIICTIGILMSAPNFVEAVSKDFA

SEQ ID NO:12 is the nucleotide coding sequence of the wild-type domestic cow α7 subunit, -including nucleotides 1-57 (underlined), which encode a signal peptide that is not present in the mature α7 receptor subunit (the TM2 domain at nucleotides 775-834 is underlined and the codon for the 6′ amino acid of TM2 (threonine) is bold for reference):

atgcgcggctcgttgtgcctggcgctggcagcgtcgatcttgcacgtgtccctgcaaggcgagttccagaggaagctctacaaagac
ctggtgaagaactacaaccccttggagaggccggtggccaacgactcgctgccgctcacagtctacttctccctgagcctcctgcaga
tcatggacgtggatgagaagaaccaggtgttgaccaccaacatttggctacaaatgacttggacagatcactatttacagtggaatgca
tccgagtatccaggcgtgaagacggttcgtttcccagacggccagatctggaagccagacattctcctctacaacagtgctgatgaac
ggtttgatgctacgttccacaccaatgtgttagtgaattcttctgggcactgccagtacctccctccaggcatctttaagagctcctgctac
atcgatgtgcgctggttcccatttgatgtgcagcagtgcaaactgaagtttgggtcctggtcttatggaggatggtccttggatctgcaaa
tgcaggaggcagacatcagcggctatatcccaaatggagaatgggaccttgtgggggtcttgggcaagcggagtgagaagttctac
gagtgctgcaaagaaccctacccagacgtgaccttcacggtcagcatccgccgcaggactctctactacggcctcaacctgctcatcc
cctgtgtgctcatctctgccctagccctcctcgtcttcttgctccctgcagactctggagagaaaatctccctgggaatcacagttttactc
tccctcaccgtcttcatgctgctggtagctgagatcatgcctgcgacctctgactcagtgcccttaatagcccagtacttcgccagcacc
atgatcatcgtgggcctctctgtggttgtcacggtgatcgtgctacagtaccaccaccacgaccctgatggtggcaagatgcccaagtg
gaccagagtcgtcctcctgaactggtgtgcctggttccttcgcatgaagaggccgggggaggacaaggtgcggccagcctgccagc
acaacgagcggcgctgcagcctggccagcgtggagatgagcgcagtggcggggccgccggccaccaatggcaacctgctgtac
atcggctttcgcggcctggacaccatgcactgcgcacccacccctgactcaggggtcgtgtgcggccgcgtggcctgctccccaac
ccatgacgagcacctgctgcacgccggccagccctctgagggggacccggacctagccaagatcctggaggaggtgcgctacatt
gcccaccgcttccgctgccaggatgagagtgaggccgtgtgcagtgagtggaagttcgcggcctgtgtggtggaccgcctgtgtctc
atggccttctccgtcttcaccatcctctgcaccattggcatcctgatgtcggcgcccaacttcgttgaagctgtgtccaaagacttcgcttg
a

(NCBI Accession No. NM_{—}174515; Garcia-Guzman, M. et al., Eur. J. Neurosci. 7(4):647-655, 1995; Maneu V. et al., Ann. N.Y. Acad. Sci. 971:165-167, 2002; which are incorporated by reference herein in their entirety).

SEQ ID NO:13 is the amino acid sequence of the wild-type cow α7 subunit, including amino acids 1-19 (underlined), which represent a signal peptide that is not present in the mature receptor subunit (the TM2 domain at amino acids 259-278 is also underlined for reference):

MRGSLCLALAASILHVSLQGEFQRKLYKDLVKNYNPLERPVANDSLPLTVYFSLSLL
QIMDVDEKNQVLTTNIWLQMTWTDHYLQWNASEYPGVKTVRFPDGQIWKPDILLY
NSADERFDATFHTNVLVNSSGHCQYLPPGIFKSSCYIDVRWFPFDVQQCKLKFGSWS
YGGWSLDLQMQEADISGYIPNGEWDLVGVLGKRSEKFYECCKEPYPDVTFTVSIRR
RTLYYGLNLLIPCVLISALALLVFLLPADSGEKISLGITVLLSLTVFMLLVAEIMPATS
DSVPLIAQYFASTMIIVGLSVVVTVIVLQYHHHDPDGGKMPKWTRVVLLNWCAWFL
RMKRPGEDKVRPACQHNERRCSLASVEMSAVAGPPATNGNLLYIGFRGLDTMHCA
PTPDSGVVCGRVACSPTHDEHLLHAGQPSEGDPDLAKILEEVRYIAHRFRCQDESEA
VCSEWKFAACVVDRLCLMAFSVFTILCTIGILMSAPNFVEAVSKDFA

SEQ ID NO:14 is the nucleotide coding sequence of the wild-type chicken α7 subunit, including nucleotides 1-66 (underlined), which encode a signal peptide that is not present in the mature α7 receptor subunit (the TM2 domain at nucleotides 784-843 is underlined and the codon for the 6′ amino acid of TM2 (threonine) is bold for reference):

atgggcctccgggcgctgatgctgtggctgctggcggcggcggggctcgtgcgcgagtccctgcaaggagagttccaaaggaagc
tgtacaaggagctgctgaagaactacaaccctctggaacgaccagttgcaaatgactcccagccgctcactgtctatttcactctcagc
ctcatgcagatcatggatgtggatgaaaagaatcaagtattaacaacaaacatctggctacaaatgtactggacagatcattacttacag
tggaatgtgtctgaataccctggagtgaagaacgtccgttttcctgatggactgatttggaagccagatattcttctctataacagtgctga
tgaaagatttgatgctacatttcacactaatgttttagtcaattcttcgggacactgccaatatctgccaccaggcatatttaaaagctcatg
ctacatagacgtgcgttggtttccatttgatgttcagaagtgcaatctgaagtttggatcttggacatatggaggctggtccttagacttaca
aatgcaagaagcagatatatccggctatatttcaaatggagagtgggatttagtaggaattcctgggaagagaactgagagcttttatga
gtgctgtaaagaaccatacccagatatcacattcacagtaaccatgagacgcagaactctctactacgggctcaaccttcttattccctgt
gtactgatatcagcacttgccttattagtctttctgcttccagcagactcaggagaaaagatctcactaggtataacagttttattgtctctca
ccgtcttcatgttactcgtggctgaaattatgccagcaacatctgattctgtgcccttaattgctcagtattttgccagcaccatgattattgtt
ggcctctctgttgttgtcactgttatcgttctacaataccatcatcacgatccagatgggggaaaaatgcctaaatggacaagagtcatcc
ttctgaattggtgtgcttggtttctgaggatgaagagaccaggggaagataaagtgcgtcccgcctgtcaacataaacagcgccgatg
cagcctgtcaagcatggagatgaacactgtgagtggtcagcaatgcagtaatgggaacatgctgtatattgggtttcgagggctggat
ggggttcactgcacacccaccactgattcaggggtgatctgtgggaggatgacctgttcaccaacagaggaagaaaatcttctgcaca
gtggccacccctctgaaggcgacccagatttggctaagatcctggaagaggtcagatacattgcaaacaggttcagagaccaggatg
aagaagaagccatttgcaacgaatggaagtttgcagcctctgtagtagatcggctctgcttgatggcattttcggtcttcaccatcatttgt
acaattggcatcttaatgtcagcaccaaactttgtagaggctgtctctaaagattttgcttaa

(NCBI Accession No. X52295; Schoepfer R. et al., Neuron 5(1):35-48, 1990; which is incorporated herein by reference in its entirety).

SEQ ID NO:15 is the amino acid sequence of the wild-type chicken α7 subunit, including amino acids 1-22 (underlined), which represent a signal peptide that is not present in the mature receptor subunit (the TM2 domain at amino acids 262-281 is also underlined for reference):

MGLRALMLWLLAAAGLVRESLQGEFQRKLYKELLKNYNPLERPVANDSQPLTVYF
TLSLMQIMDVDEKNQVLTTNIWLQMYWTDHYLQWNVSEYPGVKNVRFPDGLIWK
PDILLYNSADERFDATFHTNVLVNSSGHCQYLPPGIFKSSCYIDVRWFPFDVQKCNLK
FGSWTYGGWSLDLQMQEADISGYISNGEWDLVGIPGKRTESFYECCKEPYPDITFTV
TMRRRTLYYGLNLLIPCVLISALALLVFLLPADSGEKISLGITVLLSLTVFMLLVAEIM
PATSDSVPLIAQYFASTMIIVGLSVVVTVIVLQYHHHDPDGGKMPKWTRVILLNWCA
WFLRMKRPGEDKVRPACQHKQRRCSLSSMEMNTVSGQQCSNGNMLYIGFRGLDGV
HCTPTTDSGVICGRMTCSPTEEENLLHSGHPSEGDPDLAKILEEVRYIANRFRDQDEE
EAICNEWKFAASVVDRLCLMAFSVFTIICTIGILMSAPNFVEAVSKDFA

SEQ ID NO:16 is the nucleotide coding sequence of the wild-type zebrafish α7 subunit, including nucleotides 1-66 (underlined), which encode a signal peptide that is not present in the mature α7 receptor subunit (the TM2 domain at nucleotides 784-843 is underlined and the codon for the 6′ amino acid of TM2 (threonine) is bold for reference):

atgggaatttgggaatatactctttatttcaccaccacatgctgcctatggagtgtgtcacttcagggggagcatcagaggagactctata
gagacctgatgaaagattataacccactggagagacctgtgttcaatgacacccactcactcacggtgtatttcagcatgagcctcatg
cagattatggatgtggatgaaaagaatcaagttcttacgaccaacatttggctacagctgtactggtatgactactatcttcaatggaacg
cctcagaatatcctggagtaaccaatgtaaggtttcctgacagtcagatctggaagcctgacatcttactttataacagtgctgatgaaag
gtttgatgccacatttcatactaatgtgctggtgaactcttctggtgcctgccaatacttaccgccagggatcttcaaaagcacctgctaca
tcgacgttcgctggttcccctttgatcttcagaggtgtgatctgaaatttggctcctggacgtatggaggatggtctctggacctgcagat
gattgatgccgacatcacaggatacattgctaatggagagtgggaccttgtggaagttccaggtagacggaatgagaggttctacgac
tgctgcaaggagccgtacccagatgtgacttttacggtggtgatgcggagacgaacactgtattacggtcttaatctacttattccctgtg
tgctcatctctactctggccctgctggtgttcctgctgcctgctgactcaggagaaaagatctcgctcgggatcaccgtcctgctctcgct
gacagttttcatgcttttggtagcagagataatgccggcaacatctgactctgtgcctttaatagctcagtattttgccaccaccatggttatt
gttggactatctgtaatagccacagtctgggttttacaataccactaccatgatcctgaaggaggaaaaatgccaaaatggacacgtgtg
gtgctgttgaactggtgtgcctggttcctgcggatgaagagacctggcgaagacaaagttcgcccagcttgtcacaacaagcagccg
cgcagcagcctctccagcgtggatctaaacatcagccctggagtcgcacagtccaccaatggaaacctgctgtacatcggcttccgc
gggatggacactattcactatgctacttcacccgattctggggtgatctgcagccggcttgtggcgaccggagaggaggatgtgctgc
tccccggagcccaggcatcttcagtcagtagtagcggccctggagaaaccgaactgtccaagatcttggatgaggtgcggtacatttc
caagcgctttcgggaccaagacgaggaagacactgtgtgtaatgagtggaagtttgcagcgtctgtgattgaccgtctctgtctcatgg
ctttttccctgttcaccatcctctgcaccattggtatcttaatgtctgcccctaactttgttgaggccatatcaaaagattttttcacctga

(NCBI Accession No. NM_{—}201219; Zirger J. M. et al., Gen Expr. Patterns 3(6):747-754, 2003).

SEQ ID NO:17 is the amino acid sequence of the wild-type zebra fish α7 subunit, including amino acids 1-22 (underlined), which represent a signal peptide that is not present in the mature receptor subunit (the TM2 domain at amino acids 262-281 is also underlined for reference):

MGIWEYTLYFTTTCCLWSVSLQGEHQRRLYRDLMKDYNPLERPVFNDTHSLTVYFS
MSLMQIMDVDEKNQVLTTNIWLQLYWYDYYLQWNASEYPGVTNVRFPDSQIWKP
DILLYNSADERFDATFHTNVLVNSSGACQYLPPGIFKSTCYIDVRWFPFDLQRCDLKF
GSWTYGGWSLDLQMIDADITGYIANGEWDLVEVPGRRNERFYDCCKEPYPDVTFTV
VMRRRTLYYGLNLLIPCVLISTLALLVFLLPADSGEKISLGITVLLSLTVFMLLVAEIM
PATSDSVPLIAQYFATTMVIVGLSVIATVWVLQYHYHDPEGGKMPKWTRVVLLNW
CAWFLRMKRPGEDKVRPACHNKQPRSSLSSVDLNISPGVAQSTNGNLLYIGFRGMD
TIHYATSPDSGVICSRLVATGEEDVLLPGAQASSVSSSGPGETELSKILDEVRYISKRF
RDQDEEDTVCNEWKFAASVIDRLCLMAFSLFTILCTIGILMSAPNFVEAISKDFFT

SEQ ID NO:18 is a nucleotide coding sequence of the T6′S variant of the rat (and mouse) second transmembrane (TM2) domain from (the codon encoding serine, in place of threonine, at the sixth amino acid position of TM2 is underlined):

atctctcttggaatatctgtcttactttct

ctgactgtcttcatgctgcttgtggctgag

SEQ ID NO:119 is a nucleotide coding sequence of the T6′S variant α7 subunit, including nucleotides 1-66 (underlined), which encode a signal peptide that is not present in the mature receptor subunit (the TM2 domain at nucleotides 784-843 is underlined and the codon for the 6′ amino acid of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaatatctgtct
tactttctctgactgtcttcatgctgcttgtggctgagatcatgccagcaacatctgattctgtgcccttgatagcacaatacttcgccagca
ccatgatcatcgtgggcctctctgtagtggtgacagtgattgtgctgagatatcaccaccatgaccctgatggtggcaaaatgcctaagt
ggaccagaatcattctcctgaactggtgtgcatggtttctgcgcatgaagaggcccggagaggacaaggtgcggccagcttgtcagc
acaagcctcggcgctgcagcctggccagtgtggagctgagtgcaggtgctgggccacccaccagcaatggcaacctgctctacatt
ggcttccgaggcctggagggcatgcactgtgccccaactccagactctggggtcgtatgtggccgtttggcctgctccccaacacatg
atgagcacctcatgcacggtgcacacccctctgatggggaccccgacctggccaagatcctggaggaggtccgctacatcgccaac
cgcttccgctgccaggacgagagtgaggtgatctgcagtgaatggaagtttgcagcctgcgtggtggaccgcttgtgcctcatggcct
tttcggtctttaccatcatctgtaccatcggcatcctcatgtcagctccaaactttgtggaggctgtgtccaaagactttgcttaa

SEQ ID NO:20 is an amino acid sequence of the T6′S variant α7 subunit, including amino acids 1-22 (underlined), which represent a signal peptide that is not present in the mature receptor subunit (the TM2 domain at amino acids 262-281 is also underlined for reference):

MCGGRGGIWLALAAALLHVSLQGEFQRRLYKELVKNYNPLERPVANDSQPLTVYFS
LSLLQIMDVDEKNQVLTTNIWLQMSWTDHYLQWNMSEYPGVKNVRFPDGQIWKPD
ILLYNSADERFDATFHTNVLVNASGHCQYLPPGIFKSSCYIDVRWFPFDVQQCKLKF
GSWSYGGWSLDLQMQEADISSYIPNGEWDLMGIPGKRNEKFYECCKEPYPDVTYTV
TMRRRTLYYGLNLLIPCVLISALALLVFLLPADSGEKISLGISVLLSLTVFMLLVAEIM
PATSDSVPLIAQYFASTMIIVGLSVVVTVIVLRYHHHDPDGGKMPKWTRIILLNWCA
WFLRMKRPGEDKVRPACQHKPRRCSLASVELSAGAGPPTSNGNLLYIGFRGLEGMH
CAPTPDSGVVCGRLACSPTHDEHLMHGAHPSDGDPDLAKILEEVRYIANRFRCQDES
EVICSEWKFAACVVDRLCLMAFSVFTIICTIGILMSAPNFVEAVSKDFA

SEQ ID NO:21 is an example of a degenerate variant of a nucleotide sequence (SEQ ID NO:18) encoding the T6′S mutant second transmembrane (TM2) domain (SEQ ID NO:2), containing a degenerate codon (-tcc-) encoding serine at the sixth amino acid position of the TM2 domain:

atctctcttggaatatccgtcttactttct

ctgactgtcttcatgctgcttgtggctgag

SEQ ID NO:22 is an example of a degenerate variant of a nucleotide sequence (SEQ ID NO:18) encoding the T6′S mutant second transmembrane (TM2) domain (SEQ ID NO:2), containing a degenerate codon (-tca-) encoding serine at the sixth amino acid position of the TM2 domain:

atctctcttggaatatcagtcttactttct

ctgactgtcttcatgctgcttgtggctgag

SEQ ID NO:23 is an example of a degenerate variant of a nucleotide sequence (SEQ ID NO:18) encoding the T6′S mutant second transmembrane (TM2) domain (SEQ ID NO:2), containing a degenerate codon (-tcg-) encoding serine at the sixth amino acid position of the TM2 domain:

atctctcttggaatatcggtcttactttct

ctgactgtcttcatgctgcttgtggctgag

SEQ ID NO:24 is an example of a degenerate variant of a nucleotide sequence (SEQ ID NO:18) encoding the T6′S mutant second transmembrane (TM2) domain (SEQ ID NO:2), containing a degenerate codon (-agt-) encoding serine at the sixth amino acid position of the TM2 domain:

atctctcttggaataagtgtcttactttct

ctgactgtcttcatgctgcttgtggctgag

SEQ ID NO:25 is an example of a degenerate variant of a nucleotide sequence (SEQ ID NO:18) encoding the T6′S mutant second transmembrane (TM2) domain (SEQ ID NO:2), containing a degenerate codon (-agc-) encoding serine at the sixth amino acid position of the TM2 domain:

atctctcttggaataagcgtcttactttct

ctgactgtcttcatgctgcttgtggctgag

SEQ ID NO:26 is an example of a degenerate variant of a nucleotide sequence (SEQ ID NO:19) encoding a T6′S mutant α7 subunit (SEQ ID NO:20), containing a degenerate codon (-tcc-) encoding serine at the sixth amino acid position of the TM2 domain (the TM2 domain at nucleotides 784-843 is underlined and the codon for the 6′ amino acid of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaatatccgtc
ttactttctctgactgtcttcatgctgcttgtggctgagatcatgccagcaacatctgattctgtgcccttgatagcacaatacttcgccagc
accatgatcatcgtgggcctctctgtagtggtgacagtgattgtgctgagatatcaccaccatgaccctgatggtggcaaaatgcctaag
tggaccagaatcattctcctgaactggtgtgcatggtttctgcgcatgaagaggcccggagaggacaaggtgcggccagcttgtcag
cacaagcctcggcgctgcagcctggccagtgtggagctgagtgcaggtgctgggccacccaccagcaatggcaacctgctctacat
tggcttccgaggcctggagggcatgcactgtgccccaactccagactctggggtcgtatgtggccgtttggcctgctccccaacacat
gatgagcacctcatgcacggtgcacacccctctgatggggaccccgacctggccaagatcctggaggaggtccgctacatcgccaa
ccgcttccgctgccaggacgagagtgaggtgatctgcagtgaatggaagtttgcagcctgcgtggtggaccgcttgtgcctcatggcc
ttttcggtctttaccatcatctgtaccatcggcatcctcatgtcagctccaaactttgtggaggctgtgtccaaagactttgcttaa

SEQ ID NO:27 is an example of a degenerate variant of a nucleotide sequence (SEQ ID NO:19) encoding a T6′S mutant α7 subunit (SEQ ID NO:20), containing a degenerate codon (-tca-) encoding serine at the sixth amino acid position of the TM2 domain (the TM2 domain at nucleotides 784-843 is underlined and the codon for the 6′ amino acid of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaatatcagtc
ttactttctctgactgtcttcatgctgcttgtggctgagatcatgccagcaacatctgattctgtgcccttgatagcacaatacttcgccagc
accatgatcatcgtgggcctctctgtagtggtgacagtgattgtgctgagatatcaccaccatgaccctgatggtggcaaaatgcctaag
tggaccagaatcattctcctgaactggtgtgcatggtttctgcgcatgaagaggcccggagaggacaaggtgcggccagcttgtcag
cacaagcctcggcgctgcagcctggccagtgtggagctgagtgcaggtgctgggccacccaccagcaatggcaacctgctctacat
tggcttccgaggcctggagggcatgcactgtgccccaactccagactctggggtcgtatgtggccgtttggcctgctccccaacacat
gatgagcacctcatgcacggtgcacacccctctgatggggaccccgacctggccaagatcctggaggaggtccgctacatcgccaa
ccgcttccgctgccaggacgagagtgaggtgatctgcagtgaatggaagtttgcagcctgcgtggtggaccgcttgtgcctcatggcc
ttttcggtctttaccatcatctgtaccatcggcatcctcatgtcagctccaaactttgtggaggctgtgtccaaagactttgcttaa

SEQ ID NO:28 is an example of a degenerate variant of the nucleotide sequence (SEQ ID NO:119) encoding a T6′S mutant α7 subunit (SEQ ID NO:20), containing a degenerate codon (-tcg-) encoding serine at the sixth amino acid position of the TM2 domain (the TM2 domain at nucleotides 784-843 is underlined and the codon for the 6′ amino acid of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaatatcggtc
ttactttctctgactgtcttcatgctgcttgtggctgagatcatgccagcaacatctgattctgtgcccttgatagcacaatacttcgccagc
accatgatcatcgtgggcctctctgtagtggtgacagtgattgtgctgagatatcaccaccatgaccctgatggtggcaaaatgcctaag
tggaccagaatcattctcctgaactggtgtgcatggtttctgcgcatgaagaggcccggagaggacaaggtgcggccagcttgtcag
cacaagcctcggcgctgcagcctggccagtgtggagctgagtgcaggtgctgggccacccaccagcaatggcaacctgctctacat
tggcttccgaggcctggagggcatgcactgtgccccaactccagactctggggtcgtatgtggccgtttggcctgctccccaacacat
gatgagcacctcatgcacggtgcacacccctctgatggggaccccgacctggccaagatcctggaggaggtccgctacatcgccaa
ccgcttccgctgccaggacgagagtgaggtgatctgcagtgaatggaagtttgcagcctgcgtggtggaccgcttgtgcctcatggcc
ttttcggtctttaccatcatctgtaccatcggcatcctcatgtcagctccaaactttgtggaggctgtgtccaaagactttgcttaa

SEQ ID NO:29 is an example of a degenerate variant of the nucleotide sequence (SEQ ID NO:19) encoding a T6′S mutant α7 subunit (SEQ ID NO:20), containing a degenerate codon (-agt-) encoding serine at the sixth amino acid position of the TM2 domain (the TM2 domain at nucleotides 784-843 is underlined and the codon for the 6′ amino acid of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaataagtgtc
ttactttctctgactgtcttcatgctgcttgtggctgagatcatgccagcaacatctgattctgtgcccttgatagcacaatacttcgccagc
accatgatcatcgtgggcctctctgtagtggtgacagtgattgtgctgagatatcaccaccatgaccctgatggtggcaaaatgcctaag
tggaccagaatcattctcctgaactggtgtgcatggtttctgcgcatgaagaggcccggagaggacaaggtgcggccagcttgtcag
cacaagcctcggcgctgcagcctggccagtgtggagctgagtgcaggtgctgggccacccaccagcaatggcaacctgctctacat
tggcttccgaggcctggagggcatgcactgtgccccaactccagactctggggtcgtatgtggccgtttggcctgctccccaacacat
gatgagcacctcatgcacggtgcacacccctctgatggggaccccgacctggccaagatcctggaggaggtccgctacatcgccaa
ccgcttccgctgccaggacgagagtgaggtgatctgcagtgaatggaagtttgcagcctgcgtggtggaccgcttgtgcctcatggcc
ttttcggtctttaccatcatctgtaccatcggcatcctcatgtcagctccaaactttgtggaggctgtgtccaaagactttgcttaa

SEQ ID NO:30 is an example of a degenerate variant of the nucleotide sequence (SEQ ID NO:19) encoding a T6′S mutant α7 subunit (SEQ ID NO:20), containing a degenerate codon (-agc-) encoding serine at the sixth amino acid position of the TM2 domain (the TM2 domain at nucleotides 784-843 is underlined and the codon for the 6′ amino acid of TM2 (threonine) is bold for reference):

atgtgcggcgggcggggaggcatctggctggctctggccgcggcgctgctgcacgtgtccctgcaaggcgagttccagaggaggc
tgtacaaggagctggtcaagaactacaacccgctggagaggccggtggccaacgactcgcagccgctcaccgtgtacttctccctga
gtctcctgcagatcatggatgtggatgagaagaaccaagttttaaccaccaacatttggctacaaatgtcttggacagatcactatttgca
gtggaacatgtctgagtaccccggagtgaagaatgttcgttttccagatggccagatttggaaaccagacattctcctctataacagtgct
gatgagcgctttgatgccacgttccacaccaatgttttggtgaatgcatctgggcattgccagtatctccctccaggcatattcaagagct
cctgctacattgacgttcgctggttcccttttgatgtgcagcagtgcaaactgaagtttgggtcctggtcctatggagggtggtcactgga
cctgcaaatgcaagaggcagatatcagcagctatatccccaacggagaatgggatctcatgggaatccctggcaaaaggaatgaga
agttctatgagtgctgcaaagagccatacccagatgtcacctacacagtaaccatgcgccgtaggacactctactatggcctcaatctg
ctcatcccttgtgtactcatttcagccctggctctgctggtattcttgctgcctgcagactctggagagaaaatctctcttggaataagcgtc
ttactttctctgactgtcttcatgctgcttgtggctgagatcatgccagcaacatctgattctgtgcccttgatagcacaatacttcgccagc
accatgatcatcgtgggcctctctgtagtggtgacagtgattgtgctgagatatcaccaccatgaccctgatggtggcaaaatgcctaag
tggaccagaatcattctcctgaactggtgtgcatggtttctgcgcatgaagaggcccggagaggacaaggtgcggccagcttgtcag
cacaagcctcggcgctgcagcctggccagtgtggagctgagtgcaggtgctgggccacccaccagcaatggcaacctgctctacat
tggcttccgaggcctggagggcatgcactgtgccccaactccagactctggggtcgtatgtggccgtttggcctgctccccaacacat
gatgagcacctcatgcacggtgcacacccctctgatggggaccccgacctggccaagatcctggaggaggtccgctacatcgccaa
ccgcttccgctgccaggacgagagtgaggtgatctgcagtgaatggaagtttgcagcctgcgtggtggaccgcttgtgcctcatggcc
ttttcggtctttaccatcatctgtaccatcggcatcctcatgtcagctccaaactttgtggaggctgtgtccaaagactttgcttaa

SEQ ID NO:31 is a nucleotide coding sequence of the T6′S variant of the human second transmembrane (TM2) domain of (the codon encoding serine, in place of threonine, at the sixth amino acid position of TM2 is underlined):

atttccctggggatatctgtcttactctct

cttaccgtcttcatgctgctcgtggctgag

SEQ ID NO:32 is an example of a degenerate variant of a nucleotide sequence (SEQ ID NO:31) encoding the T6′S mutant second transmembrane (TM2) domain (SEQ ID NO:2), containing a degenerate codon (-tcc-) encoding serine at the sixth amino acid position of the TM2 domain:

atttccctggggatatccgtcttactctct

cttaccgtcttcatgctgctcgtggctgag

SEQ ID NO:33 is an example of a degenerate variant of a nucleotide sequence (SEQ ID NO:31) encoding the T6′S mutant second transmembrane (TM2) domain (SEQ ID NO:2), containing a degenerate codon (-tca-) encoding serine at the sixth amino acid position of the TM2 domain:

atttccctggggatatcagtcttactctct

cttaccgtcttcatgctgctcgtggctgag

SEQ ID NO:34 is an example of a degenerate variant of a nucleotide sequence (SEQ ID NO:31) encoding the T6′S mutant second transmembrane (TM2) domain (SEQ ID NO:2), containing a degenerate codon (-tcg-) encoding serine at the sixth amino acid position of the TM2 domain:

atttccctggggatatcggtcttactctct

cttaccgtcttcatgctgctcgtggctgag

SEQ ID NO:35 is an example of a degenerate variant of a nucleotide sequence (SEQ ID NO:31) encoding the T6′S mutant second transmembrane (TM2) domain (SEQ ID NO:2), containing a degenerate codon (-agt-) encoding serine at the sixth amino acid position of the TM2 domain:

atttccctggggataagtgtcttactctct

cttaccgtcttcatgctgctcgtggctgag

SEQ ID NO:36 is an example of a degenerate variant of a nucleotide sequence (SEQ ID NO:31) encoding the T6′S mutant second transmembrane (TM2) domain (SEQ ID NO:2), containing a degenerate codon (-agc-) encoding serine at the sixth amino acid position of the TM2 domain:

atttccctggggataagcgtcttactctct

cttaccgtcttcatgctgctcgtggctgag

SEQ ID NO:37 is a nucleotide coding sequence of the T6′S variant of the rhesus monkey second transmembrane (TM2) domain of (the codon encoding serine, in place of threonine, at the sixth amino acid position of TM2 is underlined):

atttccctggggatatctgtcttactctct

ctcactgtcttcatgctgctcgtggctgag

SEQ ID NO:38 is an example of a degenerate variant of a nucleotide sequence (SEQ ID NO:37) encoding the T6′S mutant second transmembrane (TM2) domain (SEQ ID NO:2), containing a degenerate codon (-tcc-) encoding serine at the sixth amino acid position of the TM2 domain:

atttccctggggatatccgtcttactctct

ctcactgtcttcatgctgctcgtggctgag

SEQ ID NO:39 is an example of a degenerate variant of a nucleotide sequence (SEQ ID NO:37) encoding the T6′S mutant second transmembrane (TM2) domain (SEQ ID NO:2), containing a degenerate codon (-tca-) encoding serine at the sixth amino acid position of the TM2 domain:

atttccctggggatatcagtcttactctct

ctcactgtcttcatgctgctcgtggctgag

SEQ ID NO:40 is an example of a degenerate variant of a nucleotide sequence (SEQ ID NO:37) encoding the T6′S mutant second transmembrane (TM2) domain (SEQ ID NO:2), containing a degenerate codon (-tcg-) encoding serine at the sixth amino acid position of the TM2 domain:

atttccctggggatatcggtcttactctct

ctcactgtcttcatgctgctcgtggctgag

SEQ ID NO:41 is an example of a degenerate variant of a nucleotide sequence (SEQ ID NO:37) encoding the T6′S mutant second transmembrane (TM2) domain (SEQ ID NO:2), containing a degenerate codon (-agt-) encoding serine at the sixth amino acid position of the TM2 domain:

atttccctggggataagtgtcttactctct

ctcactgtcttcatgctgctcgtggctgag

SEQ ID NO:42 is an example of a degenerate variant of a nucleotide sequence (SEQ ID NO:37) encoding the T6′S mutant second transmembrane (TM2) domain (SEQ ID NO:2), containing a degenerate codon (-agc-) encoding serine at the sixth amino acid position of the TM2 domain:

atttccctggggataagcgtcttactctct

ctcactgtcttcatgctgctcgtggctgag

SEQ ID NO:43 is a nucleotide coding sequence of the T6′S variant of the cow second transmembrane (TM2) domain of (the codon encoding serine, in place of threonine, at the sixth amino acid position of TM2 is underlined):

atctccctgggaatctctgttttactctcc

ctcaccgtcttcatgctgctggtagctgag

SEQ ID NO:44 is an example of a degenerate variant of a nucleotide sequence (SEQ ID NO:43) encoding the T6′S mutant second transmembrane (TM2) domain (SEQ ID NO:2), containing a degenerate codon (-tcc-) encoding serine at the sixth amino acid position of the TM2 domain:

atctccctgggaatctccgttttactctcc

ctcaccgtcttcatgctgctggtagctgag

SEQ ID NO:45 is an example of a degenerate variant of a nucleotide sequence (SEQ ID NO:43) encoding the T6′S mutant second transmembrane (TM2) domain (SEQ ID NO:2), containing a degenerate codon (-tca-) encoding serine at the sixth amino acid position of the TM2 domain:

atctccctgggaatctcagttttactctcc

ctcaccgtcttcatgctgctggtagctgag

SEQ ID NO:46 is an example of a degenerate variant of a nucleotide sequence (SEQ ID NO:43) encoding the T6′S mutant second transmembrane (TM2) domain (SEQ ID NO:2), containing a degenerate codon (-tcg-) encoding serine at the sixth amino acid position of the TM2 domain:

atctccctgggaatctcggttttactctcc

ctcaccgtcttcatgctgctggtagctgag

SEQ ID NO:47 is an example of a degenerate variant of a nucleotide sequence (SEQ ID NO:43) encoding the T6′S mutant second transmembrane (TM2) domain (SEQ ID NO:2), containing a degenerate codon (-agt-) encoding serine at the sixth amino acid position of the TM2 domain:

atctccctgggaatcagtgttttactctcc

ctcaccgtcttcatgctgctggtagctgag

SEQ ID NO:48 is an example of a degenerate variant of a nucleotide sequence (SEQ ID NO:43) encoding the T6′S mutant second transmembrane (TM2) domain (SEQ ID NO:2), containing a degenerate codon (-agc-) encoding serine at the sixth amino acid position of the TM2 domain:

atctccctgggaatcagcgttttactctcc

ctcaccgtcttcatgctgctggtagctgag

SEQ ID NO:49 is a nucleotide coding sequence of the T6′S variant of the chicken second transmembrane (TM2) domain of (the codon encoding serine, in place of threonine, at the sixth amino acid position of TM2 is underlined):

atctcactaggtatatctgttttattgtct

ctcaccgtcttcatgttactcgtggctgaa

SEQ ID NO:50 is an example of a degenerate variant of a nucleotide sequence (SEQ ID NO:49) encoding the T6′S mutant second transmembrane (TM2) domain (SEQ ID NO:2), containing a degenerate codon (-tcc-) encoding serine at the sixth amino acid position of the TM2 domain:

atctcactaggtatatccgttttattgtct

ctcaccgtcttcatgttactcgtggctgaa

SEQ ID NO:51 is an example of a degenerate variant of a nucleotide sequence (SEQ ID NO:49) encoding the T6′S mutant second transmembrane (TM2) domain (SEQ ID NO:2), containing a degenerate codon (-tca-) encoding serine at the sixth amino acid position of the TM2 domain:

atctcactaggtatatcagttttattgtct

ctcaccgtcttcatgttactcgtggctgaa

SEQ ID NO:52 is an example of a degenerate variant of a nucleotide sequence (SEQ ID NO:49) encoding the T6′S mutant second transmembrane (TM2) domain (SEQ ID NO:2), containing a degenerate codon (-tcg-) encoding serine at the sixth amino acid position of the TM2 domain:

atctcactaggtatatcggttttattgtct

ctcaccgtcttcatgttactcgtggctgaa

SEQ ID NO:53 is an example of a degenerate variant of a nucleotide sequence (SEQ ID NO:49) encoding the T6′S mutant second transmembrane (TM2) domain (SEQ ID NO:2), containing a degenerate codon (-agt-) encoding serine at the sixth amino acid position of the TM2 domain:

atctcactaggtataagtgttttattgtct

ctcaccgtcttcatgttactcgtggctgaa

SEQ ID NO:54 is an example of a degenerate variant of a nucleotide sequence (SEQ ID NO:49) encoding the T6′S mutant second transmembrane (TM2) domain (SEQ ID NO:2), containing a degenerate codon (-agc-) encoding serine at the sixth amino acid position of the TM2 domain:

atctcactaggtataagcgttttattgtct

ctcaccgtcttcatgttactcgtggctgaa

SEQ ID NO:55 is a nucleotide coding sequence of the T6′S variant of the zebra-fish second transmembrane (TM2) domain of (the codon encoding serine, in place of threonine, at the sixth amino acid position of TM2 is underlined):

atctcgctcgggatctctgtcctgctctcg

ctgacagttttcatgcttttggtagcagag

SEQ ID NO:56 is an example of a degenerate variant of a nucleotide sequence (SEQ ID NO:55) encoding the T6′S mutant second transmembrane (TM2) domain (SEQ ID NO:2), containing a degenerate codon (-tcc-) encoding serine at the sixth amino acid position of the TM2 domain:

atctcgctcgggatctccgtcctgctctcg

ctgacagttttcatgcttttggtagcagag

SEQ ID NO:57 is an example of a degenerate variant of a nucleotide sequence (SEQ ID NO:55) encoding the T6′S mutant second transmembrane (TM2) domain (SEQ ID NO:2), containing a degenerate codon (-tca-) encoding serine at the sixth amino acid position of the TM2 domain:

atctcgctcgggatctcagtcctgctctcg

ctgacagttttcatgcttttggtagcagag

SEQ ID NO:58 is an example of a degenerate variant of a nucleotide sequence (SEQ ID NO:55) encoding the T6′S mutant second transmembrane (TM2) domain (SEQ ID NO:2), containing a degenerate codon (-tcg-) encoding serine at the sixth amino acid position of the TM2 domain:

atctcgctcgggatctcggtcctgctctcg

ctgacagttttcatgcttttggtagcagag

SEQ ID NO:59 is an example of a degenerate variant of a nucleotide sequence (SEQ ID NO:55) encoding the T6′S mutant second transmembrane (TM2) domain (SEQ ID NO:2), containing a degenerate codon (-agt-) encoding serine at the sixth amino acid position of the TM2 domain:

atctcgctcgggatcagtgtcctgctctcg

ctgacagttttcatgcttttggtagcagag

SEQ ID NO:60 is an example of a degenerate variant of a nucleotide sequence (SEQ ID NO:55) encoding the T6′S mutant second transmembrane (TM2) domain (SEQ ID NO:2), containing a degenerate codon (-agc-) encoding serine at the sixth amino acid position of the TM2 domain:

atctcgctcgggatcagcgtcctgctctcg

ctgacagttttcatgcttttggtagcagag

### DETAILED DISCLOSURE OF THE INVENTION

The T6′S variant of the α7 nicotinic receptor subunit contains a T→S substitution at the 6′ position of the second transmembrane (TM2) domain (corresponding to residue 244 of the wild-type rat α7 subunit). The biophysical and pharmacological properties of the T6′S variant, including its slow rate of desensitization, make it particularly useful for drug screening.

The term “AChR”, as used herein, refers to a receptor for the neurotransmitter acetylcholine (“Ach”). AChRs are broadly subclassified as nicotinic or muscarinic. These types differ in their pharmacology, structures, and signal transduction mechanisms.

The term “nAChR”, as used herein, refers to a nicotinic acetylcholine receptor. Although nAChRs of various subunit structures are best known in muscle cells, neurons, and chromaffin cells, they are not necessarily excluded from other cells types (e.g., glial cells, mast cells, blood cells, fibroblasts, etc.).

The term “nAChR subunit”, as used herein, refers to a proteinaceous molecule that can combine with other such molecules in the formation of a nAChR. For example, the muscle nAChR is believed to be a pentamer comprised of four types of transmembrane subunit: two α1 subunits, one β1 subunit, one δ subunit and one γ or ε subunit depending upon the nAChR form. Neuronal nAChR analogously are also thought to be pentameric and comprised of related but different subunits. At present, eight neuronal α subunits (α2-α9) and three neuronal β subunits (β2-β4) have been isolated. Some neuronal nAChRs appear to require at least one α subunit and at least one β subunit for a functional complex (e.g., exhibiting ion channel response to ACh or other agonists). Some subunits, however, may assemble to form “homooligomeric” nAChR, as in the case of α7 nAChR in Xenopus oocytes and in transfected mammalian cells. Although the combination of nAChR subunits with subunits related to other types of receptor (e.g., other classes of ligand-gated ion channel) has not been demonstrated, such combinations are possible and contemplated within the scope of the present invention.

The term “wild-type” (WT), as used herein, refers to the typical, most common or conventional form as it occurs in nature. The human wild-type α7 nAChR was described by Doucette-Stamm et al. (Drug Dev. Res. 30: 252-256, 1993; and U.S. Pat. No. 5,837,489, which are incorporated herein by reference in their entireties). The rat and mouse wild-type α7 nAChRs were described by Khiroug, S. S., et al. (J. Physiol. (Lond.) 540 (Pt 2): 425-434, 2002) (NCBI Accession No. L31629); and Orr-Urtreger, A. et al. (Genomics 26 (2): 399-402, 1995) (NCBI Accession No. L37663), respectively. An abbreviation of the form “α7XnnnO” intends an α7 subunit in which the amino acid X, located at position nnn, relative to the wild-type sequence, has been replaced by amino acid O. Thus, for example, the α7T144S subunit indicates the threonine residue located at position 144 in the wild-type receptor has been replaced by a serine residue.

The term “nicotinic cholinergic agonist”, as used herein, refers to an agent that binds to and activates a nicotinic acetylcholine receptor. By “activates” is intended the elicitation of one or more pharmacological, physiological, or electrophysiological responses. Such a response includes, but is not limited to, cell membrane depolarization and increased permeability to Ca₂⁺ other cations.

The term “nicotinic cholinergic antagonist”, as used herein, refers to a substance that binds to a nicotinic acetylcholine receptor and prevents agonists from activating the receptor. Pure antagonists do not activate the receptor, but some substances may have mixed agonist and antagonist properties. Nicotinic cholinergic channel blockers block the ability of agonists to elicit current flow through the nicotinic acetylcholine receptor channel, but do so by blocking the channel rather than by preventing agonists from binding to and activating the receptor.

The term “nicotinic cholinergic modulator”, as used herein, refers to an agent that influences the activity of the nicotinic acetylcholine receptor through interaction at one or more sites other than the classic agonist binding site. The modulator may itself increase or decrease receptor activity, or may influence agonist activity (for example, potentiating responses) without itself eliciting an overt change in channel current. A single agent can have different properties at different nicotinic acetylcholine receptor subtypes, for example, being an agonist at one receptor and antagonist at another, or an antagonist at one and a channel blocker at another.

The term “nAChR regulator”, as used herein, refers to an agent that may act as an agonist, antagonist, channel blocker or modulator.

The term “polynucleotide”, as used herein, refers to a polymeric form of nucleotides of any length, either ribonucleotides or deoxyribonucleotides. This term refers only to the primary structure of the molecule. Thus, the term includes double-stranded and single-stranded DNA, as well as double-stranded and single-stranded RNA. It also includes modifications, such as by methylation and/or by capping, and unmodified forms of the polynucleotide.

The term “variant”, as used herein, refers to an oligonucleotide sequence that differs from the related wild-type sequence in one or more nucleotides. Such a variant oligonucleotide is expressed as a protein variant which, as used herein, indicates a polypeptide sequence that differs from the wild-type polypeptide in the substitution, insertion or deletion of one or more amino acids. The variant polypeptide differs in primary structure (amino acid sequence), but may or may not differ significantly in secondary or tertiary structure or in function relative to the wild-type.

The term “mutant”, as used herein, generally refers to an organism or a cell displaying a new genetic character or phenotype as the result of change in its gene or chromosome. In some instances, however, the term “mutant” may be used in reference to a variant protein or oligonucleotide and “mutation” may refer to the change underlying the variant.

The terms “polypeptide” and “protein” are used interchangeably herein and indicate a molecular chain of amino acids of any length linked through peptide bonds. Thus, peptides, oligopeptides, and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

The polypeptides of the present invention are, optionally, N-acetylated and/or C-terminal amidated. The polypeptides of the present invention may be synthetically produced using methods well known in the art utilizing D or L isomeric amino acid precursors. Therapeutic peptides of the present invention preferably contain one, a few, or all of their amino acid residues in the D-isomer conformation. The polypeptides of the present invention may also be produced by recombinant technology, as small polypeptides, as small synthetic fusion proteins comprising one or more polypeptides of the present invention fused to a targeting polypeptide. The term “fusion protein” as used herein refers to protein constructs that are the result of combining multiple protein domains or linker regions for the purpose of gaining function of the combined functions of the domains or linker regions. This is most often accomplished by molecular cloning of the nucleotide sequences to result in the creation of a new polynucleotide sequence that codes for the desired protein. Alternatively, creation of a fusion protein may be accomplished by chemically joining two proteins together.

Unless otherwise specified, the terms “T6′S variant” or “T6′S mutant” are used herein interchangeably to refer a TM2 domain having a serine residue in the sixth amino acid position (6′S), an α7 subunit polypeptide having a TM2 domain with a serine in the 6′S position, and/or an nACh receptor having a TM2 domain with a serine in the 6′S position, functional fragments or variants of any of the foregoing polypeptides; or polynucleotides encoding any of the foregoing polypeptides, including functional fragments or variants thereof. Preferably, the TM2 domain is at least 20 amino acids in length. Preferably, the TM2 domain comprises the amino acid sequence of ISLGISVLLSLTVFMLLVAE (SEQ ID NO:2). The terms “T6′S variant” or “T6′S mutant” are used herein interchangeably to refer to any polynucleotide encoding an amino acid sequence comprising a TM2 domain having a serine residue in the sixth amino acid position (6′S). Preferably, the polynucleotide encodes the amino acid sequence of ISLGISVLLSLTVFMLLVAE (SEQ ID NO:2), such as the nucleic acid sequences of SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants (i.e., structurally conservative mutants) of these nucleic acid sequences.

The nucleotide sequences encoding the T6′S variant, or encoding the T6′S variant nAChR used in the subject invention include “homologous” or “modified” nucleotide sequences. Homologous nucleic acid sequences will be understood to include any nucleotide sequence obtainable by mutagenesis according to techniques well known to persons skilled in the art, and exhibiting modifications in relation to the normal sequences. For example, mutations in the regulatory and/or promoter sequences for the expression of a polypeptide that result in a modification of the level of expression of a polypeptide according to the invention provide for a “modified nucleotide sequence”. Likewise, substitutions, deletions, or additions of nucleic acid to the polynucleotides of the invention provide for “homologous” or “modified” nucleotide sequences. In various embodiments, “homologous” or “modified” nucleic acid sequences have substantially the same biological function in vitro and/or in vivo as the T6′S variant of the present invention. The function of homologues of the invention can be assessed by a number of methods known for assessing nAChR receptor function known in the art, including those disclosed herein. For example, function of homologues can be determined in the Xenopus oocyte by a variety of electrophysiological techniques including intracellular voltage recording, two-electrode voltage clamp, patch clamp methods, and the like. A “homologous” or “modified” nucleotide sequence will also be understood to include a splice variant of the polynucleotides of the instant invention or any nucleotide sequence encoding a “modified polypeptide” as defined below. A “homologue” or “modified” nucleotide or amino acid sequence will also be understood to include mammalian homologues of the nucleotide sequences and amino acid disclosed herein.

Homologues of the present invention include those sequences having “functionally conservative mutations” and/or “structurally conservative mutations.” The phrase “functionally conservative mutation”, as used herein, intends a change in a polynucleotide encoding a derivative polypeptide in which the activity is not substantially altered compared to that of the polypeptide from which the derivative is made. Such derivatives may have, for example, amino acid insertions, deletions, or substitutions in the relevant molecule that do not substantially affect its properties (see Table 1). Functionally conservative mutants of the amino acid sequence of SEQ ID NO:2, SEQ ID NO:20, amino acids 23 to 502 of SEQ ID NO:20, and other T6′S polypeptides disclosed herein, are encompassed by the present invention.

The phrase “structurally conservative mutant”, as used herein, refers to a polynucleotide containing changes in the nucleic acid sequence but encoding a polypeptide having the same amino acid sequence as the polypeptide encoded by the polynucleotide from which the degenerate variant is derived. This can occur because a specific amino acid may be encoded by more than one codon (sequence of three nucleotides). Structurally conservative mutants of the nucleic acid sequences encoding SEQ ID NO: 2, SEQ ID NO:20, amino acids 23 to 502 of SEQ ID NO:20, and other T6′S polypeptides disclosed herein are encompassed by the present invention. For example, degenerate variants of the T6′S TM2 domain (SEQ ID NO:2) having alternative codons that encode serine at the sixth amino acid position are SEQ ID NO:21, SEQ ID NO:22, SEQ ID NO:23, SEQ ID NO:24, and SEQ ID NO:25. Degenerate codons encoding serine at the sixth amino acid position include -tct-, -tcc-, -tca-, -tcg-, -agt-, and -agc-. Examples of degenerate variants of the T6′S α7 receptor having alternative codons that encode serine at the sixth amino acid position of the TM2 domain include, but are not limited to, SEQ ID NO:26, SEQ ID NO:27, SEQ ID NO:28, SEQ ID NO:29, and SEQ ID NO:30. Specific embodiments of these degenerate variants of the T6′S α7 receptor are nucleotides 67 to 1509 of SEQ ID NO:26, nucleotides 67 to 1509 of SEQ ID NO:27, nucleotides 67 to 1509 of SEQ ID NO:28, nucleotides 67 to 1509 of SEQ ID NO:29, nucleotides 67 to 1509 of SEQ ID NO:30, and the nucleotide sequences of SEQ ID NOs. 31 to 60. Of course, the polynucleotides of the present invention may include further degenerate codons as well (i.e., in addition to those codons encoding serine in the sixth amino acid position of the TM2 domain).

Accordingly, the polynucleotides of the present include degenerate variants which, because of the degeneracy of the genetic code, have different nucleic acid sequences (i.e., one or more differing codons) from those polynucleotides exemplified herein, but which nonetheless encode the same amino acid sequences. A listing of valid codons for any given amino acid can be readily determined by those having knowledge of, or access to, the genetic code. Alternatively, so-called “back-translation” tools can be utilized to reverse-translate any given amino acid into its respective codons. One such tool is the “Utilities/Codon Calculator” feature of DNATOOLS for WINDOWS 95/98/NT/2000, which displays all possible degenerate codons for each amino acid input.

A homologous nucleotide sequence, for the purposes of the present invention, encompasses a nucleotide sequence having a percentage identity with the bases of the nucleotide sequences of between at least (or at least about) 20.00% to 99.99% (inclusive). The aforementioned range of percent identity is to be taken as including, and providing written description and support for, any fractional percentage, in intervals of 0.01%, between 20.00% and 99.99%. These percentages are purely statistical and differences between two nucleic acid sequences can be distributed randomly and over the entire sequence length.

In various embodiments, homologous sequences exhibiting a percentage identity with the bases of the nucleotide sequences of the present invention can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polynucleotide sequences of the instant invention. Homologous nucleotide and amino acid sequences include mammalian homologues of the rat T6′S variant and mammalian homologues of the rat T6′S variant nAChR. In a specific embodiment, the mammalian homologue is a nucleotide sequence encoding the human T6′S variant or the human T6′S variant nAChR. In another embodiment, the mammalian homologue is the amino acid sequence of the human T6′S variant or the human T6′S variant nAChR.

The T6′S variant homologues of the present invention include polypeptides containing, as a primary amino acid sequence, all or part of an exemplified T6′S variant polypeptide sequence. The T6′S variant homologues thus include T6′S variant polypeptides having conservative substitutions, i.e., altered sequences in which functionally equivalent amino acid residues are substituted for residues within the sequence resulting in a peptide which is biologically active. For example, one or more amino acid residues within the sequence can be substituted by another amino acid of a similar polarity which acts as a functional equivalent, resulting in a silent alteration. In one aspect of the present invention, conservative substitutions for an amino acid within the sequence may be selected from other members of the class to which the amino acid belongs (see Table 1). Conservative substitutions also include substitutions by amino acids having chemically modified side chains that do not eliminate the biological function of the resulting T6′S variant homologue.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

Both protein and nucleic acid sequence homologies may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman Proc. Natl. Acad. Sci. USA, 1988, 85(8):2444-2448; Altschul et al. J. Mol. Biol., 1990, 215(3):403-410; Thompson et al. Nucleic Acids Res., 1994, 22(2):4673-4680; Higgins et al. Methods Enzymol., 1996, 266:383-402; Altschul et al. J. Mol. Biol., 1990, 215(3):403-410; Altschul et al. Nature Genetics, 1993, 3:266-272).

Identity and similarity of related nucleic acid molecules and polypeptides can be readily calculated by known methods. Such methods include, but are not limited to, those described in Computational Molecular Biology, Lesk, A. M., ed., Oxford University Press, New York, 1988; York (1988); Biocomputing: Informatics and Genome Projects, Smith, D. W., ed., Academic Press, New York, 1993; York (1993); Computer Analysis of Sequence Data, Part 1, Griffin, A. M., and Griffin, H. G., eds., Humana Press, New Jersey, 1994; Jersey (1994); Sequence Analysis in Molecular Biology, von Heinje, G., Academic Press, 1987; Press (1987); Sequence Analysis Primer, Gribskov, M. and Devereux, J., eds., M. Stockton Press, New York, 1991; York (1991); and Carillo et al., SIAM J. Applied Math., 48:1073 (1988).

The methods, vectors, and compositions of the present invention can utilize functional fragments of nucleic acid sequences encoding the T6′S variant or T6′S variant nAChR disclosed herein, so long as the functional fragments include those nucleotides encoding the second transmembrane (TM2) domain of the α7 subunit and have the T→S substitution in the sixth amino acid position of the TM2 domain.

Representative fragments of the nucleotide sequences according to the invention will be understood to mean any polynucleotide fragment having at least 30 consecutive nucleotides, preferably at least 35 consecutive nucleotides, and still more preferably at least 40 or at least 50 consecutive nucleotides of the sequence from which it is derived. The upper limit for such fragments is one nucleotide less than the total number of nucleotides found in the full-length sequence (or, in certain embodiments, of the full length open reading frame (ORF) identified herein).

In other embodiments, depending upon the relevant full-length sequence from which they are derived, fragments can comprise consecutive nucleotides of 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, and up to one nucleotide less than the full-length T6′S variant TM2 domain, up to one nucleotide less than the full-length T6′S variant subunit, or up to one nucleotide less than the full-length T6′S variant nAChR.

It is also well known in the art that restriction enzymes can be used to obtain functional fragments of the nucleic acid sequences. For example, Bal31 exonuclease can be conveniently used for time-controlled limited digestion of DNA (commonly referred to as “erase-a-base” procedures). See, for example, Maniatis et al. Molecular Cloning: A Laboratory Manual, 1982, Cold Spring Harbor Laboratory, New York; Wei et al. J. Biol. Chem., 1983, 258:13006-13512.

The methods, vectors, and compositions of the present invention can utilize amino acid sequences that are functional fragments of the full-length T6′S variant TM2 domain, the T6′S variant subunit, or T6′S variant nAChR.

Representative fragments of the polypeptides according to the invention will be understood to mean any polypeptide fragment having at least 9 or 10 consecutive amino acids, preferably at least 15 amino acids, and still more preferably at least 20 or at least 30 consecutive amino acids of the polypeptide sequence from which it is derived. The upper limit for such fragments is one amino acid less than the total number of amino acids found in the full-length sequence.

In other embodiments, depending upon the relevant full-length sequence from which they are derived, fragments of the polypeptides can comprise consecutive amino acids of 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, and up to one amino acid less than the full-length T6′S variant TM2 domain, up to one amino acid less than the full-length T6′S variant subunit, or up to one amino acid less than the full-length T6′S variant nAChR. Fragments of polypeptides can be any portion of the full-length amino acid sequence (including human or non-human mammalian homologues) that exhibit functional activity, e.g., a C-terminally or N-terminally truncated version, or an intervening portion of the full-length sequence.

The terms “recombinant host cells”, “host cells”, “cells”, “cell lines”, “cell cultures”, and other such terms denoting microorganisms or higher eukaryotic cell lines cultured as unicellular entities refer to cells which can be, or have been, used as recipients for recombinant vectors or other transfer DNA, immaterial of the method by which the DNA is introduced into the cell or the subsequent disposition of the cell. The terms include the progeny of the original cell that has been transfected. Cells in primary culture as well as cells such as oocytes also can be used as recipients.

The term “genetic modification” as used herein refers to the stable or transient alteration of the genotype of a cell of the subject invention by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides in addition to those encoding the T6′S variant TM2 domain, the T6′S variant α7 subunit, or T6′S variant nAChR. A translation initiation codon can be inserted as necessary, making methionine the first amino acid in the sequence.

The term “vector” is used to refer to any molecule (e.g., nucleic acid, plasmid, or virus) usable to transfer coding sequence information (e.g., nucleic acid sequence encoding the T6′S variant TM2 domain, the T6′S variant α7 subunit, or T6′S variant nACh receptor), such as to a host cell. A vector typically includes a replicon in which another polynucleotide segment is attached, such as to bring about the replication and/or expression of the attached segment. The term includes expression vectors, cloning vectors, and the like. Thus, the term includes gene expression vectors capable of delivery/transfer of exogenous nucleic acid sequences into a host cell. The term “expression vector” refers to a vector that is suitable for use in a host cell (e.g., a patient's cell, tissue culture cell, cells of a cell line, etc.) and contains nucleic acid sequences which direct and/or control the expression of exogenous nucleic acid sequences. Expression includes, but is not limited to, processes such as transcription, translation, and RNA splicing, if introns are present. Nucleic acid sequences can be modified according to methods known in the art to provide optimal codon usage for expression in a particular expression system. The vector may include elements to control targeting, expression and transcription of the nucleic acid sequence in a cell selective manner as is known in the art. It should be noted that often the 5′UTR and/or 3′UTR of the gene may be replaced by the 5′UTR and/or 3′UTR of the expression vehicle. The vector can include a promoter for controlling transcription of the exogenous material and can be either a constitutive or inducible promoter to allow selective transcription. The expression vector can also include a selection gene.

A “coding sequence” is a polynucleotide sequence that is transcribed into mRNA and/or translated into a polypeptide. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences. Variants or analogs may be prepared by the deletion of a portion of the coding sequence, by insertion of a sequence, and/or by substitution of one or more nucleotides within the sequence. Techniques for modifying nucleotide sequences, such as site-directed mutagenesis, are well known to those skilled in the art (See, e.g., Sambrook et al., Molecular Cloning: A Laboratory Manual, Second Edition, 1989; DNA Cloning, Vols. I and II, D. N. Glover ed., 1985). Optionally, the polynucleotides of the present invention, and composition and methods of the invention that utilize such polynucleotides, can include non-coding sequences.

The term “operably-linked” is used herein to refer to an arrangement of flanking control sequences wherein the flanking sequences so described are configured or assembled so as to perform their usual function. Thus, a flanking control sequence operably-linked to a coding sequence may be capable of effecting the replication, transcription and/or translation of the coding sequence under conditions compatible with the control sequences. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a polypeptide will typically have its own operably-linked promoter sequence.

The terms “transfection” and “transformation” are used interchangeably herein to refer to the insertion of an exogenous polynucleotide into a host cell, irrespective of the method used for the insertion, the molecular form of the polynucleotide that is inserted, or the nature of the cell (e.g., prokaryotic or eukaryotic). The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome.

The term “isolated”, as used herein, when referring to a polynucleotide or a polypeptide, means that the indicated molecule is present in the substantial absence of other similar biological macromolecules. The term “isolated” as used herein means that at least 75 wt. %, more preferably at least 85 wt. %, more preferably still at least 95 wt. %., and most preferably at least 98 wt. % of a composition is the isolated polynucleotide or polypeptide. An “isolated polynucleotide” that encodes a particular polypeptide refers to a polynucleotide that is substantially free of other nucleic acid molecules that do not encode the subject polypeptide; however, the molecule may include functionally and/or structurally conservative mutations as defined herein.

The term “test sample”, as used herein, refers to biological samples and non-biological samples (e.g., synthetic fluids). Biological samples which can be evaluated by the methods of the present invention described herein include body fluids such as whole blood, tissues and cell preparations.

The terms “cell” and “cells” are used interchangeably herein to refer to a single cell or plurality of cells (i.e., at least one cell).

The following single-letter amino acid abbreviations are used throughout the text: Alanine: A; Arginine: R; Asparagine: N; Aspartic acid: D; Cysteine: C; Glutamine: Q; Glutamic acid: E; Glycine: G; Histidine: H; Isoleucine: I; Leucine: L; Lysine: K; Methionine: M; Phenylalanine: F; Proline: P; Serine: S; Threonine: T; Tryptophan: W; Tyrosine: Y; and Valine: V.

The T6′S variant TM2 domain, the T6′S variant α7 subunit, the T6′S variant nACh receptor; polynucleotides encoding the T6′S variant TM2 domain, the T6′S variant α7 subunit, and the T6′S variant nACh receptor; and methods of making the T6′S variant TM2 domain, the T6′S variant α7 subunit, and the T6′S variant nACh receptor, are provided herein, and encompassed by the subject invention. The invention includes not only the T6′S variant but also methods for screening compounds using the T6′S variant and cells expressing the T6′S variant TM2 domain. Further, polynucleotides and antibodies which can be used in methods for detection of the variant subunit, as well as the reagents useful in these methods, are provided. Compounds and polynucleotides useful in regulating the variant and its expression also are provided as disclosed herein below.

Accordingly, one aspect of the present invention is an isolated polynucleotide that encodes a variant of the nAChR TM2 domain of the α7 subunit, wherein the variant of the nAChR TM2 domain comprises the amino acid sequence of SEQ ID NO:2, or a functional fragment or homologue thereof, wherein the functional fragment or homologue contains the T→S substitution at the T6′S position. In one embodiment, the polynucleotide comprises the nucleic acid sequence of SEQ ID NO:18. In other embodiments, the polynucleotide comprises the nucleic acid sequence of SEQ ID NO:21, SEQ ID NO:22, SEQ ID NO:23, SEQ ID NO:24, or SEQ ID NO:25, which are each a degenerate variant of SEQ ID NO:18.

In other embodiments, the polynucleotides of the present invention comprise the nucleic acid coding sequences of the TM2 domains of human, mouse, rat, rhesus monkey, domestic cow, chicken, or zebrafish, or mammalian or non-mammalian homologs of these polynucleotides, wherein the codon encoding the sixth amino acid residue of the TM2 domain has been altered to encode serine. Preferably, the altered TM2 domain comprises SEQ ID NO:2. In other embodiments, the polynucleotides of the present invention comprise the coding sequence of the nAChR α7 subunit of human, mouse, rat, rhesus monkey, domestic cow, chicken, or zebrafish, or mammalian or non-mammalian homologs of these polynucleotides, wherein the codon encoding the sixth acid residue of the TM2 domain has been altered to encode serine. Preferably, the altered TM2 domain comprises SEQ ID NO:2.

The present invention further includes an isolated polynucleotide that encodes a variant of the wild-type nAChR α7 subunit, such as the wild-type nAChR α7 subunit of rat, human, mouse, monkey, cow, chicken, and zebrafish (the coding sequences of SEQ ID NO:4, SEQ ID NO:6, SEQ ID NO:8, SEQ ID NO:10, SEQ ID NO:12, SEQ ID NO:14, and SEQ ID NO:16).

In another aspect, the present invention includes an isolated polypeptide that is a variant of the wild-type TM2 domain of the α7 subunit, wherein the domain comprises the amino acid sequence of SEQ ID NO:2.

In other embodiments, the polypeptides of the present invention comprise the amino acid sequences of the mature α7 subunit of human, mouse, rat, rhesus monkey, domestic cow, chicken, or zebrafish (i.e., comprising the amino acid sequences of SEQ ID NO: 5, SEQ ID NO:7, SEQ ID NO:9, SEQ ID NO:11, SEQ ID NO:13, SEQ ID NO:15, or SEQ ID NO:17, with or without signal peptides), or mammalian or non-mammalian homologs of these polypeptides that have a TM2 domain, wherein the sixth amino acid residue of the TM2 domain in the amino acid sequence has been altered to encode serine. Preferably, the altered TM2 domain comprises SEQ ID NO:2. In a specific embodiment, the sixth amino acid is altered from a threonine to a serine (T→S).

A polynucleotide encoding the T6′S variant TM2 domain, the T6′S variant α7 subunit, or the T6′S variant nACh receptor can be derived from genomic or cDNA as a starting material, prepared by synthesis, or by a combination of techniques. The polynucleotide can then be used to express the T6′S variant TM2 domain, the T6′S variant α7 subunit, or the T6′S variant nACh receptor, or as a template for the preparation of RNA using methods well known in the art (see, Sambrook et al., Molecular Cloning: A Laboratory Manual, Second Edition, 1989).

One method for obtaining the desired polynucleotide involves isolating cDNA encoding the wild-type α7 nAChR subunit as described by Doucette-Stamm et al. Drug Dev. Res., 1993, 30: 252-256. The wild-type cDNA thus obtained can then be modified by site-directed mutagenesis using, for example QUICKCHANGE kits (STRATAGENE, LaJolla, Calif.) and amplified using the polymerase chain reaction (“PCR”) and primer sequences designed to obtain the DNA encoding the T6′S variant.

PCR employs short oligonucleotide primers (generally 10-20 nucleotides in length) that match opposite ends of a desired sequence within the wild-type DNA molecule. The sequence between the primers need not be known. The initial template can be either RNA or DNA. If RNA is used, it is first reverse-transcribed to cDNA. The cDNA is then denatured, using well known techniques such as heat, and appropriate oligonucleotide primers are added in molar excess.

The primer can be made specific by keeping primer length and base composition within relatively narrow limits, and by keeping the mutant base corresponding to T6′S centrally located (Zoler et al. (1983) Meth. Enzymol. 100:468). Primer extension is effected using DNA polymerase in the presence of deoxynucleotide triphosphates or nucleotide analogs. The resulting product includes the respective primers at their 5′-termini, covalently linked to the newly synthesized complements of the original strands. The replicated molecule is again denatured, hybridized with primers, and so on, until the product is sufficiently amplified. Such PCR methods are described in the art, e.g., U.S. Pat. Nos. 4,965,188; 4,800,159; 4,683,202; 4,683,195; incorporated herein by reference in their entireties. The product of the PCR is cloned and the clones containing the mutated DNA, derived by segregation of the primer extended strand, selected. Selection can be accomplished using the mutant primer as a hybridization probe.

By way of example, two complimentary olignonucleotides can be synthesized which contain the desired mutation flanked by 10-15 bases of unmodified nucleotide sequence. Using a thermal cycler, Pfu DNA polymerase extends the sequence around the whole vector, generating a plasmid with staggered nicks. Each cycle builds only off the parent strands, and therefore there is no amplification of misincorporations. After approximately 12-16 cycles, the product can then be treated with the restriction endonuclease Dpn I, which digests the methylated parent DNA into numerous small pieces. Optionally, the product can then be transformed into cells, such as E. coli, which repair the nicks. After linearization and purification of cloned cDNAs, RNA transcripts can be prepared in vitro using the appropriate mMessage mMachine kit from AMBION Inc. (Austin, Tex.).

Alternatively, the wild-type DNA may be obtained from an appropriate DNA library. DNA libraries may be probed using the procedure described by Grunstein et al. (Proc. Natl. Acad. Sci. USA 73:3961, 1975).

Synthetic oligonucleotides may be prepared using an automated oligonucleotide synthesizer such as that described by Warner (DNA 3:401, 1984). If desired, the synthetic strands may be labeled with ³²P by treatment with polynucleotide kinase in the presence of ₃₂P-ATP, using standard conditions for the reaction. DNA sequences, including those isolated from genomic or cDNA libraries, may be modified by known methods which include site-directed mutagenesis as described by Zoller (Nucleic Acids Res. 10:6487, 1982). Briefly, the DNA to be modified is packaged into phage as a single stranded sequence, and converted to a double stranded DNA with DNA polymerase using, as a primer, a synthetic oligonucleotide complementary to the portion of the DNA to be modified, and having the desired modification included in its own sequence. Culture of the transformed bacteria, which contain replications of each strand of the phage, are plated in agar to obtain plaques. Theoretically, 50% of the new plaques contain phage having the mutated sequence, and the remaining 50% have the original sequence. Replicates of the plaques are hybridized to labeled synthetic probe at temperatures and conditions suitable for hybridization with the correct strand, but not with the unmodified sequence. The sequences which have been identified by hybridization are recovered and cloned. Alternatively, it may be necessary to identify clones by sequence analysis if there is difficulty in distinguishing the variant from wild-type by hybridization. In any case, the DNA can be sequence-confirmed.

Once produced, the DNA may then be incorporated into a cloning vector or an expression vector for replication in a suitable host cell. Vector construction employs methods known in the art. Generally, site-specific DNA cleavage is performed by treating with suitable restriction enzymes under conditions which generally are specified by the manufacturer of the commercially available enzymes. After incubation with the restriction enzyme, protein is removed by extraction and the DNA recovered by precipitation. The cleaved fragments may be separated using, for example, polyacrylamide or agarose gel electrophoresis methods, according to methods known by those of skill in the art.

Sticky end cleavage fragments may be blunt ended using E. coli DNA polymerase 1 (Klenow) in the presence of the appropriate deoxynucleotide triphosphates (dNTPs) present in the mixture. Treatment with S1 nuclease also may be used, resulting in the hydrolysis of any single stranded DNA portions.

Ligations are performed using standard buffer and temperature conditions using T4 DNA ligase and ATP. Alternatively, restriction enzyme digestion of unwanted fragments can be used to prevent ligation.

Standard vector constructions can include specific antibiotic resistance elements. Ligation mixtures are transformed into a suitable host, and successful transformants selected by antibiotic resistance or other markers. Plasmids from the transformants can then be prepared according to methods known to those in the art usually following a chloramphenicol amplification as reported by Clewell et al. (J. Bacteriol. 110:667, 1972) may be added. The DNA is isolated and analyzed, usually by restriction enzyme analysis and/or sequencing. Sequencing may be by the well-known dideoxy method of Sanger et al. (Proc. Natl. Acad. Sci. USA 74:5463, 1977) as further described by Messing et al. (Nucleic Acid Res. 2:309, 1981), or by the method reported by Maxam et al. (Meth. Enzymol. 65:499, 1980). Problems with band compression, which are sometimes observed in GC rich regions, can be overcome by use of, for example, T-deazoguanosine or inosine, according to the method reported by Barr et al. (Biotechniques 4:428, 1986).

Host cells can be genetically modified with the vectors of the present invention, which may be a cloning vector or an expression vector. The vector may be in the form of a plasmid, a viral particle, a phage, etc. The genetically modified host cells can be cultured in conventional nutrient media modified as appropriate for activating promoters, selecting transformants/transfectants or amplifying the subunit-encoding polynucleotide. The culture conditions, such as temperature, pH and the like, generally are similar to those previously used with the host cell selected for expression, and will be apparent to those of skill in the art.

Both prokaryotic and eukaryotic host cells may be used for expression of desired coding sequences when appropriate control sequences (e.g., promoter sequences) that are compatible with the designated host are used. For example, among prokaryotic hosts, Escherichia coli is frequently used. Also, for example, expression control sequences for prokaryotes include but are not limited to promoters, optionally containing operator portions, and ribosome binding sites. Transfer vectors compatible with prokaryotic hosts can be derived from, for example, the plasmid pBR322 that contains operons conferring ampicillin and tetracycline resistance, and the various pUC vectors, that also contain sequences conferring antibiotic resistance markers. These markers may be used to obtain successful transformants by selection. Commonly used prokaryotic control sequences include but are not limited to the lactose operon system (Chang et al. Nature 198:1056, 1977), the tryptophan operon system (reported by Goeddel et al. (Nucleic Acid Res. 8:4057, 1980) and the lambda-derived P1 promoter and N gene ribosome binding site (Shimatake et al. Nature 292:128, 1981), the hybrid Tac promoter (De Boer et al. Proc. Natl. Acad. Sci. USA 292:128, 1983) derived from sequences of the trp and lac UV5 promoters. The foregoing systems are particularly compatible with E. coli; however, other prokaryotic hosts such as strains of Bacillus or Pseudomonas may be used if desired.

Eukaryotic hosts include yeast and mammalian cells in culture systems. Pichia pastoris, Saccharomyces cerevisiae and S. carlsbergensis are commonly used yeast hosts. Yeast-compatible vectors carry markers that permit selection of successful transformants by conferring protrophy to auxotrophic mutants or resistance to heavy metals on wild-type strains. Yeast compatible vectors may employ the 2-μ origin of replication (Broach et al. Meth. Enzymol. 101:307, 1983), the combination of CEN3 and ARS1 or other means for assuring replication, such as sequences that will result in incorporation of an appropriate fragment into the host cell genome. Control sequences for yeast vectors are known in the art and include but are not limited to promoters for the synthesis of glycolytic enzymes, including the promoter for 3-phosphoglycerate kinase. (See, for example, Hess et al. J. Adv. Enzyme Reg. 7:149, 1968; Holland et al. Biochemistry 17:4900, 1978; and Hitzeman J. Biol. Chem. 255:2073, 1980). For example, some useful control systems are those that comprise the glyceraldehyde-3-phosphate dehydrogenase (GAPDH) promoter or alcohol dehydrogenase (ADH) regulatable promoter, terminators also derived from GAPDH, and, if secretion is desired, leader sequences from yeast alpha factor. In addition, the transcriptional regulatory region and the transcriptional initiation region which are operably linked may be such that they are not naturally associated in the wild-type organism.

Host cells useful for expression of the polynucleotides of the present invention may be primary cells or cells of cell lines. Mammalian cell lines available as hosts for expression are known in the art and are available from depositories such as the American Type Culture Collection. These include but are not limited to HeLa cells, human embryonic kidney (HEK) cells, Chinese hamster ovary (CHO) cells, baby hamster kidney (BHK) cells, GH4Cl cells (a clonal strain of rat pituitary tumor cells) and others. Suitable promoters for mammalian cells also are known in the art and include viral promoters such as that from Simian Virus 40 (SV40), Rous sarcoma virus (RSV), adenovirus (ADV), bovine papilloma virus (BPV) and cytomegalovirus (CMV). Mammalian cells also may require terminator sequences and poly A addition sequences; enhancer sequences which increase expression also may be included, and sequences which cause amplification of the gene also may be desirable. These sequences are known in the art. Vectors suitable for replication in mammalian cells may include viral replicons, or sequences which ensure integration of the appropriate sequences encoding the T6′S variant TM2 domain, the T6′S variant α7 subunit, and/or the T6′S variant nACh receptor into the host genome. An example of such a mammalian expression system is described in Gopalakrishnan et al. Eur. J. Pharmacol.-Mol. Pharmacol. 290: 237-246, 1995).

Other eukaryotic systems available as hosts for expression are also known, as are methods for introducing polynucleotides into such systems, such as amphibian cells using methods described in Briggs et al. (Neuropharmacol. 34:583-590, 1995), insect cells using methods described in Summers and Smith, Texas Agricultural Experiment Station Bulletin No. 1555 (1987), and the like.

The baculovirus expression system can be used to generate high levels of recombinant proteins in insect host cells. The baculovirus expression system allows for high level of protein expression, while post-translationally processing the protein in a manner similar to mammalian cells. These expression systems use viral promoters that are activated following baculovirus infection to drive expression of cloned genes in the insect cells (O'Reilly et al. (1992) Baculovirus Expression Vectors: A Laboratory Manual, In/Oxford University Press).

Transfection may be by any known method for introducing polynucleotides into a host cell, including packaging the polynucleotide in a virus and transducing a host cell with the virus, by direct uptake of the polynucleotide by the host cell, and the like, which methods are known to those skilled in the art. The transfection procedures selected depend upon the host to be transfected.

The expression of the polynucleotide encoding the T6′S variant TM2 domain, the T6′S variant α7 subunit, and/or the T6′S variant nACh receptor may be detected by use of a radioligand selective for the receptor. For example, for the nicotinic cholinergic receptor, such a ligand may be [¹²⁵¹]α-bungarotoxin. However, any radioligand binding technique known in the art may be used to detect the receptor subunit (see, e.g., Winzor et al. (1995) Quantitative Characterization of Ligand Binding, Wiley-Liss, Inc., N.Y.). Alternatively, expression can be detected by utilizing antibodies or functional measurements which are well known to those skilled in the art.

The T6′S variant TM2 domain polypeptide, the T6′S variant α7 subunit polypeptide, or the T6′S variant nACh receptor polypeptide can be recovered and purified from genetically modified host cell cultures expressing the same by known methods including ammonium sulfate or ethanol precipitation, acid extraction, anion or cation exchange chromatography, phosphocellulose chromatography, hydrophobic interaction chromatography, hydroxyapatite chromatography or lectin chromatography. Protein refolding steps can be used, as necessary, in completing configuration of the protein. Finally, high performance liquid chromatography (HPLC) can be employed for final purification steps.

The T6′S variant TM2 domain polypeptide, the T6′S variant α7 subunit polypeptide, or the T6′S variant nACh receptor polypeptide, or functional fragments or homologues thereof, of the present invention, also may be synthesized by conventional techniques known in the art, for example, by chemical synthesis such as solid phase peptide synthesis. In general, these methods employ either solid or solution phase synthesis methods (See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis).

In one preferred system, either the DNA or the RNA derived therefrom, both of which encode the desired T6′S variant TM2 domain polypeptide, T6′S variant α7 subunit polypeptide, and/or T6′S variant nACh receptor polypeptide, may be expressed by direct injection into a cell, such as a Xenopus laevis oocyte. Using this method, the functionality of the polypeptide encoded by the DNA or the mRNA can be evaluated as follows (see Dascal CRC Crit. Rev. Biochem. 22:317-387, 1987). A T6′S variant-encoding polynucleotide is injected into an oocyte for translation into a functional receptor subunit. The function of the expressed T6′S variant TM2 domain polypeptide, T6′S variant α7 subunit polypeptide, and/or T6′S variant nAChR polypeptide can be assessed in the oocyte by a variety of electrophysiological techniques including intracellular voltage recording, two-electrode voltage clamp, patch clamp methods, and the like. The cation-conducting channel intrinsic to the nAChR opens in response to ACh or other nicotinic cholinergic agonists, permitting the flow of transmembrane current. This current can be monitored directly by voltage clamp techniques or indirectly by intracellular voltage recording, wherein changes in membrane potential due to the induced current are measured. Alternatives can include measurement of ion flux or fluorescent probes sensitive to transmembrane potential or changes in ion activity.

Receptors expressed in a genetically modified host cell may be used to identify compounds that modulate nAChR activity. In this regard, the specificity of the binding of a compound showing affinity for the receptor is demonstrated by measuring the affinity of the compound for cells expressing the receptor or membranes from these cells. This may be done by measuring specific binding of labeled (e.g., radioactive) compound to the cells, cell membranes or isolated receptor, or by measuring the ability of the compound to displace the specific binding of a standard labeled ligand. Expression of variant receptors and screening for compounds that bind to, or inhibit the binding of labeled ligand to these cells or membranes provides a method for rapid selection of compounds with high affinity for the receptor. These compounds may be agonists, antagonists or modulators of the receptor.

Expressed receptors also may be used to screen for agents, such as chemical compounds, that modulate nicotinic acetylcholine receptor activity. One method for identifying agents that modulate nAChR activity comprises providing a cell that expresses a polynucleotide encoding a T6′S variant (e.g., T6′S variant TM2 domain polypeptide, a T6′S variant α7 subunit polypeptide, and/or a T6′S variant nACh receptor polypeptide of the present invention), contacting a test agent with the cell and measuring the effect of the test compound on the variant receptor activity. The cell may be a bacterial cell, a mammalian cell, a yeast cell, an amphibian cell or any other cell expressing the receptor. Preferably, the cell is a mammalian cell or an amphibian cell. Thus, for example, a test agent is evaluated for its ability to elicit an appropriate response, e.g., the stimulation of transmembrane current flow, for its ability to inhibit the response to a cholinergic agonist, or for its ability to modulate the response to an agonist or antagonist. The contacting step in the methods of the invention can involve combining or mixing the test agent and the cell, for example.

In one embodiment, the present invention provides a method for identifying agents that modulate nicotinic acetylcholine receptor (nAChR) activity, comprising: (a) providing a host cell that expresses a polynucleotide encoding a variant α7 nAChR polypeptide having a TM2 domain, wherein the variant α7 nAChR polypeptide has a serine residue in the sixth amino acid position of the TM2 domain; (b) contacting a test agent with the host cell; and (c) determining: (i) the effect of the test agent on the variant α7 nAChR polypeptide or the host cell expressing the polynucleotide, or (ii) the binding of the test agent to the host cell or the variant α7 nAChR polypeptide, or (iii) both (c) (i) and (c) (ii).

In addition, expressed receptors may be used to screen agents, such as chemical compounds, that exhibit either a cytoprotective or cytotoxic effect. Abnormal activation of membrane channels is a potential cause of neurological disorders, such as neurodegenerative diseases (See, for example, Salamone, F. et al., MJM, 2000, 5:90-97; Cooper, E. C. and Jan, L. Y. Proc. Natl. Acad. Sci. USA, 1999, 96:4759-4766; Sharples, C. and Wonnacott, S. Neuronal Nicotinic Receptors, October 2001, 19:1-12; Mihailescu, S. and Drucker-Colin, R. Arch. Med. Res., 2000, 31:131-144; Papke, R. L. et al., Euro. J. Pharm., 2000, 393:179-195; Newhouse, P. A. and Kelton, M. Pharm. Acta Helv., 2000, 74:91-101; Newhouse, P. A. et al., Clin. Pharm., 1997, 11:206-228; Lloyd, G. K. and Williams, M. J. Pharm. Exp. Therapies, 2000, 292:461-467; Hollady, M. W. et al., J. Med. Chem., 1997, 40:4169-4194; Benowitz, N. L. Annu. Rev. Pharm. Toxicol., 1996, 36:597-613; Freedman, R. et al., Harvard Rev. Psych., 1994, 2:179-192; and Freedman, R. et al., J. Chem. Neuroanatomy, 2000, 20:299-306). Examples of such neurological disorders include, but are not limited to, presenile dementia (early onset Alzheimer's disease); senile dementia (dementia of the Alzheimer's type); movement disorders such as Parkinsonism including Parkinson's disease, Huntington's chorea, tardive dyskinesia, hyperkinesias, tremor, epilepsy, Tourette syndrome or other tic disorders; mania; attention deficit disorder; attention deficit hyperactivity disorder; sleep-wake disorder; chronic-fatigue syndrome; neuropathic pain; addiction (e.g., nicotine/smoking addiction); anxiety; dyslexia; schizophrenia; and obsessive-compulsive disorder.

The T6′S variant of the present invention can be used to screen for agents useful in treating disorders caused by alterations in sensory gating, immunofunction and neuropathic pain (e.g., pain associated with cancerous conditions, post herpatic neuralgia, diabetic neuropathy and osteoarthritis), as well as the foregoing list of neurological disorders.

Accordingly, nicotinic drugs are considered potential therapeutic agents in several neurological disorders, including the foregoing list of neurological disorders. Compounds that activate nAChRs, especially of the α7 subtype, have been found to have in vivo activity in models of cognition enhancement (U.S. Pat. No. 5,741,802, issued Apr. 21, 1998). Activation of the wild-type α7 nAChR appears to elicit cytoprotective properties (e.g., reduced cell lysis, see Donnelly-Roberts et al. Brain Res., 1996, 719:36-44). Although it is not yet conclusively established whether a full agonist or partial agonist is preferable, nor, if the latter, what type of partial agonist is best (e.g., one that stabilizes the open and desensitized states or one that stabilizes the open and resting states of the receptor), the T6′S variant of the present invention can be used to evaluate these questions, and to select among ligands for specific types of partial agonists or specific types of antagonists.

Thus, α7 nAChR ligand pharmacology can be studied through the use of the T6′S variant of the present invention (e.g., T6′S variant TM2 domain, T6′S variant α7 subunit, and/or T6′S variant nAChR). The ability of a ligand to stabilize the desensitized state of the receptor can be evaluated by comparing the ligand's potency and efficacy at the T6′S variant nAChR to its potency and efficacy at the wild-type α7 nAChR, for example. Alternatively, the ligand's interaction with the T6′S variant nAChR can be compared to other variants of the wild-type α7 nAChR. The interaction of agents with variants and the wild-type nAChR can be identified using several methods, including, but not limited to, electrophysiologic measurement of transmembrane current flow or electrical potential, measurement of the fluorescence of potential-sensitive or ion-sensitive dyes, or measurement of radioactive ion flux and a variety of α7 nAChR expression systems, for example, transfected mammalian cells in culture or injected amphibian cells. In preferred embodiments, the responses are measured with a high throughput electrophysiology system and/or rapid fluorescence assays such as the FlipR (fluorometric imaging plate reader) system.

In addition to screening test agents, the expressed T6′S variant (e.g., T6′S variant TM2 domain, T6′S variant α7 subunit, and/or T6′S variant nACh receptor) may be used to investigate mechanisms of cytotoxicity and cytoprotection. The evidence that activation of α7 nAChR is cytoprotective comes from the finding that nAChR agonists elicit cytoprotection in cells expressing the wild-type α7 nAChR subunit and that this cytoprotection is inhibited by selective α7 antagonists (for example, see Donnelly-Roberts et al. Brain Res., 719:36-44, 1996).

Cytoprotective or cytotoxic agents that interact with the T6′S variant (e.g., T6′S variant TM2 domain, T6′S variant α7 subunit, and/or T6′S variant nACh receptor) of the present invention may be identified using several methods. One such method comprises providing a cell that expresses the T6′S variant TM2 domain, T6′S variant α7 subunit, and/or T6′S variant nACh receptor of the present invention, combining a test agent with the cell, and monitoring the cell for an indicator of cytotoxicity. If it is necessary to control spontaneous action of the variant nAChR subunit, it may be stably expressed in a recombinant mammalian cell line under the control of an inducible promoter, e.g., the LacSwitch system which is inducible by isopropylthiogalactoside (“IPTG”). Expression of the T6′S variant TM2 domain, T6′S variant α7 subunit, and/or T6′S variant nACh receptor would be maintained at a low level until induction by the addition of IPTG. Alternatively, with or without an inducible promoter, the transfected cells could be cultured in the presence of an α7 blocker, such as methyllycaconitine (“MLA”) or mecamylamine, that would prevent or reduce cytotoxic action. Both blockers are reversible, permitting one to measure the effect of the test agent on α7 nAChR function after the blocker is removed (e.g., washed out).

Cytoprotective agents can be identified by their ability to reduce cell death while cytotoxic agents can be identified by their ability to promote cell death. That these effects are mediated by the α7 subunit, T6′S variant or wild-type, can be identified by the ability of an α7 blocker to prevent the effect. Cell death, or cytotoxicity, can be monitored by a variety of techniques including but not limited to measurement of cell number or density in the culture, of cell growth rate (e.g., incorporation of labeled nucleotide or amino acid), or of cell integrity for example by uptake of a dye (e.g., trypan blue is excluded by healthy cells, or by inclusion of MTT by healthy cells), or by the release of a cytoplasmic constituent such as lactate dehydrogenase (LDH). Cytoprotective agents may also be screened for their ability to antagonize a variant nAChR to a greater extent than a wild-type nAChR, or for their ability to augment the decay rate of variant nAChR compared to the wild-type nAChR.

In one embodiment, the subject invention provides a method for identifying a cytoprotective agent, comprising: (a) providing a host cell that expresses a polynucleotide encoding a variant α7 nAChR polypeptide having a TM2 domain, wherein the variant α7 nAChR polypeptide has a serine residue in the sixth amino acid position of the TM2 domain (such as a T6′S variant α7 subunit comprising SEQ ID NO:2); contacting a test agent (such as a putative wild-type α7 receptor agonist) with the host cell; and (c) monitoring the host cell or cellular function for an indication of cytotoxicity. In a specific embodiment, whether the test agent overstimulates the variant α7 nAChR polypeptide is determined. Because the variant α7 nAChR polypeptide exhibits amplified pharmacologic responses compared to the wild-type α7 receptor, determination of overstimulation of the variant α7 nAChR polypeptide can indicate or connote that the test agent is sufficient to activate the wild-type α7 receptor at a cytoprotective level. In this way, a potential therapeutic agent may elicit a cytotoxic response in the variant α7 nAChR polypeptide of the present invention but elicit a cytoprotective response in the wild-type α7 receptor.

It has been determined that native and degenerate peptides derived from the α7 nicotinic receptor (such as the human α7 receptor) are useful as ligands for β amyloid peptides. The neurotoxic β amyloid₁₋₄₂ (Aβ₁₋₄₂) is abundantly present in the amyloid plaques of Alzheimer's disease (AD) brains and also modulates cholinergic functions which are critical in memory and cognitive neurophysiology (Auld D. et al., Trends Neurosci 21:43-49, 1998). Aβ₁₋₄₂ interacts selectively with high affinity to the neuronal α7 nicotinic receptor. U.S. Pat. No. 6,441,049 (Reitz et al.; issued Aug. 27, 2002) describes methods of treating a neurodegenerative disorder in a subject by administering a compound that inhibits the interaction of Aβ with the α7 receptor. U.S. Patent publication U.S. 2003/0092613 (Lee et al.; filed Aug. 13, 2001) describes methods for diagnosing Alzheimer's disease involving the binding of α7 that allows the measurement of Aβ peptides in biological samples, methods for designing a drug discovery assay for identifying α7 nAChR modulators based on the interaction of α7 with Aβ (the disclosure of which is expressly incorporated herein by reference in its entirety). The subject invention encompasses these methods, wherein polypeptides comprising the T6′S domain of the present invention, and which interact with Aβ, are substituted for the α7 and variant peptides described in U.S. Patent publication U.S. 2003/0092613.

The present invention also includes isolated polynucleotides (e.g., detection probes or primers) capable of selectively hybridizing to a nucleic acid sequence encoding a T6′S variant TM2 domain, T6′S variant α7 subunit, and/or T6′S variant nACh receptor. For example, the isolated polynucleotide can be capable of selectively hybridizing to a nucleic acid sequence comprising SEQ ID NO:18, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a functional fragment or homologue of any of the foregoing. Such a detection probe will comprise a contiguous/consecutive span of at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 nucleotides. Labeled probes or primers are labeled with a radioactive compound or with another type of label as set forth above (e.g., 1) radioactive labels, 2) enzyme labels, 3) chemiluminescent labels, 4) fluorescent labels, or 5) magnetic labels). Alternatively, non-labeled nucleotide sequences may be used directly as probes or primers; however, the sequences are generally labeled with a radioactive element (³²P, ³⁵S, ³H, ¹²⁵I) or with a molecule such as biotin, acetylaminofluorene, digoxigenin, 5-bromo-deoxyuridine, or fluorescein to provide probes that can be used in numerous applications. Preferably, the isolated polynucleotide specifically hybridizes with at least 8, 9, 10, 11, 12, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 nucleotides of the nucleotide sequence encoding the T6′S variant α7 subunit. More preferably, the isolated polynucleotide specifically hybridizes with at least 4, 5, 6, 7, 8, 9, 10, 11, 12, 15, 16, 17, 18, 19, 20, 21, 22, 23, 24, 25, 26, 27, 28, 29, 30, 35, 40, 45, 50, 55, or 60 nucleotides of the T6′S variant TM2 domain (e.g., of SEQ ID NOs: 18, 21, 22, 23, 24, 25, 26, 27, 28, 29, or 30). Optionally, the isolated polynucleotide can specifically hybridize with nucleotides within that portion of the target sequence which encodes the T6′ S variant TM2 domain and outside that which encodes the TM2 domain. Optionally, hybridization outside the T6′S variant TM2 domain can include hybridization to non-coding nucleotides. Such polynucleotides can be produced by recombinant or synthetic techniques.

Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under low, intermediate, or high stringency conditions by techniques well known in the art, as described, for example, in Keller, G. H., M. M. Manak, 1987, DNA Probes, Stockton Press, New York, N.Y., pp. 169-170.

For example, hybridization of immobilized DNA on Southern blots with ³²P-labeled gene-specific probes can be performed by standard methods (Maniatis et al. [1982] Molecular Cloning: A Laboratory Manual, Cold Spring Harbor Laboratory, New York). In general, hybridization and subsequent washes can be carried out under intermediate to high stringency conditions that allow for detection of target sequences with homology to the exemplified polynucleotide sequence. For double-stranded DNA gene probes, hybridization can be carried out overnight at 20-25° C. below the melting temperature (Tₘ) of the DNA hybrid in 6×SSPE, 5× Denhardt's solution, 0.1% SDS, 0.1 mg/ml denatured DNA. The melting temperature is described by the following formula (Beltz et al. [1983] Methods of Enzymology, R. Wu, L. Grossman and K. Moldave [eds.] Academic Press, New York 100:266-285).

Tₘ=81.5° C.+16.6 Log[Na⁺]+0.41(% G+C)−0.61(% formamide)−600/length of duplex in base pairs.

Washes are typically carried out as follows:
(1) twice at room temperature for 15 minutes in 1×SSPE, 0.1% SDS (low stringency wash);
(2) once at Tₘ-20° C. for 15 minutes in 0.2×SSPE, 0.1% SDS (intermediate stringency wash).

For oligonucleotide probes, hybridization can be carried out overnight at 10-20° C. below the melting temperature (Tₘ) of the hybrid in 6×SSPE, 5× Denhardt's solution, 0.1% SDS, 0.1 mg/ml denatured DNA. Tₘ for oligonucleotide probes can be determined by the following formula:

Tₘ(° C.)=2(number T/A base pairs)⁺4(number G/C base pairs) (Suggs et al. [1981] ICN-UCLA Symp. Dev. Biol. Using Purified Genes, D. D. Brown [ed.], Academic Press, New York, 23:683-693).

Washes can be carried out as follows:
(1) twice at room temperature for 15 minutes 1×SSPE, 0.1% SDS (low stringency wash);
2) once at the hybridization temperature for 15 minutes in 1×SSPE, 0.1% SDS (intermediate stringency wash).

In general, salt and/or temperature can be altered to change stringency. With a labeled DNA fragment>70 or so bases in length, the following conditions can be used:
Low: 1 or 2×SSPE, room temperature
Low: 1 or 2×SSPE, 42° C.
Intermediate: 0.2× or 1×SSPE, 65° C.
High: 0.1×SSPE, 65° C.

By way of another non-limiting example, procedures using conditions of high stringency can also be performed as follows: Pre-hybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C., the preferred hybridization temperature, in pre-hybridization mixture containing 100 μg/ml denatured salmon sperm DNA and 5-20×10⁶ cpm of ³²P-labeled probe. Alternatively, the hybridization step can be performed at 65° C. in the presence of SSC buffer, 1×SSC corresponding to 0.15M NaCl and 0.05 M Na citrate. Subsequently, filter washes can be done at 37° C. for 1 h in a solution containing 2×SSC, 0.01% PVP, 0.01% Ficoll, and 0.01% BSA, followed by a wash in 0.1×SSC at 50° C. for 45 min. Alternatively, filter washes can be performed in a solution containing 2×SSC and 0.1% SDS, or 0.5×SSC and 0.1% SDS, or 0.1×SSC and 0.1% SDS at 68° C. for 15 minute intervals. Following the wash steps, the hybridized probes are detectable by autoradiography. Other conditions of high stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Another non-limiting example of procedures using conditions of intermediate stringency are as follows: Filters containing DNA are pre-hybridized, and then hybridized at a temperature of 60° C. in the presence of a 5×SSC buffer and labeled probe. Subsequently, filters washes are performed in a solution containing 2×SSC at 50° C. and the hybridized probes are detectable by autoradiography. Other conditions of intermediate stringency which may be used are well known in the art and as cited in Sambrook et al., 1989, Molecular Cloning, A Laboratory Manual, Second Edition, Cold Spring Harbor Press, N.Y., pp. 9.47-9.57; and Ausubel et al., 1989, Current Protocols in Molecular Biology, Green Publishing Associates and Wiley Interscience, N.Y. are incorporated herein in their entirety.

Duplex formation and stability depend on substantial complementarity between the two strands of a hybrid and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions and deletions can be produced in a given polynucleotide sequence in many ways, and these methods are known to an ordinarily skilled artisan. Other methods may become known in the future.

A “complementary” polynucleotide sequence, as used herein, generally refers to a sequence arising from the hydrogen bonding between a particular purine and a particular pyrimidine in double-stranded nucleic acid molecules (DNA-DNA, DNA-RNA, or RNA-RNA). The major specific pairings are guanine with cytosine and adenine with thymine or uracil. A “complementary” polynucleotide sequence may also be referred to as an “antisense” polynucleotide sequence or an “antisense sequence”.

The present invention also includes pharmaceutical compositions useful for treating conditions associated with neurodegenerative processes, enzymatic function, affective disorders or immuno function, comprising a pharmaceutically acceptable carrier and an agent that regulates the function of a T6′S variant TM2 domain comprising the amino acid sequence of SEQ ID NO:2, the function of the T6′S variant α7 subunit comprising the amino acid sequence of SEQ ID NO:2, or SEQ ID NO:20, or amino acids 23 to 502 of SEQ ID NO:20, and/or the function of the T6′S variant nACh receptor comprising the amino acid sequence of SEQ ID NO:2, or SEQ ID NO:20, or amino acids 23 to 502 of SEQ ID NO:20; or a functional fragment or homologue of any of the foregoing. Such homologues include, for example, the mature peptides of SEQ ID NOs. 7, 9, 11, 13, 15, and 17, wherein the threonine in the sixth amino acid position of the TM2 domain has been substituted with a serine residue.

The present invention also includes pharmaceutical compositions useful for treating conditions associated with neurodegenerative processes, enzymatic function, affective disorders or immuno function, comprising a pharmaceutically acceptable carrier and a polynucleotide encoding the T6′S variant TM2 domain comprising the amino acid sequence of SEQ ID NO:2, the T6′S variant α7 subunit comprising the amino acid sequence of SEQ ID NO:2, or SEQ ID NO:20, or amino acids 23 to 502 of SEQ ID NO:20, and/or the T6′S variant nACh receptor comprising the amino acid sequence of SEQ ID NO:2, or SEQ ID NO:20, or amino acids 23 to 502 of SEQ ID NO:20; or a functional fragment or homologue of any of the foregoing. The polynucleotide is preferably a component of a host cell or an expression vector, which further comprises a control sequence that directs expression of the polynucleotide, whereby the polynucleotide is expressed within the cell upon transplantation in vivo. In other embodiments, the polynucleotide T6′S variant TM2 domain can encode homologs of SEQ ID NO:20. Such homologues include, for example, the mature peptides of SEQ ID NOs. 7, 9, 11, 13, 15, and 17, wherein the threonine in the sixth amino acid position of the TM2 domain has been substituted with a serine residue.

The present further includes methods for treating one or more of the aforementioned conditions by administering an agent that regulates the function of a T6′S variant of the present invention, or by administering a T6′S variant polypeptide of the present invention, or by administering a polynucleotide encoding a T6′S variant polypeptide of the present invention, to a patient in need thereof. Preferably, the agent, T6′S polypeptide, or T6′S polynucleotide is administered a pharmaceutical composition of the subject invention. The pharmaceutical compositions of the present invention can be administered to a patient by any route that results in the desired therapeutic effect. For example, the pharmaceutical compositions can be administered intravenously (I.V.), intramuscularly (I.M.), subcutaneously (S.C.), intradermally (I.D.), orally, intranasally, etc.

Thus, the present invention includes methods of gene therapy whereby polynucleotides encoding the T6′S variant of the present invention is delivered to a patient, and the T6′S variant-encoding polynucleotide is expressed in vivo. The term “gene therapy”, as used herein, refers to the transfer of genetic material (e.g., DNA or RNA) of interest into a host to treat or prevent a genetic or acquired disease or condition phenotype. The genetic material of interest encodes a product (e.g., a protein, polypeptide, peptide, or functional RNA) whose production in vivo is desired. For example, the genetic material of interest can encode a hormone, receptor, enzyme, polypeptide or peptide of therapeutic value. For a review see, in general, the text “Gene Therapy” (Advances in Pharmacology 40, Academic Press, 1997).

Two basic approaches to gene therapy have evolved: (1) ex vivo and (2) in vivo gene therapy. The methods of the subject invention encompass either or both. In ex vivo gene therapy, cells are removed from a patient and, while being cultured, are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to produce the transfected gene product in situ.

In in vivo gene therapy, target cells are not removed from the subject, rather the gene to be transferred is introduced into the cells of the recipient organism in situ, that is within the recipient. Alternatively, if the host gene is defective, the gene is repaired in situ. These genetically altered cells have been shown to produce the transfected gene product in situ.

The gene expression vector is capable of delivery/transfer of heterologous nucleic acid sequences into a host cell. The expression vector may include elements to control targeting, expression and transcription of the nucleic acid sequence in a cell selective manner as is known in the art. It should be noted that often the 5′UTR and/or 3′UTR of the gene may be replaced by the 5′UTR and/or 3′UTR of the expression vehicle.

The expression vector can include a promoter for controlling transcription of the heterologous material and can be either a constitutive or inducible promoter to allow selective transcription. The expression vector can also include a selection gene.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and include, for example, stable or transient transfection, lipofection, electroporation, and infection with recombinant viral vectors.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of a DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

The pharmaceutical compositions of the subject invention can be formulated according to known methods for preparing pharmaceutically useful compositions. Formulations are described in a number of sources which are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The present invention also includes antibodies (e.g., monoclonal or polyclonal) and antibody fragments that specifically bind to T6′S variant α7 TM2 domain comprising the amino acid sequence of SEQ ID NO: 2. Antibodies that are immunospecific for the polypeptides as set forth herein are specifically contemplated. In various embodiments, antibodies that do not cross-react with other proteins (such as A. marginale MSP5) are also specifically contemplated. Preferably, the antibody or antibody fragment does not specifically bind to the wild-type α7 TM2 domain. The antibodies of the subject invention can be prepared using standard materials and methods known in the art (see, for example, Monoclonal Antibodies: Principles and Practice, 1983; Monoclonal Hybridoma Antibodies: Techniques and Applications, 1982; Selected Methods in Cellular Immunology, 1980; Immunological Methods, Vol. II, 1981; Practical Immunology, and Kohler et al. [1975] Nature 256:495). These antibodies can further comprise one or more additional components, such as a solid support, a carrier or pharmaceutically acceptable excipient, or a label.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity, particularly neutralizing activity. “Antibody fragments” comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multi-specific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. [1975] Nature 256: 495, or may be made by recombinant DNA methods (see, e.g., U.S. Pat. No. 4,816,567). The “monoclonal antibodies” may also be isolated from phage antibody libraries using the techniques described in Clackson et al. [1991] Nature 352: 624-628 and Marks et al. [1991] J. Mol. Biol. 222: 581-597, for example.

The monoclonal antibodies described herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. [1984] Proc. Natl. Acad. Sci. USA 81: 6851-6855). Also included are humanized antibodies, such as those taught in U.S. Pat. No. 6,407,213 or 6,417,337 which are hereby incorporated by reference in their entirety.

“Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies [1994] Vol. 113:269-315, Rosenburg and Moore eds. Springer-Verlag, New York.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The terms “comprising”, “consisting of” and “consisting essentially of” are defined according to their standard meaning. The terms may be substituted for one another throughout the instant application in order to attach the specific meaning associated with each term. The phrases “isolated” or “biologically pure” refer to material that is substantially or essentially free from components which normally accompany the material as it is found in its native state. Thus, isolated peptides in accordance with the invention preferably do not contain materials normally associated with the peptides in their in situ environment. “Link” or “join” refers to any method known in the art for functionally connecting peptides, including, without limitation, recombinant fusion, covalent bonding, disulfide bonding, ionic bonding, hydrogen bonding, and electrostatic bonding.

As used in this specification and the appended claims, the singular forms “a”, an and “the” include plural reference unless the context clearly dictates otherwise. Thus, for example, a reference to “a receptor subunit” includes more than one such subunit, and the like.

The practice of the present invention can employ, unless otherwise indicated, conventional techniques of molecular biology, microbiology, recombinant DNA technology, electrophysiology, and pharmacology, that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover ed. 1985); Perbal, B., A Practical Guide to Molecular Cloning (1984); the series, Methods In Enzymology (S. Colowick and N. Kaplan eds., Academic Press, Inc.); Transcription and Translation (Hames et al. eds. 1984); Gene Transfer Vectors For Mammalian Cells (J. H. Miller et al. eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. eds. (1991) IRL Press)).

### Materials and Methods

cDNA clones. These experiments used the rat neuronal nAChR and the mouse muscle cDNA clones, which were obtained from Dr. Jim Boulter (UCLA).

Site-directed mutagenesis. Site-directed mutagenesis was performed using QuickChange™ kits (Strategene, LaJolla, Calif.). In brief, two complimentary oligonucleotides were synthesized which contained the desired mutation flanked by 10-15 bases of unmodified nucleotide sequence. Using a thermal cycler, Pfu DNA polymerase extends the sequence around the whole vector, generating a plasmid with staggered nicks. Each cycle builds only off the parent strands, and therefore there is no amplification of misincorporations. After 12-16 cycles, the product was treated with Dpn I, which digests the methylated parent DNA into numerous small pieces. The product was then transformed into E. coli cells, which repair the nicks.

Preparation of RNA. After linearization and purification of cloned cDNAs, RNA transcripts were prepared in vitro using the appropriate mMessage mMachine kit from Ambion Inc. (Austin, Tex.).

Expression in Xenopus oocvtes. Mature (>9 cm) female Xenopus laevis African toads (Nasco, Ft. Atkinson, Wis.) were used as a source of oocytes. Prior to surgery, frogs were anesthetized by placing the animal in a 2 g/L solution of MS222 (3-aminobenzoic acid ethyl ester). Oocytes were removed from an incision made in the abdomen.

In order to remove the follicular cell layer, harvested oocytes were treated with collagenase from Worthington Biochemical Corporation (Freehold, N.J.) for 2 hours at room temperature in calcium-free Barth's solution (88 mM NaCl, 10 mM HEPES pH 7.6, 0.33 mM MgSO₄, 0.1 mg/ml gentamicin sulfate). Subsequently, stage 5 oocytes were isolated and injected with 50 nl each of a mixture of the appropriate subunit cRNAs following harvest. Recordings were made 3 to 21 days after injection depending on the cRNAs being tested.

Voltage-clamp recording of whole oocate responses. Data were obtained by means of two-electrode voltage-clamp recording. Recordings were made at room temperature (21-24 deg. C.) in Frog Ringer's solution (115 mM NaCl, 10 mM HEPES, 2.5 mM KCl, and 1.8 mM CaCl₂, pH 7.3) with 1 μM atropine to inhibit muscarinic acetylcholine receptor responses. This extracellular solution was used for all experiments unless otherwise noted. Voltage electrodes were filled with 3M KCl, and current electrodes were filled with 250 mM CsCl, 250 mM CsF, and 100 mM EGTA (pH 7.3).

Bath solution and drug applications are applied through a linear perfusion system to oocytes placed in Lucite chamber with a total volume of 0.5 ml. Drug delivery involves pre-loading a 1.8 ml length of tubing at the terminus of the perfusion system, while a Mariotte flask filled with Ringer's solution is used to maintain constant perfusion. Applications of drug solutions are then synchronized with acquisition. Current responses are recorded using a PC interfaced to either a Warner OC-725C (Warner Instruments, Hamden, Conn.) or a GeneClamp 500 amplifier via a Digidata 1200 digitizer (Axon Instruments, Union City, Calif.). In addition, some oocyte recordings were made using a beta version of the OpusXpress 6000A (Axon Instruments, Union City, Calif.). OpusXpress is an integrated system that provides automated impalement and voltage clamp, which in our case permitted the study of four oocytes in parallel. Cells were automatically perfused with bath solution, and agonist solutions were delivered from a 96-well compound plate. In experiments using the OpusXpress system, the voltage and current electrodes were filled with 3 M KCl. In all experiments, bath flow rates were set at 6 ml/minute.

Experimental protocols and analysis of data from Xenopus oocytes. Current responses to drug application were studied under two-electrode voltage clamp at a holding potential of −50 mV unless otherwise noted (−60 mV for the OpusXpress system). Holding currents immediately prior to agonist application were subtracted from measurements of the peak response to agonist. All drug applications were separated by a wash period of 5 minutes unless otherwise noted. At the start of recording, all oocytes received two initial control applications of ACh. Subsequent drug applications were normalized to the second ACh application in order to control for the level of channel expression in each oocyte. Means and standard errors (SEM) were calculated from the normalized responses of at least four oocytes for each experimental concentration.

For concentration-response relations, data were plotted using Kaleidagraph 3.0.2 (Abelbeck Software; Reading, Pa.), and curves were generated using the Hill equation:

$$Response = \frac{{{I}_{max} [ agonist ]}^{n}}{{[ agonist ]}^{n} + {( EC50 )}^{n}}$$

where Iₘₐₓ denotes the maximal response for a particular agonist/subunit combination, and n represents the Hill coefficient. Iₘₐₓ, n, and the EC₅₀ were all unconstrained for the fitting procedures.

For experiments measuring barium permeability, oocytes were perfused with barium Ringers (low barium: 90.7 mM NaCl, 2.5 mM KCl, 10 mM HEPES pH 7.3, 1.8 mM BaCl2, 48.6 mM sucrose; high barium: 90.7 mM NaCl, 2.5 mM KCl, 10 mM HEPES pH 7.3, 18 mM BaCl2). Shifts in reversal potential were measured by changing the holding potential from −40 mV to +30 mV by 10 mV increments. Calculations of barium-sodium permeability ratios using the extended GHK equation were performed using the Clampfit analysis portion of the pClamp software suite (Axon Instruments, Union City, Calif.). Barium was used instead of calcium to minimize the contribution of endogenous calcium-activated chloride channels (Sands, S. B. et al. Biophys. J., 1993, 65:2614-2621).

Calculations of peak amplitudes and net charge were made using pClamp either during acquisition or during subsequent Clampfit analysis. Note that measurement of net charge has been shown to be a more accurate indicator of fast α7 responses than measurement of peak response. An appropriate method using analysis of the area under the curve of agonist-evoked currents in oocytes has been previously published (Papke, R. L. and J. K. P. Papke Br. J. Pharmacol., 2002, 137:49-61). Baseline was defined for Clampfit statistics based on 20 s before drug application, the analysis region for peak and net charge analysis went from 5 s before the initiation of drug application and extended at least 135 s following. Area analysis data is provided for all receptor subtypes examined in this paper for comparison to wild-type α7.

Transfection and whole cell voltage clamp recording from GH4Cl cells. GH4Cl cells were cultured in F10 medium (Gibco, Carlsbad, Calif.) at 37° C., 5% CO₂. Cells were transiently transfected using Fugene (Roche, Indianapolis, Ind.), according to the manufacturer instructions. One microgram of WT or T6° F. mutant α7 cDNA (pTR-UF22, University of Florida, Gainesville, Fla.) was added to each 35-mm Petri dish, together with 0.5 or 1 μg of the cDNA encoding the red fluorescent DsRed protein (BD Biosciences Clontech, Palo Alto, Calif.). Cells were used 48-72 hours after transfection. Typical transfection efficiency was 10-25% using this method.

Whole-cell currents were recorded using an Axopatch 200B amplifier (Axon Instruments, Union City, Calif.) at room temperature. Cells were bathed in a solution containing 140 mM NaCl, 2.8 mM KCl, 2 mM CaCl₂, 2 mM MgCl₂, 10 mM glucose, 10 mM HEPES/NaOH (pH 7.3). Patch electrodes (tip resistances, 1-3 MΩ) were filled with 90 mM CsCl, 60 mM CsF, 10 mM HEPES/CsOH (pH 7.3). Cells were continuously superfused by a gravity-driven perfusion system connected to a PZ-150M piezoelectric switching device (EXFO Burleigh Products Group, Victor, N.Y.), with independent tubes for each solution placed ca. 100 μm from the cells. Currents were recorded at 2 KHz using pClamp 8 (Axon Instruments Union City, Calif.), at a membrane holding potential of −70 mV, unless otherwise indicated. Analysis was conducted using pClamp 9.

### EXAMPLE 1

### The T6′S Mutant Receptor Pharmacology Closely Resembles That of Wild-Type α7

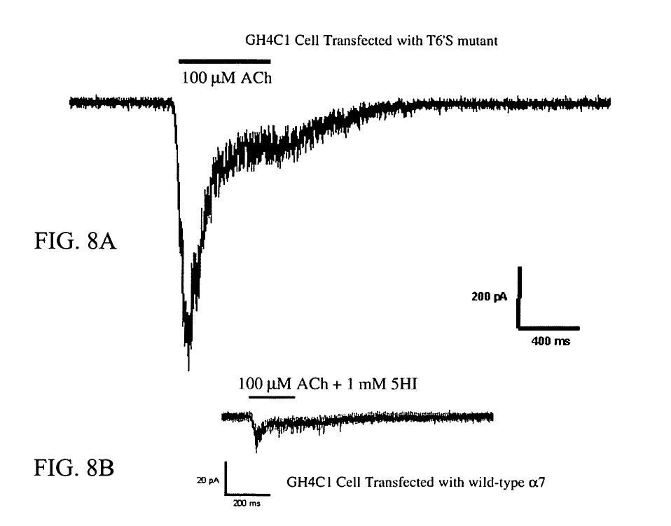

Raw data traces from transiently transfected GH4Cl cells show the larger amplitude and slower macroscopic kinetics of the T6′S mutant compared to wild-type α7 (FIGS. 8A and 8B), and that this observation carries over from oocyte studies to a mammalian cell under whole-cell voltage clamp. Table 2 shows the results of oocyte experiments examining representative concentrations of a battery of compounds, indicating the ability of the T6′S mutant to effectively emulate wild-type α7. For the drugs examined, the T6′S mutant is more like wild-type α7, particularly in regard to antagonists that have been converted to agonists in the L247T mutant.

FIGS. 8A and 8B and Table 2 show that the T6′S variant of the present invention yields much larger currents than wild-type α7 receptor, and provides a more faithful representation of the wild-type α7 pharmacology than does the commonly used L247T mutant.

TABLE 2

| | L247T | Wild type | T6'S |
|---|---|---|---|
| Choline EC50 | 12 μM | 300 μM | ≈360 μM |
| ACh EC₅₀ | 1 μm | 30 μM | 100 μM |
| GTS-21 (DMXB) | full | 32% agonist 5 μM | 12% agonist 3 μM |
| | agonist | | |
| AR-R17779 | ? | 78% agonist 10 μM | 90% agonist 30 μM |
| Cytisine | ? | 73% agonist 13 μM | 80% agonist 43 μM |
| Choline | ? | 100% agonist 300 μM | 95% agonist 2 mM |
| TC-2403 | ? | 16% agonist 240 μM | 18% agonist |
| | | | 400 μM |
| 4OH-GTS-21 | ? | 46% agonist 1.4 μM | 20% agonist 3.3 μM |
| Tropisetron | ? | 38% Agonist 0.3 μM | 30% 0.9 μM |
| Nor-Nicotine | ? | 50% Agonist 6 μM | 50% 64 μM |
| Bicuculline | agonist | antagonist | antagonist |
| 5-HT | agonist | antagonist | antagonist |
| Tubocurarine | agonist | antagonist | antagonist |
| Hexamethonium | agonist | antagonist | antagonist |
| Zinc | agonist | antagonist | antagonist |
| 5HI (5-hydroxy- | ? | potentiates | does not potentiate |
| indole) | | | |

### EXAMPLE 2

### Alpha7 TM2 6′ Mutants are Functionally Expressed in Oocvtes and Impact Absolute Current Amplitude

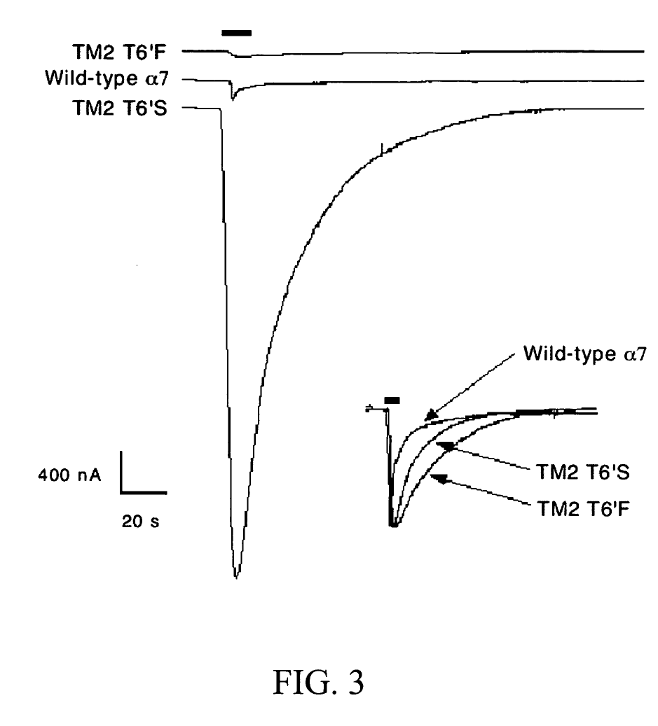

Raw data traces for each of the TM2 6′ single point mutations and wild-type α7 are shown in FIG. 3. A particularly obvious difference between the two 6′ mutant receptors and wild-type α7 is the consistently large peak current for the T6′S mutant and the relatively small peak for the T6° F. response. Quantification of differences in absolute amplitude is complicated by variations in the degree of receptor expression from cell to cell (which is why each cell is used as it's own control), however variations in current amplitude similar to those seen were regularly observed.

### EXAMPLE 3

### The α7 T6′F Mutation Increases ACh Potency While the T6′S Mutant Shows no Significant Change in ACh Potency Compared to Wild Type α7

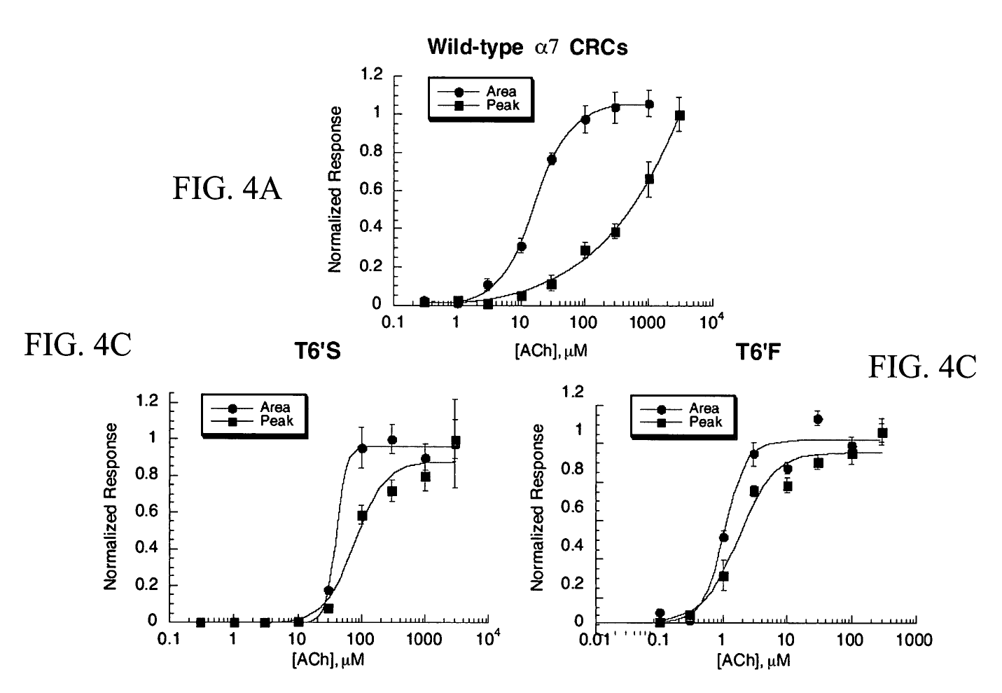

Concentration-response functions for wild-type α7 and each of the two TM2 6′ mutants are seen in FIGS. 4A-4C. Both net charge (area under the curve) and peak CRCs are shown to illustrate the differences in apparent ACh potency as a function of the analysis method used. Comparison of these analyses shows that the wild-type α7 receptor shows the greatest difference in apparent potency, while the two 6′ mutants show less difference between the two methods, probably due to their slower response kinetics (see methods). In addition, a dramatic increase in ACh potency is seen for the net charge analysis if the T6′F mutant, whereas the T6′S mutant is not significantly different from wild-type.

### EXAMPLE 4

### The α7 T6′F Mutation Abolishes Divalent Ion Permeability and Reduces Inward Current Rectification

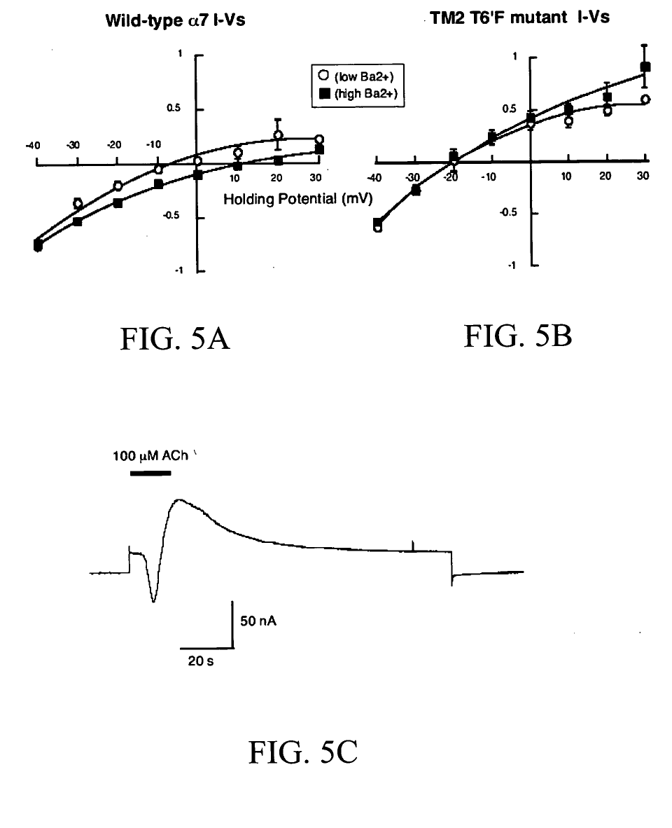

Current-voltage relationships in either high or low extracellular barium, show that the wild-type α7 receptor displays its characteristic inward rectification and permeability to divalent ions (FIGS. 5A and 5B). The extended GHK equation gives an estimated barium-to-sodium permeability ratio of approximately 4:1. By contrast, the T6′F mutant receptor shows little current rectification, particularly under conditions of high extracellular barium and there was a complete lack of a shift in reversal potential with varying extracellular divalent ion concentrations. The GHK equation applied to these results gives a barium to sodium permeability ratio that is near zero. Measurements of barium ion permeability in the T6′S mutant were complicated by biphasic currents like the example shown FIG. 5C. These biphasic currents occurred near the reversal potential for chloride and possibly reflect an interaction between barium and the calcium gated chloride channels that are constitutively expressed by Xenopus oocytes. Given the slow, large amplitude macroscopic responses of the α7 T6′S mutant, it is possible that low potency interactions between barium and these calcium gated chloride channels would be significantly enhanced.

### EXAMPLE 5

### Succinylcholine is a Selective Agonist of Muscle-Type Receptors, and is Also an Agonist of α7 T6′F Mutant Receptors, But Not the T6′S Mutant

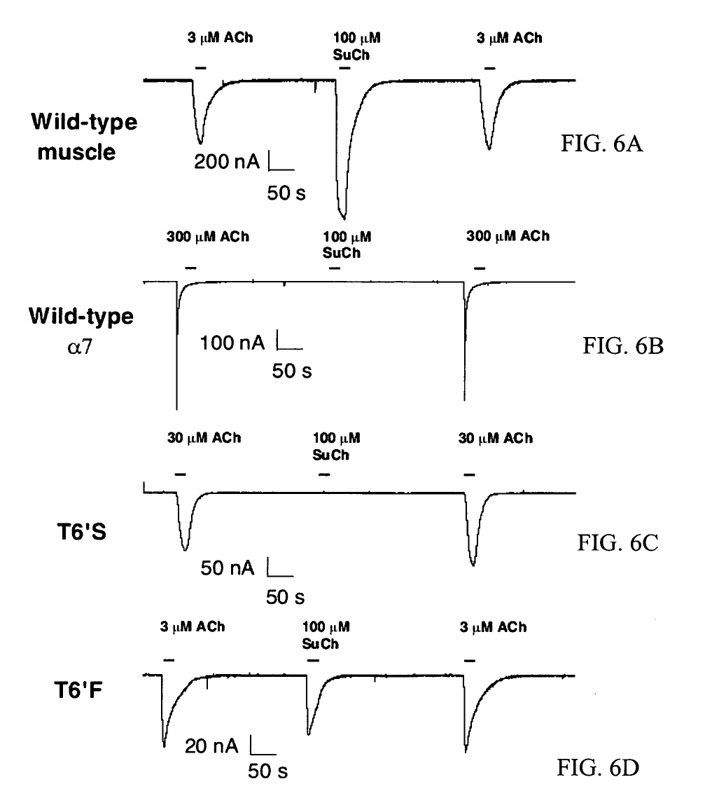

The comparative effect of 100 μM succinylcholine on muscle or wild-type α7 receptors, shows that succinylcholine is relatively ineffective in activating wild-type α7 (FIGS. 6A and 6B). A dramatic increase in sensitivity to succinylcholine can be seen in the T6′F mutant receptor, while the T6′S mutant shows no virtually no response at this concentration.

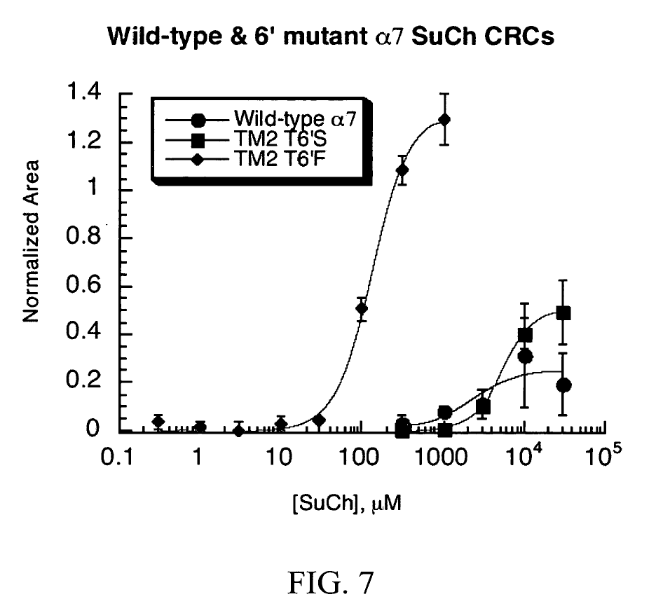

The concentration-response functions for succinylcholine applied to wild-type α7, or the T6′F or T6′S mutant receptors show that both wild-type α7 and the T6′S mutant respond poorly through a full range of concentrations, while succinylcholine is a relatively potent, full agonist of the T6′F mutant (FIG. 7).

All patents, patent applications, provisional applications, and publications referred to or cited herein, whether supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for identifying agents that modulate nicotinic acetylcholine receptor (nAChR) activity, comprising:
(a) providing a host cell that expresses a polynucleotide encoding a variant α7 nAChR polypeptide having a TM2 domain, wherein the variant α7 nAChR polypeptide has a serine residue in the sixth amino acid position of the TM2 domain;
(b) contacting a test agent with the host cell; and
(c) determining:
(i) the effect of the test agent on the variant α7 nAChR polypeptide or the host cell expressing the polynucleotide, or
(ii) the binding of the test agent to the host cell or the variant α7 nAChR polypeptide, or
(iii) both (c) (i) and (c) (ii).

2. The method of claim 1, wherein the TM2 domain comprises the amino acid sequence of SEQ ID NO:2.

3. The method of claim 1, wherein the polynucleotide comprises a nucleic acid sequence selected from the group consisting of SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

4. The method of claim 1, wherein the variant α7 nAChR polypeptide comprises a mammalian α7 nAChR polypeptide having a serine in the sixth amino acid position of the TM2 domain.

5. The method of claim 1, wherein the effect of the test agent on the α7 receptor activity of the variant α7 nAChR polypeptide is determined.

6. The method of claim 1, wherein the variant α7 nAChR polypeptide is a mammalian α7 nAChR polypeptide having a threonine to serine (T→S) substitution in the sixth amino acid position of the TM2 domain.

7. The method of claim 1, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

8. The method of claim 1, wherein the variant α7 nAChR polypeptide comprises the human α7 nAChR polypeptide having a threonine to serine (T→S) substitution in the sixth amino acid position of the TM2 domain.

9. The method of claim 1, wherein said determining of step (c) (ii) is performed by measuring a signal generated by a detectable moiety.

10. The method of claim 9, wherein the detectable moiety is selected from the group consisting of a fluorescent label, a radiolabel, a chemiluminescent label, and an enzyme.

11. The method of claim 1, wherein said determining of step (c) (i) is performed by measuring a signal generated by a radiolabeled ion, a fluorescent probe, or an electrical current.

12. The method of claim 1, wherein the host cell is selected from the group consisting of a bacterial cell, a mammalian cell, a yeast cell, an amphibian cell, and a starfish cell.
(ii) the binding of the test agent to the host cell or the variant α7 nAChR polypeptide, or
(iii) both of the foregoing.

13. The method of claim 1, wherein the host cell is a mammalian cell, and wherein the polynucleotide is stably expressed in the mammalian cell such that a functional receptor is formed, wherein the functional receptor elicits electrophysiological currents in vitro or in vivo when stimulated by a wild-type α7 agonist.

14. The method of claim 1, wherein the serine residue is a substitution for a threonine residue (T→S).

15. A method for identifying a cytoprotective agent, comprising:
(a) providing a host cell that expresses a polynucleotide encoding a variant α7 nAChR polypeptide having a TM2 domain, wherein the variant α7 nAChR polypeptide has a serine residue in the sixth amino acid position of the TM2 domain;
(b) contacting a test agent with the host cell; and
(c) monitoring the cell or cellular function for an indication of cytotoxicity.

16. The method of claim 15, wherein the TM2 domain comprises the amino acid sequence of SEQ ID NO:2.

17. The method of claim 15, wherein the polynucleotide comprises SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

18. The method of claim 15, wherein the host cell is selected from the group consisting of a bacterial cell, a mammalian cell, a yeast cell, an amphibian cell, and a starfish cell.

19. The method of claim 15, wherein the host cell is a mammalian cell, and wherein the polynucleotide is stably expressed in the mammalian cell such that a functional receptor is formed, wherein the functional receptor elicits electrophysiological currents in vitro or in vivo when stimulated by a wild-type α7 agonist.

20. The method of claim 15, wherein the serine residue is a substitution for a threonine residue (T→S).

21. The method of claim 15, wherein the variant α7 nAChR polypeptide comprises a mammalian α7 nAChR polypeptide having a serine in the sixth amino acid position of the TM2 domain.

22. The method of claim 15, wherein the variant α7 nAChR polypeptide is a mammalian α7 nAChR polypeptide having a threonine to serine (T→S) substitution in the sixth amino acid position of the TM2 domain.

23. The method of claim 15, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

24. The method of claim 15, wherein the variant α7 nAChR polypeptide comprises the human α7 nAChR polypeptide having a threonine to serine (T→S) substitution in the sixth amino acid position of the TM2 domain.

25. The method of claim 15, wherein the host cell is maintained in the presence of a substance so as to minimize or block a cytotoxic effect on the host cell.

26. An isolated polynucleotide encoding a variant α7 nAChR polypeptide having a TM2 domain, wherein the variant α7 nAChR polypeptide has a serine residue in the sixth amino acid position of the TM2 domain.

27. The isolated polynucleotide of claim 26, wherein the polypeptide comprises the amino acid sequence of SEQ ID NO:2.

28. The isolated polynucleotide of claim 26, wherein the serine residue is a substitution for a threonine residue (T→S).

29. The isolated polynucleotide of claim 26, wherein the variant α7 nAChR polypeptide comprises a mammalian α7 nAChR polypeptide having a serine in the sixth amino acid position of the TM2 domain.

30. The isolated polynucleotide of claim 26, wherein the variant α7 nAChR polypeptide is a mammalian α7 nAChR polypeptide having a threonine to serine (T→S) substitution in the sixth amino acid position of the TM2 domain.

31. The isolated polynucleotide of claim 26, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

32. The isolated polynucleotide of claim 26, wherein the variant α7 nAChR polypeptide comprises the human α7 nAChR polypeptide having a threonine to serine (T→S) substitution in the sixth amino acid position of the TM2 domain.

33. The isolated polynucleotide of claim 26, wherein the polynucleotide comprises SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

34. An isolated variant α7 nAChR polypeptide having a TM2 domain, wherein the variant α7 nAChR polypeptide has a serine residue in the sixth amino acid position of the TM2 domain.

35. The isolated polypeptide of claim 34, wherein the polypeptide comprises the amino acid sequence of SEQ ID NO:2.

36. The isolated polypeptide of claim 34, wherein the serine residue is a substitution for a threonine residue (T→S).

37. The isolated polypeptide of claim 34, wherein the variant α7 nAChR polypeptide comprises a mammalian α7 nAChR polypeptide having a serine in the sixth amino acid position of the TM2 domain.

38. The isolated polypeptide of claim 34, wherein the variant α7 nAChR polypeptide is a mammalian α7 nAChR polypeptide having a threonine to serine (T→S) substitution in the sixth amino acid position of the TM2 domain.

39. The isolated polypeptide of claim 34, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

40. The isolated polypeptide of claim 34, wherein the variant α7 nAChR polypeptide comprises the human α7 nAChR polypeptide having a threonine to serine (T→S) substitution in the sixth amino acid position of the TM2 domain.

41. An expression vector comprising a polynucleotide encoding a variant α7 nAChR polypeptide having a TM2 domain, wherein the variant α7 nAChR polypeptide has a serine residue in the sixth amino acid position of the TM2 domain, and wherein said polynucleotide is operably linked to a control sequence that directs the expression of said polynucleotide whereby said polynucleotide is expressed in a host cell.

42. The expression vector of claim 41, wherein said polypeptide encoded by said polynucleotide comprises the amino acid sequence of SEQ ID NO:2.

43. The expression vector of claim 41, wherein the serine residue is a substitution for a threonine residue (T→S).

44. The expression vector of claim 41, wherein the variant α7 nAChR polypeptide comprises a mammalian α7 nAChR polypeptide having a serine in the sixth amino acid position of the TM2 domain.

45. The expression vector of claim 41, wherein the variant α7 nAChR polypeptide is a mammalian α7 nAChR polypeptide having a threonine to serine (T→S) substitution in the sixth amino acid position of the TM2 domain.

46. The expression vector of claim 41, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

47. The expression vector of claim 41, wherein the variant α7 nAChR polypeptide comprises the human α7 nAChR polypeptide having a threonine to serine (T→S) substitution in the sixth amino acid position of the TM2 domain.

48. The expression vector of claim 41, wherein the polynucleotide comprises SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

49. The expression vector of claim 41, wherein said control sequence comprises an inducible promoter.

50. An isolated host cell comprising a polynucleotide encoding a variant α7 nAChR polypeptide having a TM2 domain, wherein the variant α7 nAChR polypeptide has a serine residue in the sixth amino acid position of the TM2 domain, and wherein said polynucleotide is operably linked to a control sequence that directs the expression of said polynucleotide in said host cell.

51. The isolated host cell of claim 50, wherein said host cell further comprises a control sequence operably linked to said polynucleotide, wherein the control sequence directs expression of said polynucleotide in said host cell.

52. The isolated host cell of claim 50, wherein said polynucleotide encodes a polypeptide comprising the amino acid sequence of SEQ ID NO:2.

53. The isolated host cell of claim 50, wherein the serine residue is a substitution for a threonine residue (T→S).

54. The isolated host cell of claim 50, wherein the variant α7 nAChR polypeptide comprises a mammalian α7 nAChR polypeptide having a serine in the sixth amino acid position of the TM2 domain.

55. The isolated host cell of claim 50, wherein the variant α7 nAChR polypeptide is a mammalian α7 nAChR polypeptide having a threonine to serine (T→S) substitution in the sixth amino acid position of the TM2 domain.

56. The isolated host cell of claim 50, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

57. The isolated host cell of claim 50, wherein the variant α7 nAChR polypeptide comprises the human α7 nAChR polypeptide having a threonine to serine (T→S) substitution in the sixth amino acid position of the TM2 domain.

58. The isolated host cell of claim 50, wherein the polynucleotide comprises SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

59. The isolated host cell of claim 50, wherein said cell is selected from the group consisting of a bacterial cell, a mammalian cell, a yeast cell, and an amphibian cell.

60. The isolated host cell of claim 50, wherein said cell is a human cell.

61. The isolated host cell of claim 50, wherein said host cell is a mammalian cell, and wherein said polynucleotide is stably expressed in said mammalian cell such that a functional receptor is formed, wherein said functional receptor elicits electrophysiological currents in vitro or in vivo when stimulated by a wild-type α7 agonist.

62. A method for treating a neurological condition in a patient, said method comprising administering a polypeptide to the patient having a TM2 domain, wherein the polypeptide has a serine residue in the sixth amino acid position of the TM2 domain; or administering a polynucleotide encoding the polypeptide to the patient, wherein the polynucleotide is expressed within the patient.

63. The method of claim 62, wherein the TM2 domain comprises the amino acid of SEQ ID NO:2.

64. The method of claim 62, wherein a vector or host cell comprising the polynucleotide is administered to the patient.

65. The method of claim 62, wherein a viral vector or non-viral vector comprising the polynucleotide is administered to the patient.

66. The method of claim 62, wherein the serine residue is a substitution for a threonine residue (T→S).

67. The method of claim 62, wherein the variant α7 nAChR polypeptide comprises a mammalian α7 nAChR polypeptide having a serine in the sixth amino acid position of the TM2 domain.

68. The method of claim 62, wherein the variant α7 nAChR polypeptide is a mammalian α7 nAChR polypeptide having a threonine to serine (T→S) substitution in the sixth amino acid position of the TM2 domain.

69. The method of claim 62, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

70. The method of claim 62, wherein the variant α7 nAChR polypeptide comprises the human α7 nAChR polypeptide having a threonine to serine (T→S) substitution in the sixth amino acid position of the TM2 domain.

71. The method of claim 62, wherein the polynucleotide comprises SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

72. A composition comprising a variant α7 nAChR polypeptide having a TM2 domain, wherein the polypeptide has a serine residue in the sixth amino acid position of the TM2 domain; or a polynucleotide encoding said polypeptide; and a pharmaceutically acceptable carrier.

73. The composition of claim 72, wherein the TM2 domain comprises the amino acid of SEQ ID NO:2.

74. The composition of claim 72, wherein the serine residue is a substitution for a threonine residue (T→S).

75. The composition of claim 72, wherein the variant α7 nAChR polypeptide comprises a mammalian α7 nAChR polypeptide having a serine in the sixth amino acid position of the TM2 domain.

76. The composition of claim 72, wherein the variant α7 nAChR polypeptide is a mammalian α7 nAChR polypeptide having a threonine to serine (T→S) substitution in the sixth amino acid position of the TM2 domain.

77. The composition of claim 72, wherein the variant α7 nAChR polypeptide is an α7 nAChR polypeptide of human, non-human primate, rodent, chicken, or zebrafish, having a threonine to serine (T→S) substitution in the sixth amino acid position of the TM2 domain.

78. The composition of claim 72, wherein the variant α7 nAChR polypeptide comprises the human α7 nAChR polypeptide having a threonine to serine (T→S) substitution in the sixth amino acid position of the TM2 domain.

79. The composition of claim 72, wherein the polynucleotide comprises SEQ ID NO: 18, SEQ ID NO:19, SEQ ID NO:21, SEQ ID NO:22, SEQ ID NO:23 SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, and SEQ ID NO:60, or degenerate variants of any of the foregoing.

80. A method for identifying α7 nAChR modulators, said method comprising: (a) providing a variant α7 nAChR polypeptide having a TM2 domain, wherein the polypeptide has a serine residue in the sixth amino acid position of the TM2 domain; (b) contacting the polypeptide with an Aβ peptide capable of interacting with the polypeptide; (c) exposing the polypeptide and the Aβ peptide to a test agent; and (d) determining the effect of the test agent on the interaction of the polypeptide and the Aβ peptide.

81. The method of claim 80, wherein the TM2 domain comprises the amino acid sequence of SEQ ID NO:2.

82. The method of claim 80, wherein the Aβ peptide is comprises β-amyloid peptide₁₋₄₂, β-amyloid₁₋₄₀, or a functional fragment or variant thereof.

83. A method for identifying a cytoprotective agent, comprising:
(a) providing a host cell that expresses a polynucleotide encoding a variant α7 nAChR polypeptide having a TM2 domain, wherein the variant α7 nAChR polypeptide has a serine residue in the sixth amino acid position of the TM2 domain;
(b) contacting a test agent with the host cell; and
(c) monitoring the cell or cellular function for overstimulation of the variant α7 nAChR polypeptide, wherein overstimulation of the variant α7 nAChR polypeptide indicates that the test agent is sufficient to activate a wild-type α7 receptor at a cytoprotective level.

84. The method of claim 83, wherein the TM2 domain comprises the amino acid sequence of SEQ ID NO:2.


SEQUENCE LISTING (60 sequences)

SEQ ID NO: 1 - protein, 20 aa, Homo sapiens
  MISC_FEATURE (1)..(20): wild-type TM2 domain
ISLGITVLLSLTVFMLLVAE

SEQ ID NO: 2 - protein, 20 aa, Artificial Sequence
  T6′S mutant TM2 domain
ISLGISVLLSLTVFMLLVAE

SEQ ID NO: 3 - DNA, 60 bp, Rattus norvegicus
  misc_feature (1)..(60): sequence encoding wild-type rat TM domain
atctctcttggaataactgtcttactttctctgactgtcttcatgctgcttgtggctgag

SEQ ID NO: 4 - DNA, 1509 bp, Rattus norvegicus
  CDS (1)..(1509)
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaataactgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 5 - protein, 502 aa, Rattus norvegicus
  SIGNAL (1)..(22)
MCGGRGGIWLALAAALLHVSLQGEFQRRLYKELVKNYNPLERPVANDSQPLTVYFSLSLLQIMDVDEKNQVLTTNIWLQMSWTDHYLQWNMSEYPGVKNVRFPDGQIWKPDILLYNSADERFDATFHTNVLVNASGHCQYLPPGIFKSSCYIDVRWFPFDVQQCKLKFGSWSYGGWSLDLQMQEADISSYIPNGEWDLMGIPGKRNEKFYECCKEPYPDVTYTVTMRRRTLYYGLNLLIPCVLISALALLVFLLPADSGEKISLGITVLLSLTVFMLLVAEIMPATSDSVPLIAQYFASTMIIVGLSVVVTVIVLRYHHHDPDGGKMPKWTRIILLNWCAWFLRMKRPGEDKVRPACQHKPRRCSLASVELSAGAGPPTSNGNLLYIGFRGLEGMHCAPTPDSGVVCGRLACSPTHDEHLMHGAHPSDGDPDLAKILEEVRYIANRFRCQDESEVICSEWKFAACVVDRLCLMAFSVFTIICTIGILMSAPNFVEAVSKDFA

SEQ ID NO: 6 - DNA, 1509 bp, Homo sapiens
  CDS (1)..(1509)
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaattcttctgggcattgccagtacctgcctccaggcatattcaagagttcctgctacatcgatgtacgctggtttccctttgatgtgcagcactgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggacgctctactatggcctcaacctgctgatcccctgtgtgctcatctccgccctcgccctgctggtgttcctgcttcctgcagattccggggagaagatttccctggggataacagtcttactctctcttaccgtcttcatgctgctcgtggctgagatcatgcccgcaacatccgattcggtaccattgatagcccagtacttcgccagcaccatgatcatcgtgggcctctcggtggtggtgacggtgatcgtgctgcagtaccaccaccacgaccccgacgggggcaagatgcccaagtggaccagagtcatccttctgaactggtgcgcgtggttcctgcgaatgaagaggcccggggaggacaaggtgcgcccggcctgccagcacaagcagcggcgctgcagcctggccagtgtggagatgagcgccgtggcgccgccgcccgccagcaacgggaacctgctgtacatcggcttccgcggcctggacggcgtgcactgtgtcccgacccccgactctggggtagtgtgtggccgcatggcctgctcccccacgcacgatgagcacctcctgcacggcgggcaaccccccgagggggacccggacttggccaagatcctggaggaggtccgctacattgccaaccgcttccgctgccaggacgaaagcgaggcggtctgcagcgagtggaagttcgccgcctgtgtggtggaccgcctgtgcctcatggccttctcggtcttcaccatcatctgcaccatcggcatcctgatgtcggctcccaacttcgtggaggccgtgtccaaagactttgcgtaa

SEQ ID NO: 7 - protein, 502 aa, Homo sapiens
  SIGNAL (1)..(22)
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGVHCVPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 8 - DNA, 1509 bp, Mus musculus
  CDS (1)..(1509)
atgtgcggccggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagcctcctgcagatcatggatgtggatgagaagaaccaagttttaaccaccaacatttggcttcaaatgtcttggacagatcactatttgcagtggaacatgtctgagtaccccggagtgaaaaatgttcgttttccagatggccagatttggaaaccagacattctcctctataacagtgcagatgaacgctttgatgccacattccacaccaacgtcttggtgaatgcatctgggcattgccagtatctccctccaggcatattcaagagctcctgctacatcgatgtacgctggttcccttttgatgtgcagcagtgcaaactgaagtttgggtcctggtcctatggagggtggtccttggacctgcagatgcaagaggcagatatcagcagctatatccccaatggagaatgggatctcatgggaatccctggcaaaaggaatgagaagttctatgaatgctgcaaagagccatacccagatgtcacctacacagtaaccatgcgccgtaggacactctactatggcctcaacctgctcattccttgtgtgctcatttcagccctggctttgctggtattcttgctgcctgcagactctggagagaaaatctctcttggaataactgtcttactttctctgactgtcttcatgctgcttgtggctgagatcatgccagcaacatctgattccgtgcccttgatagcacagtacttcgccagcaccatgatcatcgtgggcctctcagtggtcgtgacagtgattgtgctgcgatatcaccaccatgaccctgatggtggcaaaatgcctaagtggaccaggatcattcttctgaattggtgtgcatggtttctgcgcatgaagaggccgggagaggacaaggtgcggccggcttgtcagcacaagcctcggcgctgcagcctggccagtgtggagctgagcgcaggtgcggggccaccctccagcaatggcaacctgctctacattggcttccgaggcctggagggcatgcactgtgccccaactccagactctggggtcgtgtgtggtcgtttggcctgctccccaacacatgatgagcacctcatgcatggtacacacccctctgatggggaccccgacctggccaagatcctggaggaggtccgctacatcgccaaccgcttccgctgccaggatgagagtgaggtgatctgcagtgagtggaagtttgcggcctgtgtggtggaccgcttgtgcctcatggccttttcggtttttaccatcatctgcaccatcggcatcctcatgtcagccccaaactttgtggaggctgtgtccaaagactttgcttaa

SEQ ID NO: 9 - protein, 502 aa, Mus musculus
  SIGNAL (1)..(22)
MCGRRGGIWLALAAALLHVSLQGEFQRRLYKELVKNYNPLERPVANDSQPLTVYFSLSLLQIMDVDEKNQVLTTNIWLQMSWTDHYLQWNMSEYPGVKNVRFPDGQIWKPDILLYNSADERFDATFHTNVLVNASGHCQYLPPGIFKSSCYIDVRWFPFDVQQCKLKFGSWSYGGWSLDLQMQEADISSYIPNGEWDLMGIPGKRNEKFYECCKEPYPDVTYTVTMRRRTLYYGLNLLIPCVLISALALLVFLLPADSGEKISLGITVLLSLTVFMLLVAEIMPATSDSVPLIAQYFASTMIIVGLSVVVTVIVLRYHHHDPDGGKMPKWTRIILLNWCAWFLRMKRPGEDKVRPACQHKPRRCSLASVELSAGAGPPSSNGNLLYIGFRGLEGMHCAPTPDSGVVCGRLACSPTHDEHLMHGTHPSDGDPDLAKILEEVRYIANRFRCQDESEVICSEWKFAACVVDRLCLMAFSVFTIICTIGILMSAPNFVEAVSKDFA

SEQ ID NO: 10 - DNA, 1509 bp, Macaca mulatta
  CDS (1)..(1509)
atgcgctgctcgcagggaggcgtctggctggctctggccgcgtcgctcctgcatgtgtccctgcaaggcgagttccagaggaagctttacaaggagctggtcaagaactacaaccccttggagaggcccgtggccaatgactcgcaaccgctcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgcggatgagcgctttgacgccacattccacaccaacgtgttggtgaattcttctgggcattgccagtacctgcctccaggcatattcaagagttcctgctacatcgatgtgcgctggtttccctttgatgtgcagcattgcaaactgaagtttggatcctggtcttatggaggctggtccttggatctgcagatgcaggaggcagatatcagtggctatatccccagtggagaatgggacctagtgggaattcccggcaagaggagtgaaaagttctatgagtgctgcaaagagccctaccccgatgtcaccttcacagtgaccatgcgccgcaggaccctctactacggcctcaacctgctgatcccctgtgtgctcatctctgcccttgccctgctggtgttcctgcttcctgcagattccggggagaagatttccctggggataacagtcttactctctctcactgtcttcatgctgctcgtggctgagatcatgcccgcaacatctgattcagtaccattgatagcccagtacttcgccagcaccatgatcatcgtgggcctctccgtggtggtgacggtgatcgtgctgcagtaccaccaccacgaccccgacgggggcaagatgcccaagtggaccagagtcatccttctgaactggtgcgcgtggttcctgcgcatgaagaggccgggagaggataaggtgcgcccggcctgccagcacaagcagcgccgctgcagcctggccagcgtggagatgagcgccgtggcgccgccgcccgccagcaacgggaacctgctgtacatcggcttccgcggcctggacggcatgcattgcgccccgacccccgactccggggtggtgtgcggccgcatggcctgctcccccacgcacgacgagcacctcctgcacggtgggcagccccccgagggggacccggacctggccaagatcctggaggaggtccgctacatcgccaaccgctttcgctgccaggacgaaagcgaggcggtctgcagtgagtggaagttcgccgcctgcgtggtggaccgcctgtgcctcatggccttctcggtcttcaccatcatctgcaccatcggcatcctgatgtcggctcccaacttcgtggaggccgtgtccaaagactttgcgtaa

SEQ ID NO: 11 - protein, 502 aa, Macaca mulatta
  SIGNAL (1)..(22)
MRCSQGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPSGEWDLVGIPGKRSEKFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGMHCAPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 12 - DNA, 1500 bp, Bos taurus
  CDS (1)..(1500)
atgcgcggctcgttgtgcctggcgctggcagcgtcgatcttgcacgtgtccctgcaaggcgagttccagaggaagctctacaaagacctggtgaagaactacaaccccttggagaggccggtggccaacgactcgctgccgctcacagtctacttctccctgagcctcctgcagatcatggacgtggatgagaagaaccaggtgttgaccaccaacatttggctacaaatgacttggacagatcactatttacagtggaatgcatccgagtatccaggcgtgaagacggttcgtttcccagacggccagatctggaagccagacattctcctctacaacagtgctgatgaacggtttgatgctacgttccacaccaatgtgttagtgaattcttctgggcactgccagtacctccctccaggcatctttaagagctcctgctacatcgatgtgcgctggttcccatttgatgtgcagcagtgcaaactgaagtttgggtcctggtcttatggaggatggtccttggatctgcaaatgcaggaggcagacatcagcggctatatcccaaatggagaatgggaccttgtgggggtcttgggcaagcggagtgagaagttctacgagtgctgcaaagaaccctacccagacgtgaccttcacggtcagcatccgccgcaggactctctactacggcctcaacctgctcatcccctgtgtgctcatctctgccctagccctcctcgtcttcttgctccctgcagactctggagagaaaatctccctgggaatcacagttttactctccctcaccgtcttcatgctgctggtagctgagatcatgcctgcgacctctgactcagtgcccttaatagcccagtacttcgccagcaccatgatcatcgtgggcctctctgtggttgtcacggtgatcgtgctacagtaccaccaccacgaccctgatggtggcaagatgcccaagtggaccagagtcgtcctcctgaactggtgtgcctggttccttcgcatgaagaggccgggggaggacaaggtgcggccagcctgccagcacaacgagcggcgctgcagcctggccagcgtggagatgagcgcagtggcggggccgccggccaccaatggcaacctgctgtacatcggctttcgcggcctggacaccatgcactgcgcacccacccctgactcaggggtcgtgtgcggccgcgtggcctgctccccaacccatgacgagcacctgctgcacgccggccagccctctgagggggacccggacctagccaagatcctggaggaggtgcgctacattgcccaccgcttccgctgccaggatgagagtgaggccgtgtgcagtgagtggaagttcgcggcctgtgtggtggaccgcctgtgtctcatggccttctccgtcttcaccatcctctgcaccattggcatcctgatgtcggcgcccaacttcgttgaagctgtgtccaaagacttcgcttga

SEQ ID NO: 13 - protein, 499 aa, Bos taurus
  SIGNAL (1)..(19)
MRGSLCLALAASILHVSLQGEFQRKLYKDLVKNYNPLERPVANDSLPLTVYFSLSLLQIMDVDEKNQVLTTNIWLQMTWTDHYLQWNASEYPGVKTVRFPDGQIWKPDILLYNSADERFDATFHTNVLVNSSGHCQYLPPGIFKSSCYIDVRWFPFDVQQCKLKFGSWSYGGWSLDLQMQEADISGYIPNGEWDLVGVLGKRSEKFYECCKEPYPDVTFTVSIRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVVLLNWCAWFLRMKRPGEDKVRPACQHNERRCSLASVEMSAVAGPPATNGNLLYIGFRGLDTMHCAPTPDSGVVCGRVACSPTHDEHLLHAGQPSEGDPDLAKILEEVRYIAHRFRCQDESEAVCSEWKFAACVVDRLCLMAFSVFTILCTIGILMSAPNFVEAVSKDFA

SEQ ID NO: 14 - DNA, 1509 bp, Gallus gallus
  CDS (1)..(1509)
atgggcctccgggcgctgatgctgtggctgctggcggcggcggggctcgtgcgcgagtccctgcaaggagagttccaaaggaagctgtacaaggagctgctgaagaactacaaccctctggaacgaccagttgcaaatgactcccagccgctcactgtctatttcactctcagcctcatgcagatcatggatgtggatgaaaagaatcaagtattaacaacaaacatctggctacaaatgtactggacagatcattacttacagtggaatgtgtctgaataccctggagtgaagaacgtccgttttcctgatggactgatttggaagccagatattcttctctataacagtgctgatgaaagatttgatgctacatttcacactaatgttttagtcaattcttcgggacactgccaatatctgccaccaggcatatttaaaagctcatgctacatagacgtgcgttggtttccatttgatgttcagaagtgcaatctgaagtttggatcttggacatatggaggctggtccttagacttacaaatgcaagaagcagatatatccggctatatttcaaatggagagtgggatttagtaggaattcctgggaagagaactgagagcttttatgagtgctgtaaagaaccatacccagatatcacattcacagtaaccatgagacgcagaactctctactacgggctcaaccttcttattccctgtgtactgatatcagcacttgccttattagtctttctgcttccagcagactcaggagaaaagatctcactaggtataacagttttattgtctctcaccgtcttcatgttactcgtggctgaaattatgccagcaacatctgattctgtgcccttaattgctcagtattttgccagcaccatgattattgttggcctctctgttgttgtcactgttatcgttctacaataccatcatcacgatccagatgggggaaaaatgcctaaatggacaagagtcatccttctgaattggtgtgcttggtttctgaggatgaagagaccaggggaagataaagtgcgtcccgcctgtcaacataaacagcgccgatgcagcctgtcaagcatggagatgaacactgtgagtggtcagcaatgcagtaatgggaacatgctgtatattgggtttcgagggctggatggggttcactgcacacccaccactgattcaggggtgatctgtgggaggatgacctgttcaccaacagaggaagaaaatcttctgcacagtggccacccctctgaaggcgacccagatttggctaagatcctggaagaggtcagatacattgcaaacaggttcagagaccaggatgaagaagaagccatttgcaacgaatggaagtttgcagcctctgtagtagatcggctctgcttgatggcattttcggtcttcaccatcatttgtacaattggcatcttaatgtcagcaccaaactttgtagaggctgtctctaaagattttgcttaa

SEQ ID NO: 15 - protein, 502 aa, Gallus gallus
  SIGNAL (1)..(22)
MGLRALMLWLLAAAGLVRESLQGEFQRKLYKELLKNYNPLERPVANDSQPLTVYFTLSLMQIMDVDEKNQVLTTNIWLQMYWTDHYLQWNVSEYPGVKNVRFPDGLIWKPDILLYNSADERFDATFHTNVLVNSSGHCQYLPPGIFKSSCYIDVRWFPFDVQKCNLKFGSWTYGGWSLDLQMQEADISGYISNGEWDLVGIPGKRTESFYECCKEPYPDITFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLSSMEMNTVSGQQCSNGNMLYIGFRGLDGVHCTPTTDSGVICGRMTCSPTEEENLLHSGHPSEGDPDLAKILEEVRYIANRFRDQDEEEAICNEWKFAASVVDRLCLMAFSVFTIICTIGILMSAPNFVEAVSKDFA

SEQ ID NO: 16 - DNA, 1530 bp, Danio rerio
  CDS (1)..(1530)
atgggaatttgggaatatactctttatttcaccaccacatgctgcctatggagtgtgtcacttcagggggagcatcagaggagactctatagagacctgatgaaagattataacccactggagagacctgtgttcaatgacacccactcactcacggtgtatttcagcatgagcctcatgcagattatggatgtggatgaaaagaatcaagttcttacgaccaacatttggctacagctgtactggtatgactactatcttcaatggaacgcctcagaatatcctggagtaaccaatgtaaggtttcctgacagtcagatctggaagcctgacatcttactttataacagtgctgatgaaaggtttgatgccacatttcatactaatgtgctggtgaactcttctggtgcctgccaatacttaccgccagggatcttcaaaagcacctgctacatcgacgttcgctggttcccctttgatcttcagaggtgtgatctgaaatttggctcctggacgtatggaggatggtctctggacctgcagatgattgatgccgacatcacaggatacattgctaatggagagtgggaccttgtggaagttccaggtagacggaatgagaggttctacgactgctgcaaggagccgtacccagatgtgacttttacggtggtgatgcggagacgaacactgtattacggtcttaatctacttattccctgtgtgctcatctctactctggccctgctggtgttcctgctgcctgctgactcaggagaaaagatctcgctcgggatcaccgtcctgctctcgctgacagttttcatgcttttggtagcagagataatgccggcaacatctgactctgtgcctttaatagctcagtattttgccaccaccatggttattgttggactatctgtaatagccacagtctgggttttacaataccactaccatgatcctgaaggaggaaaaatgccaaaatggacacgtgtggtgctgttgaactggtgtgcctggttcctgcggatgaagagacctggcgaagacaaagttcgcccagcttgtcacaacaagcagccgcgcagcagcctctccagcgtggatctaaacatcagccctggagtcgcacagtccaccaatggaaacctgctgtacatcggcttccgcgggatggacactattcactatgctacttcacccgattctggggtgatctgcagccggcttgtggcgaccggagaggaggatgtgctgctccccggagcccaggcatcttcagtcagtagtagcggccctggagaaaccgaactgtccaagatcttggatgaggtgcggtacatttccaagcgctttcgggaccaagacgaggaagacactgtgtgtaatgagtggaagtttgcagcgtctgtgattgaccgtctctgtctcatggctttttccctgttcaccatcctctgcaccattggtatcttaatgtctgcccctaactttgttgaggccatatcaaaagattttttcacctga

SEQ ID NO: 17 - protein, 509 aa, Danio rerio
  SIGNAL (1)..(22)
MGIWEYTLYFTTTCCLWSVSLQGEHQRRLYRDLMKDYNPLERPVFNDTHSLTVYFSMSLMQIMDVDEKNQVLTTNIWLQLYWYDYYLQWNASEYPGVTNVRFPDSQIWKPDILLYNSADERFDATFHTNVLVNSSGACQYLPPGIFKSTCYIDVRWFPFDLQRCDLKFGSWTYGGWSLDLQMIDADITGYIANGEWDLVEVPGRRNERFYDCCKEPYPDVTFTVVMRRRTLYYGLNLLIPCVLISTLALLVFLLPADSGEKISLGITVLLSLTVFMLLVAEIMPATSDSVPLIAQYFATTMVIVGLSVIATVWVLQYHYHDPEGGKMPKWTRVVLLNWCAWFLRMKRPGEDKVRPACHNKQPRSSLSSVDLNISPGVAQSTNGNLLYIGFRGMDTIHYATSPDSGVICSRLVATGEEDVLLPGAQASSVSSSGPGETELSKILDEVRYISKRFRDQDEEDTVCNEWKFAASVIDRLCLMAFSLFTILCTIGILMSAPNFVEAISKDFFT

SEQ ID NO: 18 - DNA, 60 bp, Artificial Sequence
  degenerate variant encoding T6′S mutant TM2 domain
atctctcttggaatatctgtcttactttctctgactgtcttcatgctgcttgtggctgag

SEQ ID NO: 19 - DNA, 1509 bp, Artificial Sequence
  degenerate variant encoding T6′S mutant alpha7 subunit
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaatatctgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 20 - protein, 502 aa, Artificial Sequence
  T6′S mutant alpha7 subunit
MCGGRGGIWLALAAALLHVSLQGEFQRRLYKELVKNYNPLERPVANDSQPLTVYFSLSLLQIMDVDEKNQVLTTNIWLQMSWTDHYLQWNMSEYPGVKNVRFPDGQIWKPDILLYNSADERFDATFHTNVLVNASGHCQYLPPGIFKSSCYIDVRWFPFDVQQCKLKFGSWSYGGWSLDLQMQEADISSYIPNGEWDLMGIPGKRNEKFYECCKEPYPDVTYTVTMRRRTLYYGLNLLIPCVLISALALLVFLLPADSGEKISLGISVLLSLTVFMLLVAEIMPATSDSVPLIAQYFASTMIIVGLSVVVTVIVLRYHHHDPDGGKMPKWTRIILLNWCAWFLRMKRPGEDKVRPACQHKPRRCSLASVELSAGAGPPTSNGNLLYIGFRGLEGMHCAPTPDSGVVCGRLACSPTHDEHLMHGAHPSDGDPDLAKILEEVRYIANRFRCQDESEVICSEWKFAACVVDRLCLMAFSVFTIICTIGILMSAPNFVEAVSKDFA

SEQ ID NO: 21 - DNA, 60 bp, Artificial Sequence
  degenerate variant encoding T6′S mutant TM2 domain
atctctcttggaatatccgtcttactttctctgactgtcttcatgctgcttgtggctgag

SEQ ID NO: 22 - DNA, 60 bp, Artificial Sequence
  degenerate variant encoding T6′S mutant TM2 domain
atctctcttggaatatcagtcttactttctctgactgtcttcatgctgcttgtggctgag

SEQ ID NO: 23 - DNA, 60 bp, Artificial Sequence
  degenerate variant encoding T6′S mutant TM2 domain
atctctcttggaatatcggtcttactttctctgactgtcttcatgctgcttgtggctgag

SEQ ID NO: 24 - DNA, 60 bp, Artificial Sequence
  degenerate variant encoding T6′S mutant TM2 domain
atctctcttggaataagtgtcttactttctctgactgtcttcatgctgcttgtggctgag

SEQ ID NO: 25 - DNA, 60 bp, Artificial Sequence
  degenerate variant encoding T6′S mutant TM2 domain
atctctcttggaataagcgtcttactttctctgactgtcttcatgctgcttgtggctgag

SEQ ID NO: 26 - DNA, 1509 bp, Artificial Sequence
  degenerate variant encoding T6′S mutant alpha7 subunit
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaatatccgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 27 - DNA, 1509 bp, Artificial Sequence
  degenerate variant encoding T6′S mutant alpha7 subunit
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaatatcagtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 28 - DNA, 1509 bp, Artificial Sequence
  degenerate variant encoding T6′S mutant alpha7 subunit
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaatatcggtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 29 - DNA, 1509 bp, Artificial Sequence
  degenerate variant encoding T6′S mutant alpha7 subunit
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaataagtgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 30 - DNA, 1509 bp, Artificial Sequence
  degenerate variant encoding T6′S mutant alpha7 subunit
atgtgcggcgggcggggaggcatctggctggctctggccgcggcgctgctgcacgtgtccctgcaaggcgagttccagaggaggctgtacaaggagctggtcaagaactacaacccgctggagaggccggtggccaacgactcgcagccgctcaccgtgtacttctccctgagtctcctgcagatcatggatgtggatgagaagaaccaagttttaaccaccaacatttggctacaaatgtcttggacagatcactatttgcagtggaacatgtctgagtaccccggagtgaagaatgttcgttttccagatggccagatttggaaaccagacattctcctctataacagtgctgatgagcgctttgatgccacgttccacaccaatgttttggtgaatgcatctgggcattgccagtatctccctccaggcatattcaagagctcctgctacattgacgttcgctggttcccttttgatgtgcagcagtgcaaactgaagtttgggtcctggtcctatggagggtggtcactggacctgcaaatgcaagaggcagatatcagcagctatatccccaacggagaatgggatctcatgggaatccctggcaaaaggaatgagaagttctatgagtgctgcaaagagccatacccagatgtcacctacacagtaaccatgcgccgtaggacactctactatggcctcaatctgctcatcccttgtgtactcatttcagccctggctctgctggtattcttgctgcctgcagactctggagagaaaatctctcttggaataagcgtcttactttctctgactgtcttcatgctgcttgtggctgagatcatgccagcaacatctgattctgtgcccttgatagcacaatacttcgccagcaccatgatcatcgtgggcctctctgtagtggtgacagtgattgtgctgagatatcaccaccatgaccctgatggtggcaaaatgcctaagtggaccagaatcattctcctgaactggtgtgcatggtttctgcgcatgaagaggcccggagaggacaaggtgcggccagcttgtcagcacaagcctcggcgctgcagcctggccagtgtggagctgagtgcaggtgctgggccacccaccagcaatggcaacctgctctacattggcttccgaggcctggagggcatgcactgtgccccaactccagactctggggtcgtatgtggccgtttggcctgctccccaacacatgatgagcacctcatgcacggtgcacacccctctgatggggaccccgacctggccaagatcctggaggaggtccgctacatcgccaaccgcttccgctgccaggacgagagtgaggtgatctgcagtgaatggaagtttgcagcctgcgtggtggaccgcttgtgcctcatggccttttcggtctttaccatcatctgtaccatcggcatcctcatgtcagctccaaactttgtggaggctgtgtccaaagactttgcttaa

SEQ ID NO: 31 - DNA, 60 bp, Artificial Sequence
  degenerate variant encoding T6′S mutant TM2 domain
atttccctggggatatctgtcttactctctcttaccgtcttcatgctgctcgtggctgag

SEQ ID NO: 32 - DNA, 60 bp, Artificial Sequence
  degenerate variant encoding T6′S mutant TM2 domain
atttccctggggatatccgtcttactctctcttaccgtcttcatgctgctcgtggctgag

SEQ ID NO: 33 - DNA, 60 bp, Artificial Sequence
  degenerate variant encoding T6′S mutant TM2 domain
atttccctggggatatcagtcttactctctcttaccgtcttcatgctgctcgtggctgag

SEQ ID NO: 34 - DNA, 60 bp, Artificial Sequence
  degenerate variant encoding T6′S mutant TM2 domain
atttccctggggatatcggtcttactctctcttaccgtcttcatgctgctcgtggctgag

SEQ ID NO: 35 - DNA, 60 bp, Artificial Sequence
  degenerate variant encoding T6′S mutant TM2 domain
atttccctggggataagtgtcttactctctcttaccgtcttcatgctgctcgtggctgag

SEQ ID NO: 36 - DNA, 60 bp, Artificial Sequence
  degenerate variant encoding T6′S mutant TM2 domain
atttccctggggataagcgtcttactctctcttaccgtcttcatgctgctcgtggctgag

SEQ ID NO: 37 - DNA, 60 bp, Artificial Sequence
  degenerate variant encoding T6′S mutant TM2 domain
atttccctggggatatctgtcttactctctctcactgtcttcatgctgctcgtggctgag

SEQ ID NO: 38 - DNA, 60 bp, Artificial Sequence
  degenerate variant encoding T6′S mutant TM2 domain
atttccctggggatatccgtcttactctctctcactgtcttcatgctgctcgtggctgag

SEQ ID NO: 39 - DNA, 60 bp, Artificial Sequence
  degenerate variant encoding T6′S mutant TM2 domain
atttccctggggatatcagtcttactctctctcactgtcttcatgctgctcgtggctgag

SEQ ID NO: 40 - DNA, 60 bp, Artificial Sequence
  degenerate variant encoding T6′S mutant TM2 domain
atttccctggggatatcggtcttactctctctcactgtcttcatgctgctcgtggctgag

SEQ ID NO: 41 - DNA, 60 bp, Artificial Sequence
  degenerate variant encoding T6′S mutant TM2 domain
atttccctggggataagtgtcttactctctctcactgtcttcatgctgctcgtggctgag

SEQ ID NO: 42 - DNA, 60 bp, Artificial Sequence
  degenerate variant encoding T6′S mutant TM2 domain
atttccctggggataagcgtcttactctctctcactgtcttcatgctgctcgtggctgag

SEQ ID NO: 43 - DNA, 60 bp, Artificial Sequence
  degenerate variant encoding T6′S mutant TM2 domain
atctccctgggaatctctgttttactctccctcaccgtcttcatgctgctggtagctgag

SEQ ID NO: 44 - DNA, 60 bp, Artificial Sequence
  degenerate variant encoding T6′S mutant TM2 domain
atctccctgggaatctccgttttactctccctcaccgtcttcatgctgctggtagctgag

SEQ ID NO: 45 - DNA, 60 bp, Artificial Sequence
  degenerate variant encoding T6′S mutant TM2 domain
atctccctgggaatctcagttttactctccctcaccgtcttcatgctgctggtagctgag

SEQ ID NO: 46 - DNA, 60 bp, Artificial Sequence
  degenerate variant encoding T6′S mutant TM2 domain
atctccctgggaatctcggttttactctccctcaccgtcttcatgctgctggtagctgag

SEQ ID NO: 47 - DNA, 60 bp, Artificial Sequence
  degenerate variant encoding T6′S mutant TM2 domain
atctccctgggaatcagtgttttactctccctcaccgtcttcatgctgctggtagctgag

SEQ ID NO: 48 - DNA, 60 bp, Artificial Sequence
  degenerate variant encoding T6′S mutant TM2 domain
atctccctgggaatcagcgttttactctccctcaccgtcttcatgctgctggtagctgag

SEQ ID NO: 49 - DNA, 60 bp, Artificial Sequence
  degenerate variant encoding T6′S mutant TM2 domain
atctcactaggtatatctgttttattgtctctcaccgtcttcatgttactcgtggctgaa

SEQ ID NO: 50 - DNA, 60 bp, Artificial Sequence
  degenerate variant encoding T6′S mutant TM2 domain
atctcactaggtatatccgttttattgtctctcaccgtcttcatgttactcgtggctgaa

SEQ ID NO: 51 - DNA, 60 bp, Artificial Sequence
  degenerate variant encoding T6′S mutant TM2 domain
atctcactaggtatatcagttttattgtctctcaccgtcttcatgttactcgtggctgaa

SEQ ID NO: 52 - DNA, 60 bp, Artificial Sequence
  degenerate variant encoding T6′S mutant TM2 domain
atctcactaggtatatcggttttattgtctctcaccgtcttcatgttactcgtggctgaa

SEQ ID NO: 53 - DNA, 60 bp, Artificial Sequence
  degenerate variant encoding T6′S mutant TM2 domain
atctcactaggtataagtgttttattgtctctcaccgtcttcatgttactcgtggctgaa

SEQ ID NO: 54 - DNA, 60 bp, Artificial Sequence
  degenerate variant encoding T6′S mutant TM2 domain
atctcactaggtataagcgttttattgtctctcaccgtcttcatgttactcgtggctgaa

SEQ ID NO: 55 - DNA, 60 bp, Artificial Sequence
  degenerate variant encoding T6′S mutant TM2 domain
atctcgctcgggatctctgtcctgctctcgctgacagttttcatgcttttggtagcagag

SEQ ID NO: 56 - DNA, 60 bp, Artificial Sequence
  degenerate variant encoding T6′S mutant TM2 domain
atctcgctcgggatctccgtcctgctctcgctgacagttttcatgcttttggtagcagag

SEQ ID NO: 57 - DNA, 60 bp, Artificial Sequence
  degenerate variant encoding T6′S mutant TM2 domain
atctcgctcgggatctcagtcctgctctcgctgacagttttcatgcttttggtagcagag

SEQ ID NO: 58 - DNA, 60 bp, Artificial Sequence
  degenerate variant encoding T6′S mutant TM2 domain
atctcgctcgggatctcggtcctgctctcgctgacagttttcatgcttttggtagcagag

SEQ ID NO: 59 - DNA, 60 bp, Artificial Sequence
  degenerate variant encoding T6′S mutant TM2 domain
atctcgctcgggatcagtgtcctgctctcgctgacagttttcatgcttttggtagcagag

SEQ ID NO: 60 - DNA, 60 bp, Artificial Sequence
  degenerate variant encoding T6′S mutant TM2 domain
atctcgctcgggatcagcgtcctgctctcgctgacagttttcatgcttttggtagcagag